SEQUENCE LISTING (168 sequences)

SEQ ID NO: 1 - protein, 23 aa, INFLUENZA VIRUS
SLLTEVETPIRNEWGCRCNDSSD

SEQ ID NO: 2 - protein, 23 aa, INFLUENZA VIRUS
SLLTEVETPIRNEWGSRSNDSSD

SEQ ID NO: 3 - protein, 23 aa, INFLUENZA VIRUS
SLLTEVETPIRNEWGCRSNDSSD

SEQ ID NO: 4 - protein, 23 aa, INFLUENZA VIRUS
SLLTEVETPIRNEWGSRCNDSSD

SEQ ID NO: 5 - protein, 57 aa, INFLUENZA VIRUS
SLLTEVETPIRNEWGCRCNDSSDPLMKQIEDKLEEILSKLYHIENELARIKKLLGER

SEQ ID NO: 6 - protein, 44 aa, INFLUENZA VIRUS
MSLLTEVETPIRNEWGCRCNDSSDPLVVAASIIGILHLILWILD

SEQ ID NO: 7 - protein, 30 aa, INFLUENZA VIRUS
SLLTEVETPIRNEWGCRCNDSSDPLVVAAS

SEQ ID NO: 8 - protein, 16 aa, INFLUENZA VIRUS
SLLTEVETPIRNEWGC

SEQ ID NO: 9 - protein, 15 aa, INFLUENZA VIRUS
SSLTEVETPIRNEWG

SEQ ID NO: 10 - protein, 15 aa, INFLUENZA VIRUS
SLLTEVETPIRNEWG

SEQ ID NO: 11 - protein, 15 aa, INFLUENZA VIRUS
SLLTEVETPIRNEWG

SEQ ID NO: 12 - protein, 15 aa, INFLUENZA VIRUS
SLLTEVETPIRNEWG

SEQ ID NO: 13 - protein, 15 aa, INFLUENZA VIRUS
SLLTEVETPIRNEWG

SEQ ID NO: 14 - protein, 15 aa, INFLUENZA VIRUS
SLLTEVETPIRNEWG

SEQ ID NO: 15 - protein, 21 aa, INFLUENZA VIRUS
CGPEKQTRGLFGAIAGFIENG

SEQ ID NO: 16 - protein, 29 aa, INFLUENZA VIRUS
RVIEKTNEKFHQIEKEFSEVEGRIQDLEK

SEQ ID NO: 17 - protein, 20 aa, INFLUENZA VIRUS
KIDLWSYNAELLVALENQHT

SEQ ID NO: 18 - protein, 12 aa, INFLUENZA VIRUS
SLLTEVETPIRN

SEQ ID NO: 19 - protein, 14 aa, INFLUENZA VIRUS
SLLTEVETPIRNEW

SEQ ID NO: 20 - protein, 13 aa, INFLUENZA VIRUS
SLLTEVETPIRNE

SEQ ID NO: 21 - protein, 23 aa, INFLUENZA VIRUS
SLLTEVETPARNEWGSRSNDSSD

SEQ ID NO: 22 - protein, 23 aa, INFLUENZA VIRUS
SLLTEVETPIANEWGSRSNDSSD

SEQ ID NO: 23 - protein, 23 aa, INFLUENZA VIRUS
SLLTEVETPIRNEWGSRSNDSSD

SEQ ID NO: 24 - protein, 12 aa, INFLUENZA VIRUS
LTEVETPIRNEW

SEQ ID NO: 25 - protein, 13 aa, INFLUENZA VIRUS
LTEVETAPIRNEW

SEQ ID NO: 26 - protein, 23 aa, INFLUENZA VIRUS
SLLTEVATPIRNEWGSRSNDSSD

SEQ ID NO: 27 - protein, 23 aa, INFLUENZA VIRUS
SLLTEAETPIRNEWGSRSNDSSD

SEQ ID NO: 28 - protein, 23 aa, INFLUENZA VIRUS
ALLTEVETPIRNEWGSRSNDSSD

SEQ ID NO: 29 - protein, 23 aa, INFLUENZA VIRUS
SLATEVETPIRNEWGSRSNDSSD

SEQ ID NO: 30 - protein, 23 aa, INFLUENZA VIRUS
SALTEVETPIRNEWGSRSNDSSD

SEQ ID NO: 31 - protein, 23 aa, INFLUENZA VIRUS
SLLTEVETPIRNEWASRSNDSSD

SEQ ID NO: 32 - protein, 23 aa, INFLUENZA VIRUS
SLLTEVETPIRNEWGSRSNDSSA

SEQ ID NO: 33 - protein, 23 aa, INFLUENZA VIRUS
SLLTEVETPIRNEWGSRSNDSAD

SEQ ID NO: 34 - protein, 23 aa, INFLUENZA VIRUS
SLLTEVETPIRNEWGSRSNDASD

SEQ ID NO: 35 - protein, 23 aa, INFLUENZA VIRUS
SLLTEVETPIRNEWGSRSNASSD

SEQ ID NO: 36 - protein, 23 aa, INFLUENZA VIRUS
SLLTEVETPIRNEWGSRSADSSD

SEQ ID NO: 37 - protein, 23 aa, INFLUENZA VIRUS
SLLTEVETPIRNEWGSRANDSSD

SEQ ID NO: 38 - protein, 23 aa, INFLUENZA VIRUS
SLLTEVETPIRNEWGSRSNDSSD

SEQ ID NO: 39 - protein, 23 aa, INFLUENZA VIRUS
SLLTEVETPIRNEWGSRSNDSSD

SEQ ID NO: 40 - protein, 23 aa, INFLUENZA VIRUS
SLLTEVETPIRNEWGSRSNDSSD

SEQ ID NO: 41 - protein, 12 aa, INFLUENZA VIRUS
LTEVETPIRNEW

SEQ ID NO: 42 - protein, 23 aa, INFLUENZA VIRUS
SLLTEVETAIRNEWGSRSNDSSD

SEQ ID NO: 43 - protein, 23 aa, INFLUENZA VIRUS
SLLTEVETAIRNEWGSRSNDSSD

SEQ ID NO: 44 - protein, 23 aa, INFLUENZA VIRUS
SLLTEVEAPIRNEWGSRSNDSSD

SEQ ID NO: 45 - protein, 23 aa, INFLUENZA VIRUS
SLLTAVETPIRNEWGSRSNDSSD

SEQ ID NO: 46 - protein, 23 aa, INFLUENZA VIRUS
SLLAEVETPIRNEWGSRSNDSSD

SEQ ID NO: 47 - protein, 23 aa, INFLUENZA VIRUS
SLLTEVETPIRNEWGSASNDSSD

SEQ ID NO: 48 - protein, 23 aa, INFLUENZA VIRUS
SLLTEVETPIRNEWGARSNDSSD

SEQ ID NO: 49 - protein, 21 aa, INFLUENZA VIRUS
SLLTEVPIRNEWGSRSNDSSD

SEQ ID NO: 50 - protein, 23 aa, INFLUENZA VIRUS
SLLTEVETPARNEWGSRSNDSSD

SEQ ID NO: 51 - protein, 23 aa, INFLUENZA VIRUS
SLLTEVETPIRNEAGSRSNDSSD

SEQ ID NO: 52 - protein, 23 aa, INFLUENZA VIRUS
SLLTEVETPIRNAWGSRSNDSSD

SEQ ID NO: 53 - protein, 23 aa, INFLUENZA VIRUS
SLLTEVETPIRAEWGSRSNDSSD

SEQ ID NO: 54 - protein, 23 aa, INFLUENZA VIRUS
SLLTEVETPIANEWGSRSNDSSD

SEQ ID NO: 55 - protein, 25 aa, INFLUENZA VIRUS
SLLTEVETPIRNEWGDRSNDSSDAC

SEQ ID NO: 56 - protein, 25 aa, INFLUENZA VIRUS
SLLTEVETPIRNEWGDRSNDSSDAC

SEQ ID NO: 57 - protein, 25 aa, INFLUENZA VIRUS
SLLTEVETPIRNEWGDRSNDSSDAC

SEQ ID NO: 58 - protein, 25 aa, INFLUENZA VIRUS
SLLTEVETPIRNEWGDRSNDSSDAC

SEQ ID NO: 59 - protein, 21 aa, INFLUENZA VIRUS
CKIDLWSYNAELLVALENQHT

SEQ ID NO: 60 - protein, 22 aa, INFLUENZA VIRUS
EGPAKLLKERGFFGAIAGFLEE

SEQ ID NO: 61 - protein, 27 aa, INFLUENZA VIRUS
CEGLRNIPSIQSRGLFGAIAGFIEGGE

SEQ ID NO: 62 - protein, 27 aa, INFLUENZA VIRUS
CEGMRNVPEKQTRGLFGAIAGFIENGE

SEQ ID NO: 63 - protein, 27 aa, INFLUENZA VIRUS
GLFGAIAGFIENGWEGMIDGGCGKKKK

SEQ ID NO: 64 - protein, 15 aa, INFLUENZA VIRUS
CIEKTNEKFHQIEKE

SEQ ID NO: 65 - protein, 36 aa, INFLUENZA VIRUS
CRVIEKTNEKFHQIEKEFSEVEGRIQDLEKYVEDTK

SEQ ID NO: 66 - protein, 24 aa, INFLUENZA VIRUS
IEKEFSEVEGRIQDLEKYVEDTKC

SEQ ID NO: 67 - protein, 69 aa, INFLUENZA VIRUS
DQINGKLNRVIEKTNEKFHQIEKEFSEVEGRIQDLEKYVEDTKIDLWSYNAELLVALENQHTIDLKGGC

SEQ ID NO: 68 - protein, 72 aa, INFLUENZA VIRUS
CGGDQINGKLNRVIEKTNEKFHQIEKEFSEVEGRIQDLEKYVEDTKIDLWSYNAELLVALENQHTIDLKGGC

SEQ ID NO: 69 - protein, 22 aa, INFLUENZA VIRUS
CRTRKQLRENAEDMGNGAFKIY

SEQ ID NO: 70 - protein, 35 aa, INFLUENZA VIRUS
CGGRIQDLEKYVEDTKIDLWSYNAELLVALENQHT

SEQ ID NO: 71 - protein, 21 aa, INFLUENZA VIRUS
CGWYGFRHQNSEGTGQAADLK

SEQ ID NO: 72 - protein, 15 aa, INFLUENZA VIRUS
GLFGAIAGFIENGCE

SEQ ID NO: 73 - protein, 15 aa, INFLUENZA VIRUS
GLFGAIAGFIENGCE

SEQ ID NO: 74 - protein, 15 aa, INFLUENZA VIRUS
GLFGAIAGFIENGCE

SEQ ID NO: 75 - protein, 16 aa, INFLUENZA VIRUS
CGGLFGAIAGFIENGE

SEQ ID NO: 76 - protein, 22 aa, INFLUENZA VIRUS
GLFGAIAGFIENGWEGMVDGCE

SEQ ID NO: 77 - protein, 22 aa, INFLUENZA VIRUS
GLFGAIAGFIENGWEGMVDGCE

SEQ ID NO: 78 - protein, 19 aa, INFLUENZA VIRUS
CGQTRGLFGAIAGFIENGE

SEQ ID NO: 79 - protein, 22 aa, INFLUENZA VIRUS
GIFGAIAGFIENGWEGMVDGCE

SEQ ID NO: 80 - protein, 22 aa, INFLUENZA VIRUS
GLFGAIAGFIEGGWTGMIDGCE

SEQ ID NO: 81 - protein, 24 aa, INFLUENZA VIRUS
GLFGAIAGFIENGWEGMVDGKKCE

SEQ ID NO: 82 - protein, 24 aa, INFLUENZA VIRUS
GLFGAIAGFIEGGWTGMIDGKKCE

SEQ ID NO: 83 - protein, 21 aa, INFLUENZA VIRUS
CGPEKQTRGLFGAIAGFIENG

SEQ ID NO: 84 - protein, 26 aa, INFLUENZA VIRUS
PEKQTRGLFGAIAGFIENGGCGKKKK

SEQ ID NO: 85 - protein, 17 aa, INFLUENZA VIRUS
PEKQTRGLFGAIAGFIC

SEQ ID NO: 86 - protein, 13 aa, INFLUENZA VIRUS
CGPEKQTRGLFGA

SEQ ID NO: 87 - protein, 20 aa, INFLUENZA VIRUS
PEKQTRGLFGAIAGFIENGC

SEQ ID NO: 88 - protein, 25 aa, INFLUENZA VIRUS
GMRNVPEKQTRGLFGAIAGFIENGC

SEQ ID NO: 89 - protein, 12 aa, INFLUENZA VIRUS
CGPEKQTRGLFG

SEQ ID NO: 90 - protein, 11 aa, INFLUENZA VIRUS
CGPEKQTRGLF

SEQ ID NO: 91 - protein, 10 aa, INFLUENZA VIRUS
CGPEKQTRGL

SEQ ID NO: 92 - protein, 9 aa, INFLUENZA VIRUS
CGPEKQTRG

SEQ ID NO: 93 - protein, 25 aa, INFLUENZA VIRUS
CGMRNVPEKQTRGLFGAIAGFIENG

SEQ ID NO: 94 - protein, 25 aa, INFLUENZA VIRUS
CGMRNVPEKQTRGLFGAIAGFIENG

SEQ ID NO: 95 - protein, 12 aa, INFLUENZA VIRUS
CGPEKQTRGLFG

SEQ ID NO: 96 - protein, 11 aa, INFLUENZA VIRUS
CGPEKQTRGLF

SEQ ID NO: 97 - protein, 10 aa, INFLUENZA VIRUS
CGPEKQTRGL

SEQ ID NO: 98 - protein, 9 aa, INFLUENZA VIRUS
CGPEKQTRG

SEQ ID NO: 99 - protein, 25 aa, INFLUENZA VIRUS
CGMRNVPEKQTRGLFGAIAGFIENG

SEQ ID NO: 100 - protein, 23 aa, INFLUENZA VIRUS
CGNVPEKQTRGLFGAIAGFIENG

SEQ ID NO: 101 - protein, 21 aa, INFLUENZA VIRUS
CGPEKQTRGLFGAIAGFIENG

SEQ ID NO: 102 - protein, 22 aa, INFLUENZA VIRUS
CGPEKQTRGLFGAIAGFIENGE

SEQ ID NO: 103 - protein, 22 aa, INFLUENZA VIRUS
CGPEKQTRGLFGAIAGFIENGE

SEQ ID NO: 104 - protein, 21 aa, INFLUENZA VIRUS
EPEKQTRGLFGAIAGFIENGC

SEQ ID NO: 105 - protein, 20 aa, INFLUENZA VIRUS
GPEKQTRGLFGAIAGFIENG

SEQ ID NO: 106 - protein, 20 aa, INFLUENZA VIRUS
GPSIQSRGLFGAIAGFIEGG

SEQ ID NO: 107 - protein, 21 aa, INFLUENZA VIRUS
CGPSIQSRGLFGAIAGFIEGG

SEQ ID NO: 108 - protein, 21 aa, INFLUENZA VIRUS
CGPEKQTRGIFGAIAGFIENG

SEQ ID NO: 109 - protein, 19 aa, INFLUENZA VIRUS
GPEKQTRGIFGAIAGFIEE

SEQ ID NO: 110 - protein, 20 aa, INFLUENZA VIRUS
EGPEKQTRGIFGAIAGFIEE

SEQ ID NO: 111 - protein, 19 aa, INFLUENZA VIRUS
GPSIQSRGLFGAIAGFIEE

SEQ ID NO: 112 - protein, 20 aa, INFLUENZA VIRUS
EGPSIQSRGLFGAIAGFIEE

SEQ ID NO: 113 - protein, 21 aa, INFLUENZA VIRUS
CEGPEKQTRGIFGAIAGFIEE

SEQ ID NO: 114 - protein, 20 aa, INFLUENZA VIRUS
CGPSIQSRGLFGAIAGFIEE

SEQ ID NO: 115 - protein, 21 aa, INFLUENZA VIRUS
CEGPSIQSRGLFGAIAGFIEE

SEQ ID NO: 116 - protein, 29 aa, INFLUENZA VIRUS
CEGPEKQTRGLFGAIAGFIENGWEGMIDE

SEQ ID NO: 117 - protein, 19 aa, INFLUENZA VIRUS
GPSIQSRGLFGAIAGFIEE

SEQ ID NO: 118 - protein, 21 aa, INFLUENZA VIRUS
CEGPEKQTRGLFGAIAGFIEE

SEQ ID NO: 119 - protein, 23 aa, INFLUENZA VIRUS
CRGLFGAIAGFIEGGWTGMIDGE

SEQ ID NO: 120 - protein, 34 aa, INFLUENZA VIRUS
CEGLRNIPSIQSRGLFGAIAGFIEGGWTGMIDGE

SEQ ID NO: 121 - protein, 25 aa, INFLUENZA VIRUS
CRGLFGAIAGFIEGGWTGMIDGKKE

SEQ ID NO: 122 - protein, 36 aa, INFLUENZA VIRUS
CEGLRNIPSIQSRGLFGAIAGFIEGGWTGMIDGKKE

SEQ ID NO: 123 - protein, 16 aa, INFLUENZA VIRUS
CEGLRNIPSIQSRGLE

SEQ ID NO: 124 - protein, 26 aa, INFLUENZA VIRUS
EGMRNVPEKQTRGLFGAIAGFIENGE

SEQ ID NO: 125 - protein, 26 aa, INFLUENZA VIRUS
EGLRNIPSIQSRGLFGAIAGFIEGGE

SEQ ID NO: 126 - protein, 21 aa, INFLUENZA VIRUS
GPAKLLKERGFFGAIAGFLEE

SEQ ID NO: 127 - protein, 22 aa, INFLUENZA VIRUS
CGPAKLLKERGFFGAIAGFLEE

SEQ ID NO: 128 - protein, 23 aa, INFLUENZA VIRUS
CEGPAKLLKERGFFGAIAGFLEE

SEQ ID NO: 129 - protein, 21 aa, INFLUENZA VIRUS
EGAKLLKERGFFGAIAGFLEE

SEQ ID NO: 130 - protein, 22 aa, INFLUENZA VIRUS
EGPAKLLKERGFFGAIAGFLEE

SEQ ID NO: 131 - protein, 22 aa, INFLUENZA VIRUS
EGPAKLLKERGFFGAIAGFLEE

SEQ ID NO: 132 - protein, 22 aa, INFLUENZA VIRUS
EGPAKLLKERGFFGAIAGFLEE

SEQ ID NO: 133 - protein, 22 aa, INFLUENZA VIRUS
EGPAKLLKERGFFGAIAGFLEE

SEQ ID NO: 134 - protein, 20 aa, INFLUENZA VIRUS
GPAKLLKERGFFGAIAGFLE

SEQ ID NO: 135 - protein, 18 aa, INFLUENZA VIRUS
AKLLKERGFFGAIAGFLE

SEQ ID NO: 136 - protein, 17 aa, INFLUENZA VIRUS
KLLKERGFFGAIAGFLE

SEQ ID NO: 137 - protein, 16 aa, INFLUENZA VIRUS
LLKERGFFGAIAGFLE

SEQ ID NO: 138 - protein, 15 aa, INFLUENZA VIRUS
LKERGFFGAIAGFLE

SEQ ID NO: 139 - protein, 14 aa, INFLUENZA VIRUS
KERGFFGAIAGFLE

SEQ ID NO: 140 - protein, 13 aa, INFLUENZA VIRUS
ERGFFGAIAGFLE

SEQ ID NO: 141 - protein, 12 aa, INFLUENZA VIRUS
RGFFGAIAGFLE

SEQ ID NO: 142 - protein, 11 aa, INFLUENZA VIRUS
GFFGAIAGFLE

SEQ ID NO: 143 - protein, 19 aa, INFLUENZA VIRUS
GPAKLLKERGFFGAIAGFL

SEQ ID NO: 144 - protein, 18 aa, INFLUENZA VIRUS
GPAKLLKERGFFGAIAGF

SEQ ID NO: 145 - protein, 17 aa, INFLUENZA VIRUS
GPAKLLKERGFFGAIAG

SEQ ID NO: 146 - protein, 16 aa, INFLUENZA VIRUS
GPAKLLKERGFFGAIA

SEQ ID NO: 147 - protein, 15 aa, INFLUENZA VIRUS
GPAKLLKERGFFGAI

SEQ ID NO: 148 - protein, 14 aa, INFLUENZA VIRUS
GPAKLLKERGFFGA

SEQ ID NO: 149 - protein, 13 aa, INFLUENZA VIRUS
GPAKLLKERGFFG

SEQ ID NO: 150 - protein, 12 aa, INFLUENZA VIRUS
GPAKLLKERGFF

SEQ ID NO: 151 - protein, 11 aa, INFLUENZA VIRUS
GPAKLLKERGF

SEQ ID NO: 152 - protein, 10 aa, INFLUENZA VIRUS
GPAKLLKERG

SEQ ID NO: 153 - protein, 9 aa, INFLUENZA VIRUS
GPAKLLKER

SEQ ID NO: 154 - protein, 22 aa, INFLUENZA VIRUS
AGPAKLLKERGFFGAIAGFLEE

SEQ ID NO: 155 - protein, 22 aa, INFLUENZA VIRUS
EGAAKLLKERGFFGAIAGFLEE

SEQ ID NO: 156 - protein, 22 aa, INFLUENZA VIRUS
EGPAALLKERGFFGAIAGFLEE

SEQ ID NO: 157 - protein, 22 aa, INFLUENZA VIRUS
EGPAKALKERGFFGAIAGFLEE

SEQ ID NO: 158 - protein, 22 aa, INFLUENZA VIRUS
EGPAKLAKERGFFGAIAGFLEE

SEQ ID NO: 159 - protein, 22 aa, INFLUENZA VIRUS
EGPAKLLAERGFFGAIAGFLEE

SEQ ID NO: 160 - protein, 22 aa, INFLUENZA VIRUS
EGPAKLLKARGFFGAIAGFLEE

SEQ ID NO: 161 - protein, 22 aa, INFLUENZA VIRUS
EGPAKLLKEAGFFGAIAGFLEE

SEQ ID NO: 162 - protein, 22 aa, INFLUENZA VIRUS
EGPAKLLKERGAFGAIAGFLEE

SEQ ID NO: 163 - protein, 22 aa, INFLUENZA VIRUS
EGPAKLLKERGFAGAIAGFLEE

SEQ ID NO: 164 - protein, 22 aa, INFLUENZA VIRUS
EGPAKLLKERGFFGAAAGFLEE

SEQ ID NO: 165 - protein, 22 aa, INFLUENZA VIRUS
EGPAKLLKERGFFGAIAGALEE

SEQ ID NO: 166 - protein, 22 aa, INFLUENZA VIRUS
EGPAKLLKERGFFGAIAGFAEE

SEQ ID NO: 167 - protein, 22 aa, INFLUENZA VIRUS
EGPAKLLKERGFFGAIAGFLAE

SEQ ID NO: 168 - protein, 22 aa, INFLUENZA VIRUS
EGPAKLLKERGFFGAIAGFLEA

(19) United States
(12) Patent Application Publication
(10) Pub. No.: US 2004/0223976 A1
(43) Pub. Date: Nov. 11, 2004

(54) Influenza virus vaccine

(76) Inventors: Elisabetta Bianchi, Roma (IT); Victor M. Garsky, Blue Bell, PA (US); Paolo Ingallinella, Roma (IT); Roxana Ionescu, Collegeville, PA (US); Xiaoping Liang, Eagleville, PA (US); Antonello Pessi, Roma (IT); Craig T. Przysiecki, Lansdale, PA (US); Li Shi, Eagleville, PA (US); John W. Shiver, Chalfont, PA (US)

Correspondence Address: MERCK AND CO INC, P O BOX 2000, RAHWAY, NJ, 070650907

(21) Appl. No.: 10/794,646

(22) Filed: Mar. 5, 2004

Related U.S. Application Data
(60) Provisional application No. 60/452,749, filed on Mar. 7, 2003.
(60) Provisional application No. 60/530,690, filed on Dec. 18, 2003.

(51) Int. Cl. A61K 39/12; A61K 39/21; C07K 14/11

(52) U.S. Cl. 424/186.1; 530/350

(57) ABSTRACT

The present invention provides vaccines against disease caused by infection with influenza virus, and methods of vaccination. The vaccines comprise peptides derived from the M2 and/or HA proteins of influenza virus conjugated to a carrier protein.

### CROSS-REFERENCE TO RELATED APPLICATIONS

The present application claims the benefit of U.S. Provisional Application Nos. 60/452,749 and 60/530,690 filed Mar. 7, 2003 and Dec. 18, 2003, respectively, hereby incorporated by reference herein.

### FIELD OF THE INVENTION

This invention relates to the field of vaccines, vaccination and therapies for the prevention and treatment of maladies implicating influenza virus.

### BACKGROUND OF THE INVENTION

Influenza virus, an enveloped, segmented negative strand RNA virus occurs in two major types, influenza A and influenza B. The virus is the infectious agent responsible for causing flu in humans. Influenza A viruses are further divided into subtypes, based on the antigenic difference of the two viral transmembrane proteins, hemagglutinin (HA) and neuraminidase (NA). To date, 3 subtypes of influenza A have been identified in humans, H1N1, H₂N₂ and H₃N₂ (Hilleman, Vaccine 20, 3068-3087, 2002). The influenza B virus, which circulates almost exclusively in humans, is characterized by a lower rate of antigenic change. Recent isolates of influenza B virus are classified into two major phylogenetic trees, the influenza B/Victoria/2/87 subclass and the influenza B/Yamagata/16/88 subclass. These two lineages are antigenically and genetically distinct, such that little or no post-infection cross-neutralizing antibody response is observed in ferrets (Rota et al., J Gen Virol 73 (Pt 10), 2737-42 (1992).

The segmented nature of the influenza virus genome allows for reassortment of segments during virus replication in superinfected cells. The reassortment of segments, combined with genetic mutation and drift, gives rise to myriad divergent strains of influenza within each serotype group over time. The new strains exhibit antigenic variation in their hemagglutinin and/or neuraminidase proteins.

The predominant current practice for the prevention of flu is annual vaccination. Most commonly, whole virus vaccines are used. They must contain an influenza A H1N1 strain, an influenza A H3N₂ strain, and an influenza B strain. However, due to constant antigenic variation of influenza transmembrane proteins, a single vaccine against those proteins is not appropriate for use from year to year. Therefore, the myriad strains in the at-large population of influenza viruses are characterized, tracked and forecast. Based on the prevalence and forecast for individual strains of virus during a given year, a vaccine is designed to stimulate a protective immune response against the predominant and expected viral strains.

Compared to the use of vaccines for which a single course of vaccination provides protection for numerous years or lifetime protection, the process of annual vaccination is inconvenient for patients and medical practitioners, inconsistently applied across the patient population, does not provide cross-protection against other influenza virus strains within a given serotype group and results in lives lost to influenza infection. Therefore, a vaccine against influenza that could be given in a single course of inoculation, could provide cross-protection against new strains in a highly divergent population of viruses, and could provide such protection for a number of years or for the lifetime of a vaccinee would be of great benefit.

A vaccine based on a stable influenza antigen common to all strains of a given influenza type could provide such benefits. Recently, the M2 protein of influenza type A has been investigated as antigenic protein that could form the basis of such a vaccine (Slepushkin et al., 1995 Vaccine 13:1399-1402). The M2 protein is a structurally conserved viral surface protein. M2 is a relatively minor component of the influenza virion (Zebedee and Lamb, 1988 J. Virol. 62:2762-2772), but is abundantly expressed in infected cells during virus infection (Lamb et al., 1985 Cell 40:627-633). In infected cells, M2 appears in the cellular membrane and provides proton flux for viral replication (Helenius, 1992 Cell 69:577-578).

The replication of influenza A was stated to be inhibited by antibodies against M2 in both in vivo and in vitro models of infection (Zebedee and Lamb, 1988 J. Virol. 62:2762-2772; Hughey et al., 1995 Virology 212:411-421). Slepushkin et al., 1995 Vaccine 13:1399-1402, described an experiment wherein mice vaccinated with full length M2 were protected against a lethal challenge of heterologous influenza A and exhibited enhanced clearance of virus from infected lung tissue.

More recently, modified M2 proteins in which the hydrophobic transmembrane domain had been removed were reported to be useful for making a vaccine (U.S. Pat. No. 6,169,175). In another vein, Neirynck et al., 1999 Nature Med. 5: 1157-1163, described the use of a fusion of the extracellular domain of M2 to the N-terminus of Hepatitis B core antigen. When the Hepatitis core antigen was incorporated into viral-like particles, the M2 epitope was said to be presented as part of the exposed N-terminus of the Hepatitis B core antigen. The authors stated that in their system, the N-terminal fusion to Hepatitis B core antigen presented the M2 epitope in a way that mimicked the wild-type structure of the M2 protein in viral particles and infected cells.

However, this approach cannot be extended to influenza B virus, for lack of a vaccine target equivalent to M2. The most likely candidate protein for an M2-equivalent function in influenza B virus, BM₂, has an extremely short extracellular domain of only 5-7 amino acids (Mould et al., Developmental Cell 5, 175-184, 2003). An alternative candidate protein, NB, was recently shown to be dispensable for viral replication in vitro (Hatta et al., J. Virol. 77, 6050-6054, 2003).

An alternative approach to the development of a universal influenza B vaccine is based on the maturational cleavage site of the HA precursor, called HA₀. A vaccine targeting conserved epitopes of HA, and in particular conserved epitopes of HA₀, would be applicable to both influenza type A and influenza type B.

The envelope glycoprotein HA mediates both the initial attachment of the virus and its subsequent internalization (Skehel et al., Annual Review of Biochemistry 69, 531-69, 2000). HA is composed of two subunits, HA₁ and HA₂, that are cleaved from their precursor HA₀ (Skehel et al., Proc Natl Acad Sci USA 72, 93-7 (1975; Chen et al., Cell 95, 409-17, 1998). HA₀ maturation is a cell-associated process, mediated by proteases secreted by the cells in which the virus is replicating (Zhirnov, Biochemistry (Mosc) 68, 1020-6 (2003). Many secreted enzymes have been associated with HA₀ cleavage, including plasmin, kallikrein, urokinase, thrombin, blood clotting factor Xa, acrosin, tryptase Clara, tryptase TC30, mini-plasmin, proteases from human respiratory lavage, and bacterial proteases from Staphylococcus aureus and Pseudomonas aeruginosa. Cleavage of HA₀ into HA₁-HA₂ activates virus infectivity (Klenk et al., Virology 68, 426-39, 1975; Lazarowiz & Choppin, Virology 68, 440-54 (1975) and is crucial to pathogenicity in human and avian hosts (Klenk & Garten, Trends Microbiol 2, 3943 1994; Steinhauer, Virology 258, 1-20, 1999).

The major characteristics of HA that determines its sensitivity to host proteases is the composition of the proteolytic site of the HA₀ precursor, whose structure was recently solved for the influenza A virus by X-ray crystallography (Chen et al., Cell 95, 409-17, 1998). HA₀ is almost identical to the mature processed HA₁-HA₂ protein, differing primarily in the 18 residues surrounding the cleavage site. In the precursor, these residues are folded as an extended, uncleaved loop. The amino acid sequence of the intersubunit cleavage site is highly conserved within each influenza subtype, and within the two lineages of influenza B virus. The HA₂ side, which corresponds to the fusion peptide, is also conserved across influenza A subtypes, being almost identical for H3 and H1, and for influenza B as well.

Throughout the specification, the term HA₀ peptides is used to indicate any peptide derived from the primary sequence of HA₀. This includes the cleavage site sequence, which is unique to HA₀, but also any sequence shared by the HA₀ precursor and the mature HA. Mature HA is, in turn, composed of the two covalently linked subunits HA₁ and HA₂. For this reason, HA₀ peptides different from the cleavage site sequence are referred to, alternatively, as HA peptides, or HA₂ peptides. Each of these terms refers to a type of peptide within the class herein referred to a HA₀ peptides.

The feasibility of this approach was first explored by Nagy et al., who showed that mice vaccinated with a synthetic peptide corresponding to sequence 317-341 of HA₀ (subtype H1) were partially protected from lethal viral challenge (Nagy et al., Scand J Immunol 40, 281-91, 1994). Further validation of the HA₀ to HA₁-HA₂ conversion as a vaccine target comes from the effect of protease inhibitors on viral replication. In influenza viruses with monobasic cleavage sites, serine protease inhibitors are able to reduce HA₀ cleavage and virus activation in cultured cells, in human respiratory epithelium and in the lungs of infected mice (Zhirnov et al., J Gen Virol 63, 469-74, 1982; Zhirnov et al., J Gen Virol 65, 191-6, 1984; Zhirnov et al., J Virol 76, 8682-9, 2002).

### SUMMARY OF THE INVENTION

An aspect of the present invention is a protein-peptide conjugate, or a pharmaceutically acceptable salt thereof, in which a multitude of peptides, each of which comprises an extracellular epitope of the M2 protein of type A influenza virus, are conjugated to the surface of a carrier protein.

Another aspect of the present invention is a protein-peptide conjugate, or a pharmaceutically acceptable salt thereof, in which a multitude of peptides, each of which comprises an epitope of the HA₀ protein of type A influenza virus, are conjugated to the surface of a carrier protein.

Another aspect of the present invention is a protein-peptide conjugate, or a pharmaceutically acceptable salt thereof, in which a multitude of peptides, each of which comprises an epitope of the HA₀ protein of type B influenza virus, are conjugated to the surface of a carrier protein.

In particular embodiments, the peptides are conjugated to the protein by covalently joining peptides to reactive sites on the surface of the protein. The resulting structure is a conjugate. A reactive site on the surface of the protein is a site that is chemically active or that can be activated and is sterically accessible for covalent joining with a peptide. A preferred reactive site is the epsilon nitrogen of the amino acid lysine. Covalently joined refers to the presence of a covalent linkage that is stable to hydrolysis under physiological conditions. Preferably, the covalent linkage is stable to other reactions that may occur under physiological conditions including adduct formation, oxidation, and reduction. The covalent joining of peptide to protein is achieved by “means for joining”. Such means cover the corresponding structure, material, or acts described herein and equivalents thereof.

In a particular embodiments of this aspect of the invention, the carrier protein is an antigenic protein useful in the art of vaccination. In a particular embodiment of the invention, the antigenic protein is the outer membrane protein complex (OMPC) of Neiserria meningitidis. In other embodiments, the carrier protein can be tetanus toxoid, diphtheria toxoid, Hepatitis B Surface Antigen (HBsAg), Hepatitis B core antigen (HBcAg), keyhole limpet hemocyanin, a Rotavirus capsid protein, or the LI protein of a bovine or human Papilloma Virus Virus Like Particle (VLP), for example a VLP of HPV type 6, 11 or 16.

In further embodiments of this aspect of the invention, the peptides are conjugated to the carrier protein via their N-terminus or their C-terminus.

In further embodiments, the peptide is conjugated to the carrier protein via a linker moiety. In particular embodiments, the linker is a monogeneric or bigeneric spacer.

In further embodiments, the carrier protein is the outer membrane protein complex (OMPC) of Neiserria meningitidis and the conjugate has from about 100 to about 6000 peptides conjugated to the surface of each OMPC.

In further embodiments, amino acids naturally occurring in the sequence of the peptides are replaced by other amino acids. In particular embodiments, cysteine residues are replaced by serine residues.

In further embodiments, the sequence of the peptide is modified to alter the isoelectric point of the peptide.

Another aspect of the invention is a vaccine having the conjugates, an adjuvant and a physiologically acceptable carrier. In particular embodiments the adjuvant is an aluminum based adjuvant. In particular embodiments, the vaccine further comprises a cationic adjuvant, e.g., the QS21 adjuvant.

Another aspect of this invention is a vaccine having a M2 conjugate and a conjugate of an HA₀ peptide from influenza type B, an adjuvant and a physiologically acceptable carrier.

Another aspect of this invention is a vaccine having a M2 conjugate and a conjugate of an HA₀ peptide from influenza type A and a conjugate of an HA₀ peptide from influenza type B, an adjuvant and a physiologically acceptable carrier.

Another aspect of the invention is a method of vaccination of a patient against disease caused by infection with type A influenza virus with a vaccine comprising a peptide-protein conjugate, or pharmaceutically acceptable salt thereof, in which a multitude of peptide, each comprising an extracellular epitope of the M2 protein of type A influenza virus, are conjugated to the surface of a carrier protein. In preferred embodiments, an effective amount of a vaccine of this invention is administered to a patient.

Another aspect of the invention is a method of vaccination of a patient against disease caused by infection with type A influenza virus with a vaccine of this invention comprising a protein-peptide conjugate, or a pharmaceutically acceptable salt thereof, in which a multitude of peptides, each of which comprises an epitope of the HA₀ protein of type A influenza virus, are conjugated to the surface of a carrier protein. In preferred embodiments, an effective amount of a vaccine of this invention is administered to a patient.

Another aspect of the invention is a method of vaccination of a patient against disease caused by infection with type A or B influenza virus with a vaccine comprising a protein-peptide conjugate, or a pharmaceutically acceptable salt thereof, in which a multitude of peptides, each of which comprises an epitope of the HA₀ protein of type A or B influenza virus, are conjugated to the surface of a carrier protein. In preferred embodiments, an effective amount of a vaccine of this invention is administered to a patient.

Another aspect of this invention is a method of making a peptide-protein conjugate by covalently linking peptides having the sequence of an extracellular epitope of the M2 protein of influenza to reactive sites on the surface of a protein.

Another aspect of this invention is a method of making a vaccine by adjuvanting a conjugate of this invention and formulating the adjuvanted conjugate with a pharmaceutically acceptable carrier.

Another aspect of the present invention is a combination vaccine wherein one of the antigenic components comprises peptides having an extracellular epitope of the M2 protein of type A influenza virus conjugated to amino acids on the surface of a carrier protein. In particular embodiments, the combination vaccine comprises antigenic components selected from Haemophilus influenza, hepatitis viruses A, B, or C, human papilloma virus, measles, mumps, rubella, varicella, rotavirus, Streptococcus pneumonia and Staphylococcus aureus. Additionally, the vaccine of the present invention can be combined with other antigenic components of influenza virus type A and influenza virus type B including, in particular, epitopes derived from hemagglutinin and neuraminidase.

### BRIEF DESCRIPTION OF THE DRAWINGS

FIG. 1. Reactions of thiolated carrier (1) with bromoacetylated (2) or maleimidated (3) peptides and resulting thiolether linkages (Scheme I).

FIG. 2. Reaction of carrier intrinsic primary amines (1) with bromoacetylated (2) or maleimidated (3) peptides and resulting secondary amine linkages (Scheme II).

FIG. 3. Reaction of maleimidated carrier (1) with thiol containing peptide (2) and creation of thiolether link (Scheme III). For peptides containing multiple thiols, multiple links with carrier maleimide groups can occur with a single peptide. This can reduce the total amount of peptide loading to the carrier. If the multiple links occur on maleimides on separate proteins, cross-linking of carrier subunits through the peptide can occur.

FIG. 4. Reaction of alkylhalide carrier (1) with thiol containing peptide (2) and creation of thiolether link (Scheme IV). For peptides containing multiple thiols, multiple links with carrier alkylhalide (iodoacetyl shown or bromoacetyl) groups can occur with a single peptide. This can reduce the total amount of peptide loading on the carrier. If the multiple links occur on iodoacetyl groups on separate proteins, cross-linking of carrier subunits through the peptide can occur.

FIG. 5. Hydrolysis of cross-linked maleimidated influenza peptides and thiolated OMPC. The non-protein amino acid S-(1,2-dicarboxyethyl)-homocysteine can be quantitated to provide evidence for covalent linkage. 4-aminobutyric acid and 6-aminohexanoic acid can be quantitated to estimate total peptide present (Scheme V).

FIG. 6. Hydrolysis of coupled bromoacetylated influenza peptides and thiolated OMPC. The non-protein amino acid S-(carboxymethyl)-homocysteine can be quantitated to provide evidence for covalent linkage. 6-aminohexanoic acid can be quantitated to estimate total peptide present (Scheme VI).

FIG. 7. Hydrolysis of coupled cysteine containing influenza peptides and iodoacetylated OMPC. The non-protein amino acid S-carboxymethyl-cysteine can be quantitated to provide evidence for covalent linkage. 6-aminohexanoic acid can be quantitated to estimate total peptide present. 4-aminobenzoic acid can be quantitated to estimate the total amount of cross-linker associated with the OMPC (Scheme VII).

FIG. 8. Hydrolysis of coupled cysteine containing Flu M2 peptides and maleimidated OMPC. The non-protein amino acid S-(1,2-dicarboxyethyl)-cysteine can be quantitated to provide evidence for covalent linkage. 6-aminohexanoic acid can be quantitated to estimate total peptide present. Tranexamic acid can be quantitated to estimate the total amount of cross-linker associated with the OMPC (Scheme VIII).

FIG. 9. Induction of M2-specific antibody responses by M2 peptide conjugate vaccines in mice. Female Balb/c mice, 10 per group, were immunized intramuscularly with 0.01 μg, 0.1 μg or 1 μg of a designated conjugate (dose based on the peptide weight), and boosted once with the same dose three weeks later. Blood samples were collected at two weeks after first immunization (PD1) and three weeks after the boost immunization (PD2). M2-specific antibody titers were determined by Enzyme-linked immunosorbent assay (Elisa). The data represent group geometric means+/−standard errors (GMT+/−SE). CT M2 15mer ma-OMPC, M2 15-mer (SEQ ID NO:10) conjugated via C terminal cysteine to maleimide-activated OMPC; CT BrAc-M2 15mer OMPC, C-terminal bromoacetylated M2 15-mer (SEQ ID NO:13) conjugated to thiolated OMPC; NT BrAc-M2 15mer OMPC, N-terminal bromoacetylated 15mer M2 peptide (SEQ ID NO:11) conjugated to thiolated OMPC; CT BrAc-M2(SRS) OMPC, C-terminal Bromoacetylated M2 23-mer (SRS) (SEQ ID NO:39) conjugated to thiolated OMPC. GMT=Geometric Mean Titer.

FIG. 10. Protection by CT M2 15mer ma-OMPC and CT BrAc-M2 15mer OMPC against lethal flu challenge. Per FIG. 9 legend for animal immunization protocol. Animals were challenged intranasally with LD90 of flu A/HK/68 reassortant four weeks after the boost immunization. Percent of weight change was calculated as: group average weight at day of test/group average weight at day 0 post challenge×100%. Percentage of survival was calculated as: number of animals at day of test/number of animals at day 0 post challenge×100%.

FIG. 11. Protection by CT BrAc-M2 15mer OMPC and CT BrAc-M2(SRS) OMPC against lethal flu challenge. Per FIG. 9 and FIG. 10 legend.

FIG. 12. Protection by CT BrAc-M2 15mer OMPC and NT M2 15mer ma-OMPC against lethal flu challenge. Per FIG. 9 and FIG. 10 legend.

FIG. 13A Conjugation of maleimide derivatized influenza peptide to thiolated OMPC.

FIG. 13B Conjugation of bromoacetylated influenza peptide to thiolated OMPC.

FIG. 14 Peptides, SEQ ID NO:12 and SEQ ID NO:14 are examples of peptides that can be linked to a carrier protein as shown schematically in FIG. 13a. Peptides SEQ ID NO:11 and SEQ ID NO:13 are examples peptides that can be linked to a carrier protein as shown schematically in FIG. 13b. Peptide SEQ ID NO:39 is a truncated form of the SRS M2 sequence with a C-terminal cysteine which can be conjugated to a thiol reactive derivative of OMPC or other carrier protein. SEQ ID NO:2 represents the longer M2 counterpart.

FIG. 15. A schematic representation of multiple M2 peptides on a lysine scaffold. R=SEQ ID NO: 8.

FIG. 16. A schematic representation of multiple M2 peptides on a lysine scaffold. R=SEQ ID NO: 1.

FIG. 17. A schematic representation of multiple M2 peptides on a lysine scaffold. R=SEQ ID NO: 2.

FIG. 18. A schematic representation of multiple M2 peptides on a lysine scaffold. R=SEQ ID NO: 2.

FIG. 19. A schematic representation of multiple M2 peptides linked together as a dimer. DAP=L-2,3-diaminopropionic acid. The top dimer includes SEQ ID NOs: 55 & 56. The bottom dimer includes SEQ ID NOs: 57 & 58.

FIG. 20. A schematic representation of multiple M2 peptides on a lysine scaffold. R=SEQ ID NO: 2. Introduction of a Cys residue to the structure represented by FIG. 18 provides a MAP with a free thiol functionality as shown in FIGS. 17 and 20. Such MAPs may be used for conjugation to carrier proteins containing bromoacetyl, maleimide or other thiol reactive groups.

FIG. 21. A schematic representation of multiple M2 peptides on multiple lysine scaffolds wherein the scaffolds are linked together. R=SEQ ID NO: 2.

FIG. 22A. HA₀-specific antibody responses against an Influenza type B peptide-conjugate vaccine.

FIG. 22B. Survival curves after influenza B virus challenge in mice vaccinated with an Influenza type B peptide-conjugate vaccine.

FIG. 23. The effects of influenza type B vaccine component on in vivo viral replication was tested in a sublethal challenge model.

FIG. 24. Survival curves for mice immunized with an Influenza type A HA₂ peptide conjugate vaccine.

FIG. 25. Ribbon diagram of the L1 protein as determined by X-ray in a 12-capsomere VLP (Chen et al., “Structure of small virus-like-particles assembled from the L1 protein of human papillomavirus 16”, Mol. Cell., Vol. 5, pp. 557-567, 2000). The individual medium gray spheres represent the NZ atoms of 19 Lys chains that are on the exterior surface of the VLP. The dark gray cluster shows Phe 50 that is part of the epitope for both H16.V5 and H16.E70 antibodies. The light gray cluster represents the binding loop for H16.J4 antibody. The figure was generated using the program MolMol (Koradi, R., Billeter, M., and Wutrich, K. 1996. MOLMOL: a program for display and analysis of macromolecular structures. J. Mol. Graphics 14, 51-55)

FIGS. 26A & 26B. Particle size distribution for HPV VLP type 16 (solid line), activated/quenched HPV-VLP (dashed line) and conjugate M2-HPV VLP (solid line with circles) as determined by (27A) SEC-HPLC and (27B) Analytical Ultracentrifugation.

FIG. 27. Electron microscopy image of M2-HPV VLP.

FIG. 28. Temperature-induced aggregation monitored by OD at 350 nm for HPV VLP type 16 (solid line), activated/quenched HPV-VLP (dashed line) and conjugate M2-HPV VLP (solid line with circles).

FIGS. 29A & 29B. 29A: Geometric Mean Titer (GMT) of anti-M2 antibody induced by M2-HPV VLP in mice at T=2 and 6 weeks after immunizations at T=0 and T=4 weeks with vaccines containing M2-HPV VLP at different peptide doses. 29B: Rate of survival against lethal challenge for mice immunized with vaccines containing M2-HPV VLP at different peptide doses.

FIG. 30. Protection by immunization with M2-KLH conjugate vaccine against nasal and lung viral shedding in mice. Viral shedding profiles in upper and the lower respiratory tracts following sub-lethal viral challenge in mice. Data represent GMT+/−S.E. of eight mice at each data time point. The dash line is the assay detection threshold. GMT=Geometric Mean Titer.

FIG. 31. Induction of antibody responses in rhesus monkeys by M2-OMPC conjugate vaccine. Thirty rhesus monkeys were divided into 10 groups of three animals each. Each data point represents the average GMT of three animals per group. Mean/Alum stands for the GMT of all four groups of either OMPC immune or OMPC naive monkeys that received M2-OMPC formulated in Alum. GMT=Geometric Mean Titer.

### DETAILED DESCRIPTION OF THE INVENTION

The present invention provides an influenza vaccine in which a multitude of peptides comprising an extracellular epitope of the M2 protein of influenza virus type A are conjugated to amino acids on the surface of a carrier protein. Methods of making the conjugates and formulating vaccines are provided herein. The invention also provides for methods of vaccination of patients in which the patient achieves long term protection against disease and debilitating symptoms caused by infection with influenza virus type A.

Peptides

The extracellular portion of the M2 protein of influenza virus type A is generally recognized as the 24 N-terminal amino acids of the protein. The peptides used in the vaccine have an amino acid sequence chosen from within this 24 amino acid sequence. The particular sequence of the peptides can be the entire 24 amino acids sequence or a subset thereof having at least 7 amino acids and including an antigenic epitope.

It should be noted that the first amino acid of the M2 protein of influenza is a methionine. In any of the embodiments of the invention the presence of the terminal methionine is optional.

Effective subsequences of the 24 N-terminal amino acids can be determined, for example, through the following process. Initially, a peptide having the subsequence is tested to determine if it is bound by antibodies produced against the 24 amino acid sequence. The peptide is then conjugated to a carrier protein and the resulting conjugate is used to vaccinate an animal such as a mouse, ferret or monkey. Serum from the animal is tested for the presence of antibodies to the peptide. Finally, the animal is challenged with influenza virus. The course of the infection and the severity of the resulting disease are assessed. The process is best carried out with a number of animals and the results are assessed across all animals. If vaccination with the conjugate reduces the level of infection or the severity of the resulting disease then the peptide is considered useful in the preparation of a vaccine.

In preferred embodiments, the amino acid sequences of the peptides include the 24, 23, 22, 21, 20, 19, 18, 17, 16, 15, 14, etc., N-terminal amino acids of the M2 protein. The minimum size is limited only by the size of the epitope one desires to present to the immune system of a patient. Some preferred amino acid sequences are SEQ ID NOs: 1, 10 and 39.

| | |
|---|---|
| SEQ ID NO | Amino acid sequence |

| | |
|---|---|
| 1 | Ac-SLLTEVETPIRNEWGCRCNDSSD-Aha-C—NH2 (Aha = 6-aminohexanoic acid) |
| 2 | Ac-SLLTEVETPIRNEWGSRSNDSSD-Aha-C—NH2 |
| 3 | Ac-SLLTEVETPIRNEWGCRSNDSSD-Aha-C—NH2 |
| 4 | Ac-SLLTEVETPIRNEWGSRCNDSSD-Aha-C—NH2 |
| 5 | Ac-SLLTEVETPIRNEWGCRCNDSSDPL-MKQIEDKLEEILSKLYHIENELARIKKLLGER-NH-2 |
| 6 | Ac-MSLLTEVETPIRNEWGCRCNDSSDPLVVAASIIGILHLILWILD-NH2 |
| 7 | Ac-SLLTEVETPIRNEWGCRCNDSSDPLVVAAS-Aha-C—NH2 |
| 8 | Ac-SLLTEVETPIRNEWGC-(S-Acm)RC-(S-Acm)NDSSD-Aha-C—NH2 |
| 9 | C-b-SSLTEVETPIRNEWG-Abu-R-Abu-NDSSD |
| 10 | Ac-SLLTEVETPIRNEWG-Aha-C—NH2 |
| 11 | Bromoacetyl-Aha-SLLTEVETPIRNEWG-NH2 |
| 12 | 4-maleimidobutyryl-Aha-SLLTEVETPIRNEWG-NH2 |
| 13 | Ac-SLLTEVETPIRNEWG-Aha-Lys(Bromoacetyl)-NH2 |
| 14 | Ac-SLLTEVETPIRNEWG-Aha-Lys(4-maleimidobutyryl)-NH2 |
| 15 | CGPEKQTRGLFGAIAGFIENG |
| 16 | RVIEKTNEKFHQIEKEFSEVEGRIQDLEK |
| 17 | KIDLWSYNAELLVALENQHT |
| 18 | Ac-SLLTEVETPIRN-Aha-C—NH2 |
| 19 | Ac-SLLTEVETPIRNEW-Aha-C—NH2 |
| 20 | Ac-SLLTEVETPIRNE-Aha-C—NH2 |
| 21 | Ac-SLLTEVETPARNEWGSRSNDSSD-Aha-C—NH2 |
| 22 | Ac-SLLTEVETPIANEWGSRSNDSSD-Aha-C—NH2 |
| 23 | Ac-SLLTEVETPIRNEWGSRSNDSSD-Aha-K(4-maleimidobutyryl)-NH2 |
| 24 | Ac-LTEVETPIRNEW-NH2 |
| 25 | Ac-LTEVET-Aib-PIRNEW-NH2 |
| 26 | Ac-SLLTEVATPIRNEWGSRSNDSSD-NH2 |
| 27 | Ac-SLLTEAETPIRNEWGSRSNDSSD-NH2 |
| 28 | Ac-ALLTEVETPIRNEWGSRSNDSSD-NH2 |
| 29 | Ac-SLATEVETPIRNEWGSRSNDSSD-NH2 |
| 30 | Ac-SALTEVETPIRNEWGSRSNDSSD-NH2 |
| 31 | Ac-SLLTEVETPIRNEWASRSNDSSD-NH2 |
| 32 | Ac-SLLTEVETPIRNEWGSRSNDSSA-NH2 |
| 33 | Ac-SLLTEVETPIRNEWGSRSNDSAD-NH2 |
| 34 | Ac-SLLTEVETPIRNEWGSRSNDASD-NH2 |
| 35 | Ac-SLLTEVETPIRNEWGSRSNASSD-NH2 |
| 36 | Ac-SLLTEVETPIRNEWGSRSADSSD-NH2 |
| 37 | Ac-SLLTEVETPIRNEWGSRANDSSD-NH2 |
| 38 | Bromoacetyl-Aha-SLLTEVETPIRNEWGSRSNDSSD-NH2 |
| 39 | Ac-SLLTEVETPIRNEWGSRSNDSSD-Aha-Lys(BrAc)-NH2 |
| 40 | 4-Maleimidobutyryl-Aha-SLLTEVETPIRNEWGSRSNDSSD-NH2 |
| 41 | Ac-LTEVETPIRNEW-NH2 |
| 42 | Ac-SLLTEVETAIRNEWGSRSNDSSD-NH2 |
| 43 | Ac-SLLTEVET-Aib-IRNEWGSRSNDSSD-NH2 |
| 44 | Ac-SLLTEVEAPIRNEWGSRSNDSSD-NH2 |
| 45 | Ac-SLLTAVETPIRNEWGSRSNDSSD-NH2 |
| 46 | Ac-SLLAEVETPIRNEWGSRSNDSSD-NH2 |
| 47 | Ac-SLLTEVETPIRNEWGSASNDSSD-NH2 |
| 48 | Ac-SLLTEVETPIRNEWGARSNDSSD-NH2 |
| 49 | Ac-SLLTEVPIRNEWGSRSNDSSD-NH2 |
| 50 | Ac-SLLTEVETPARNEWGSRSNDSSD-NH2 |
| 51 | Ac-SLLTEVETPIRNEAGSRSNDSSD-NH2 |
| 52 | Ac-SLLTEVETPIRNAWGSRSNDSSD-NH2 |
| 53 | Ac-SLLTEVETPIRAEWGSRSNDSSD-NH2 |
| 54 | Ac-SLLTEVETPIANEWGSRSNDSSD-NH2 |
| 55 | Ac-Ser-Leu-Leu-Thr-Glu-Val-Glu-Thr-Pro-IIe-Arg-Asn-Glu-Trp-Gly-Asp- |
| | Arg-Ser-Asn-Asp-Ser-Ser-Asp-Aha-Cys-NH2 |
| 56 | Ac-Ser-Leu-Leu-Thr-Glu-Val-Glu-Thr-Pro-Ile-Arg-Asn-Glu-Trp-Gly-Dap- |
| | Arg-Ser-Asn-Asp-Ser-Ser-Asp-Aha-Cys-NH2 |
| 57 | Ac-Ser-Leu-Leu-Thr-Glu-Val-Glu-Thr-Pro-Ile-Arg-Asn-Glu-Trp-Gly-Asp- |
| | Arg-Ser-Asn-Asp-Ser-Ser-Asp-Aha-Cys-NH2 |
| 58 | Ac-Ser-Leu-Leu-Thr-Glu-Val-Glu-Thr-Pro-Ile-Arg-Asn-Glu-Trp-Gly-Dap- |
| | Arg-Ser-Asn-Asp-Ser-Ser-Asp-Aha-Cys-NH2 |

In embodiments wherein the amino acid sequence of the peptide includes the cysteine at position 17 or position 19 of the M2 protein, the cysteine may preferably be substituted with a serine. The substitution of serine for cysteine can be useful because, depending on the conjugation technique used, the reactivity of cysteine can lead to multimerization of the peptides, conjugation of peptide to peptide, or conjugation of the peptide to the carrier protein at the internal cysteines rather than at the added terminal cysteine of the peptide. These side reactions can result in lower peptide loading yields for the conjugate. However, it should be noted that conjugation of the peptide to the carrier protein at an internal cysteine of the peptide would not lead to an ineffective vaccine and is within the scope of this invention.

Certain segments of HA₀, particular those located in the intersubunit cleavage site region and in the HA₂ subunit, are highly conserved. Based on in vivo immunogenicity and protection studies with an extensive series of overlapping HA₀ peptides, we have identified several HA₀ regions containing protective epitopes. One region encompasses the cleavage site of HA₀ and the others are located in the HA₂ subunit (See table below).

Furthermore, the combination of a conjugate made with an HA peptide and a conjugate made with an M2 peptide was able to provide superior protection against diseases caused by influenza type A as compared either conjugate given alone. Therefore, one preferred embodiment of this invention is a vaccine containing a M2 peptide conjugate in combination with conjugates composed of other conserved, protective influenza virus peptides. A preferred embodiment of a method of this invention is the administration of such a vaccine to a patient wherein the patient develops an immunological response against influenza type A that is superior to the immunological response seen upon administration of a vaccine having only a M2 peptide conjugate.

HA peptides can be chosen from the following:

| | | |
|---|---|---|
| SEQ ID | | |
| NO | Short Name | Sequence |

Influenza A

| | | |
|---|---|---|
| 59 | Cys-A/H3/HA2-6 | CbKIDLWSYNAELLVALENQHT-NH2 |
| 63 | A/H3/HA2-9-Cys | GLFGAIAGFIENGWEGMIDGGCGKKKK-NH2 |
| 64 | Cys-A/H3/HA2-10 | CbIEKTNEKFHQIEKE-NH2 |
| 65 | Cys-A/H3/HA2-11 | CbRVIEKTNEKFHQIEKEFSEVEGRIQDLEKYVEDTK-NH2 |
| 66 | A/H3/HA2-12-Cys | IEKEFSEVEGRIQDLEKYVEDTKbC-NH2 |
| 67 | A/H3/HA2-13-Cys | Ac- |
| | | DQINGKLNRVIEKTNEKFHQIEKEFSEVEGRIQDLEKYVEDTKIDLWSYNAELLVALE |
| | | NQHTIDLKGGC-NH2 |
| 68 | A/H3/HA2-15 | Ac- |
| | | CGGDQINGKLNRVIEKTNEKFHQIEKEFSEVEGRIQDLEKYVEDTKIDLWSYNAELL |
| | | VALENQHTIDLKGGC-NH2 |
| 69 | Cys-A/H3/HA2-16 | CbRTRKQLRENAEDMGNGAbuFKIY-NH2 |
| 70 | Cys-A/H3/HA2-17 | Ac-CGGRIQDLEKYVEDTKIDLWSYNAELLVALENQHT-NH2 |
| 71 | Cys-A/H3/HA2-19 | CGWYGFRHQNSEGTGQAADLK-NH2 |
| 72 | A/H3(L)/HA2-20-Cys | GLFGAIAGFIENGCE-OH |
| 73 | A/H3(L)/HA2-22-Cys | Ac-GLFGAIAGFIENGCE-OH |
| 74 | A/H3(L)/HA2-23-Cys | Suc-GLFGAIAGFIENGCE-OH |
| 75 | Cys-A/H3(L)/HA2-21 | Ac-CGGLFGAIAGFIENGE-OH |
| 76 | A/H3(L)/HA2-24-Cys | Ac-GLFGAIAGFIENGWEGMVDGCE-OH |
| 77 | A/H3(L)/HA2-25-Cys | GLFGAIAGFIENGWEGMVDGCE-OH |
| 78 | Cys-A/H3(L)/HA2-26 | Ac-CGQTRGLFGAIAGFIENGE-OH |
| 79 | A/H3/HA2-25-Cys | GIFGAIAGFIENGWEGMVDGCE-OH |
| 80 | A/H1/HA2-25-Cys | GLFGAIAGFIEGGWTGMIDGCE-OH |
| 81 | A/H3(L)/HA2-26-Cys | GLFGAIAGFIENGWEGMVDGKKCE-OH |
| 82 | A/H1/HA2-26-Cys | GLFGAIAGFIEGGWTGMIDGKKCE-OH |
| 83 | Cys-A/H3/HA0-2 | CGPEKQTRGLFGAIAGFIENG-NH2 |
| 84 | A/H3I/A0-4-Cys | PEKQTRGLFGAIAGFIGluNGGCGKKKK-NH2 (Pro-Glu lactam bridge) |
| 85 | Cys-A/H3/HA0-7 | PEKQTRGLFGAIAGFIC (cyclic) |
| 86 | Cys-A/H3/HA0-8 | CGPEKQTRGLFGA-NH2 |
| 87 | A/H3/HA0-9-Cys | PEKQTRGLFGAIAGFIENGC-NH2 |
| 88 | A/H3/HA0-10-Cys | GMRNVPEKQTRGLFGAIAGFIENGC-NH2 |
| 89 | A/H3/HA0-11 | CGPEKQTRGLFG-NH2 |
| 90 | A/H3/HA0-12 | CGPEKQTRGLF-NH2 |
| 91 | A/H3/HA0-13 | CGPEKQTRGL-NH2 |
| 92 | A/H3/HA0-14 | CGPEKQTRG-NH2 |
| 93 | A/H3/HA0-15 | CGMRNVPEKQTRGLFGAIAGFIENG-NH2 |
| 94 | A/H3/HA0-16 | CGNVPEKQTRGLFGAIAGFIENG-NH2 |
| 95 | Ac-A/H3/HA0-11 | Ac-CGPEKQTRGLFG-NH2 |
| 96 | Ac-A/H3/HA0-12 | Ac-CGPEKQTRGLF-NH2 |
| 97 | Ac-A/H3/HA0-13 | Ac-CGPEKQTRGL-NH2 |
| 98 | Ac-A/H3/HA0-14 | Ac-CGPEKQTRG-NH2 |
| 99 | Ac-A/H3/HA0-15 | Ac-CGMRNVPEKQTRGLFGAIAGFIENG-NH2 |
| 100 | Ac-A/H3/HA0-16 | Ac-CGNVPEKQTRGLFGAIAGFIENG-NH2 |
| 101 | Ac-A/H3/HA0-2 | Ac-CGPEKQTRGLFGAIAGFIENG-OH |
| 102 | Cys-A/H3/HA0-18 | Ac-CGPEKQTRGLFGAIAGFIENGE-OH |
| 103 | Cys-A/H3/HA0-19 | Suc-CGPEKQTRGLFGAIAGFIENGE-OH |
| 104 | A/H3/HA0-17-Cys | Suc-EPEKQTRGLFGAIAGFIENGC-OH |
| 105 | BrAc-A/H3(L)/HA0-2 | BrAc-GPEKQTRGLFGAIAGFIENG-NH2 |
| 106 | BrAc-NH1/HA0-2 | BrAc-GPSIQSRGLFGAIAGFIEGG-NH2 |
| 107 | Cys-A/H1/HA0-2 | CGPSIQSRGLFGAIAGFIEGG-NH2 |
| 108 | Cys-A/H3/HA0-20 | CGPEKQTRGIFGAIAGFIENG-NH2 |
| 109 | BrAc-A/H3/HA0-21 | BrAc-GPEKQTRGIFGAIAGFIEE-OH |
| 110 | BrAc-A/H3/HA0-22 | BrAc-EGPEKQTRGIFGAIAGFIEE-OH |
| 111 | BrAc-A/H1/HA0-21 | BrAc-GPSIQSRGLFGAIAGFIEE-OH |
| 112 | BrAc-A/H1/HA0-22 | BrAc-EGPSIQSRGLFGAIAGFIEE-OH |
| 113 | Cys-A/H3/HA0-22 | Ac-CEGPEKQTRGIFGAIAGFIEE-OH |
| 114 | Cys-A/H1/HA0-21 | Ac-CGPSIQSRGLFGAIAGFIEE-OH |
| 115 | Cys-A/H1/HA0-22 | Ac-CEGPSIQSRGLFGAIAGFIEE-OH |
| 116 | Cys-A/H3(L)/HA0-24 | Ac-CEGPEKQTRGLFGAIAGFIENGWEGMIDE-OH |
| 62 | Cys-A/H3(L)/HA0-25 | Ac-CEGMRNVPEKQTRGLFGAIAGFIENGE-OH |
| 117 | Mal-A/H1/HA0-21 | Mal-GPSIQSRGLFGAIAGFIEE-OH |
| 118 | Cys-A/H3(L)/HA0-22 | Ac-CEGPEKQTRGLFGAIAGFIEE-OH |
| 119 | Cys-A/H1/HA0-27 | Ac-CRGLFGAIAGFIEGGWTGMIDGE-OH |
| 61 | Cys-A/H1/HA0-25 | Ac-CEGLRNIPSIQSRGLFGAIAGFIEGGE-OH |
| 120 | Cys-A/H1/HA0-28 | Ac-CEGLRNIPSIQSRGLFGAIAGFIEGGWTGMIDGE-OH |
| 121 | Cys-A/H1/HA0-29 | Ac-CRGLFGAIAGFIEGGWTGMIDGKKE-OH |
| 122 | Cys-A/H1/HA0-30 | Ac-CEGLRNIPSIQSRGLFGAIAGFIEGGWTGMIDGKKE-OH |
| 123 | Cys-A/H1/HA0-31 | Ac-CEGLRNIPSIQSRGLE-OH |
| 124 | BrAc-A/H3(L)/HA0-25 | BrAc-Ahx-EGMRNVPEKQTRGLFGAIAGFIENGE-OH |
| 125 | BrAc-A/H1/HA0-25 | BrAc-Ahx-EGLRNIPSIQSRGLFGAIAGFIEGGE-OH |

Influenza B

| | | |
|---|---|---|
| 126 | BrAc-B/HA0-21 | BrAc-GPAKLLKERGFFGAIAGFLEE-OH |
| 127 | Cys-B/HA0-21 | Ac-CGPAKLLKERGFFGAIAGFLEE-OH |
| 60 | BrAc-B/HA0-22 | BrAc-EGPAKLLKERGFFGAIAGFLEE-OH |
| 128 | Cys-B/HA0-22 | Ac-CEGPAKLLKERGFFGAIAGFLEE-OH |
| 129 | BrAc-B/HA0-23 | BrAc-EGAKLLKERGFFGAIAGFLEE-OH |
| 130 | BrAc-Ahx-B/HA0-22 | BrAc-Ahx-EGPAKLLKERGFFGAIAGFLEE-OH |
| 131 | Mal-Ahx-B/HA0-22 | Mal-Ahx-EGPAKLLKERGFFGAIAGFLEE-OH |
| 132 | Cys-Ahx-B/HA0-22 | Cys-Ahx-EGPAKLLKERGFFGAIAGFLEE-OH |
| 133 | Ac-B/HA0-22 | Ac-EGPAKLLKERGFFGAIAGFLEE-OH |
| 134 | B/HA0-22-E1 | Ac-GPAKLLKERGFFGAIAGFLE-NH2 |
| 135 | B/HA0-22-N1 | Ac-AKLLKERGFFGAIAGFLE-NH2 |
| 136 | B/HA0-22-N2 | Ac-KLLKERGFFGAIAGFLE-NH2 |
| 137 | B/HA0-22-N3 | Ac-LLKERGFFGAIAGFLE-NH2 |
| 138 | B/HA0-22-N4 | Ac-LKERGFFGAIAGFLE-NH2 |
| 139 | B/HA0-22-N5 | Ac-KERGFFGAIAGFLE-NH2 |
| 140 | B/HA0-22-N6 | Ac-ERGFFGAIAGFLE-NH2 |
| 141 | B/HA0-22-N7 | Ac-RGFFGAIAGFLE-NH2 |
| 142 | B/HA0-22-N8 | Ac-GFFGAIAGFLE-NH2 |
| 143 | B/HA0-22-C1 | Ac-GPAKLLKERGFFGAIAGFL-NH2 |
| 144 | B/HA0-22-C2 | Ac-GPAKLLKERGFFGAIAGF-NH2 |
| 145 | B/HA0-22-C3 | Ac-GPAKLLKERGFFGAIAG-NH2 |
| 146 | B/HA0-22-C4 | Ac-GPAKLLKERGFFGAIA-NH2 |
| 147 | B/HA0-22-C5 | Ac-GPAKLLKERGFFGAI-NH2 |
| 148 | B/HA0-22-C6 | Ac-GPAKLLKERGFFGA-NH2 |
| 149 | B/HA0-22-C7 | Ac-GPAKLLKERGFFG-NH2 |
| 150 | B/HA0-22-C8 | Ac-GPAKLLKERGFF-NH2 |
| 151 | B/HA0-22-C9 | Ac-GPAKLLKERGF-NH2 |
| 152 | B/HA0-22-C10 | Ac-GPAKLLKERG-NH2 |
| 153 | B/HA0-22-C11 | Ac-GPAKLLKER-NH2 |
| 154 | BrAc-Ahx-B/HA0-22-A1 | BrAc-Ahx-AGPAKLLKERGFFGAIAGFLEE-OH |
| 155 | BrAc-Ahx-B/HA0-22-A3 | BrAc-Ahx-EGAAKLLKERGFFGAIAGFLEE-OH |
| 156 | BrAc-Ahx-B/HA0-22-A4 | BrAc-Ahx-EGPAALLKERGFFGAIAGFLEE-OH |
| 157 | BrAc-Ahx-B/HA0-22-A5 | BrAc-Ahx-EGPAKALKERGFFGAIAGFLEE-OH |
| 158 | BrAc-Ahx-B/HA0-22-A6 | BrAc-Ahx-EGPAKLAKERGFFGAIAGFLEE-OH |
| 159 | BrAc-Ahx-B/HA0-22-A7 | BrAc-Ahx-EGPAKLLAERGFFGAIAGFLEE-OH |
| 160 | BrAc-Ahx-B/HA0-22-A8 | BrAc-Ahx-EGPAKLLKARGFFGAIAGFLEE-OH |
| 161 | BrAc-Ahx-B/HA0-22-A9 | BrAc-Ahx-EGPAKLLKEAGFFGAIAGFLEE-OH |
| 162 | BrAc-Ahx-B/HA0-22-A12 | BrAc-Ahx-EGPAKLLKERGAFGAIAGFLEE-OH |
| 163 | BrAc-Ahx-B/HA0-22-A13 | BrAc-Ahx-EGPAKLLKERGFAGAIAGFLEE-OH |
| 164 | BrAc-Ahx-B/HA0-22-A16 | BrAc-Ahx-EGPAKLLKERGFFGAAAGFLEE-OH |
| 165 | BrAc-Ahx-B/HA0-22-A19 | BrAc-Ahx-EGPAKLLKERGFFGAIAGALEE-OH |
| 166 | BrAc-Ahx-B/HA0-22-A20 | BrAc-Ahx-EGPAKLLKERGFFGAIAGFAEE-OH |
| 167 | BrAc-Ahx-B/HA0-22-A21 | BrAc-Ahx-EGPAKLLKERGFFGAIAGFLAE-OH |
| 168 | BrAc-Ahx-B/HA0-22-A22 | BrAc-Ahx-EGPAKLLKERGFFGAIAGFLEA-OH |

BrAc = bromoacelyl
Ac = acetyl
Mal = maleimidyl
Suc = succinyl
Ahx = 6-aminohexanoic acid
b = beta-alanine
Abu = 2-aminobutyric acid

Furthermore, the combination of a conjugate made with the influenza type B HA₀ cleavage site peptide and a conjugate made with an influenza type A M2 peptide was able to provide protection against diseases caused by both influenza type A and influenza type B. Therefore, one preferred embodiment of this invention is a vaccine containing a M2 peptide conjugate in combination with conjugates composed of other conserved, protective peptides from influenza type B. A further preferred embodiment of this invention is a vaccine containing a M2 peptide conjugate in combination with conjugates composed of other conserved, protective peptides from influenza type A and with conjugates composed of other conserved, protective peptides from influenza type B. A preferred embodiment of a method of this invention is the administration of such a vaccine to a patient wherein the patient develops an immunological response against influenza type A that is superior to the immunological response seen upon administration of a vaccine having only a M2 peptide conjugate.

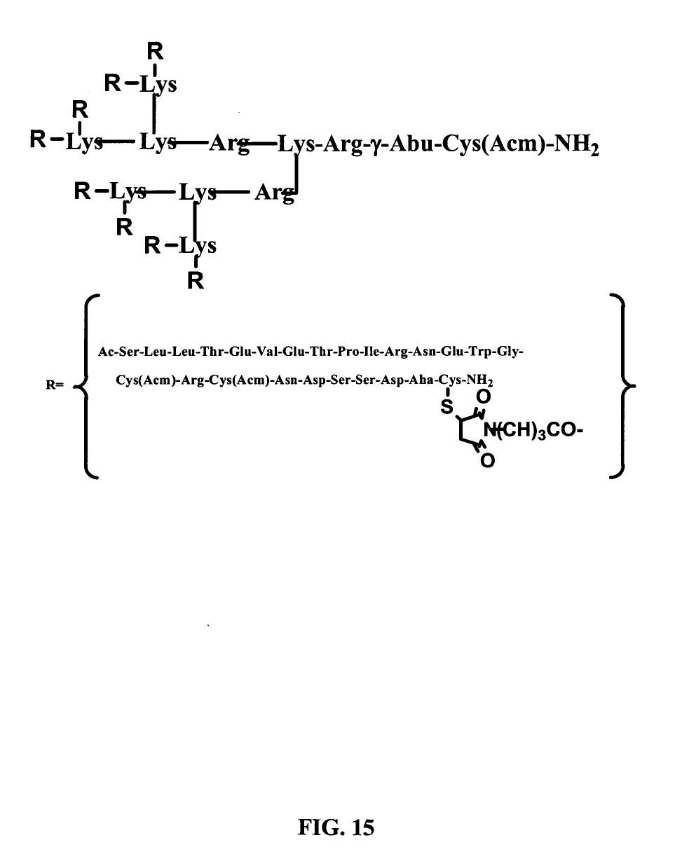

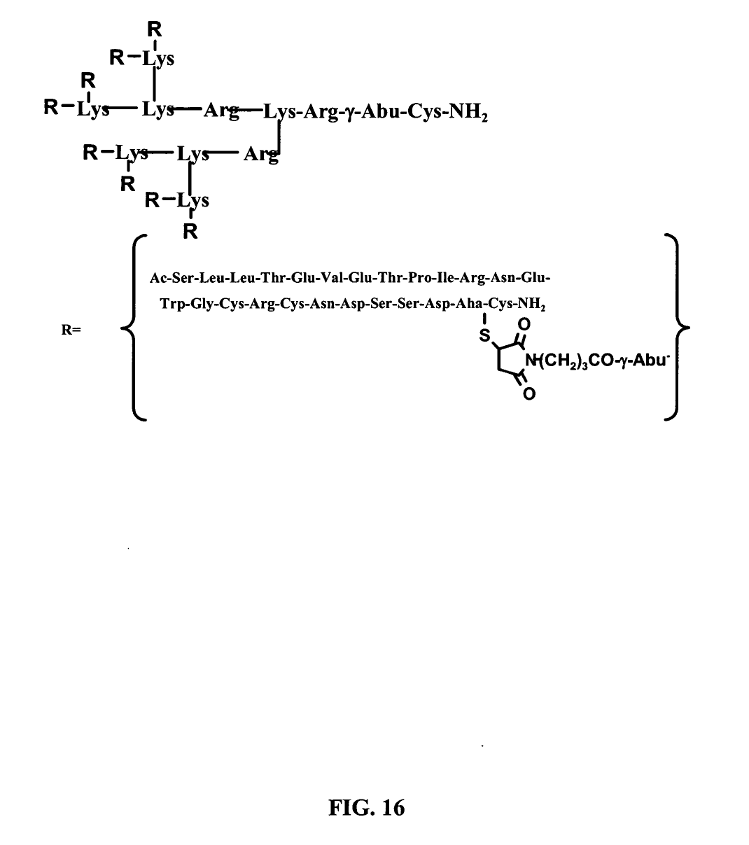

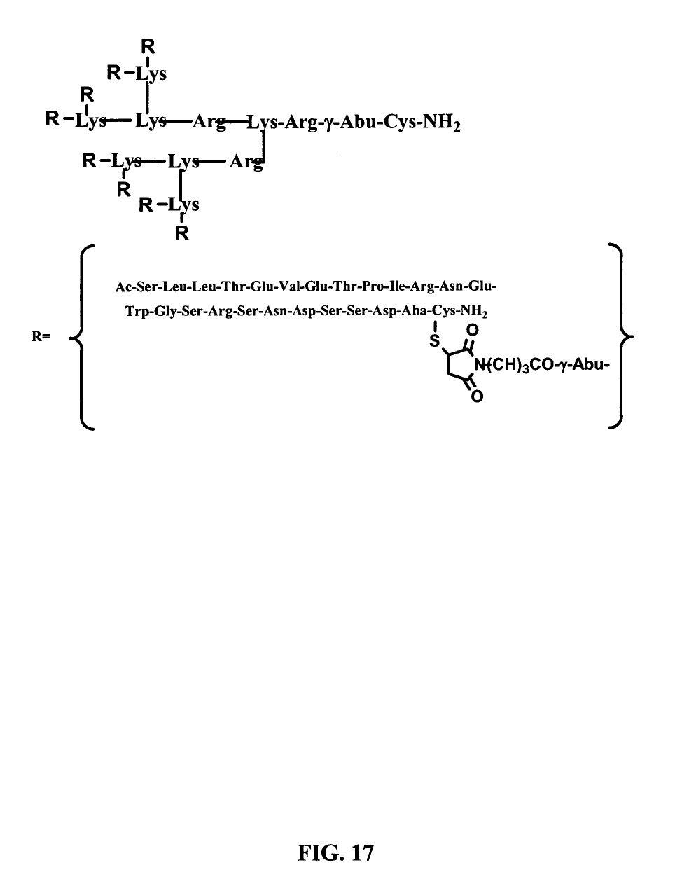

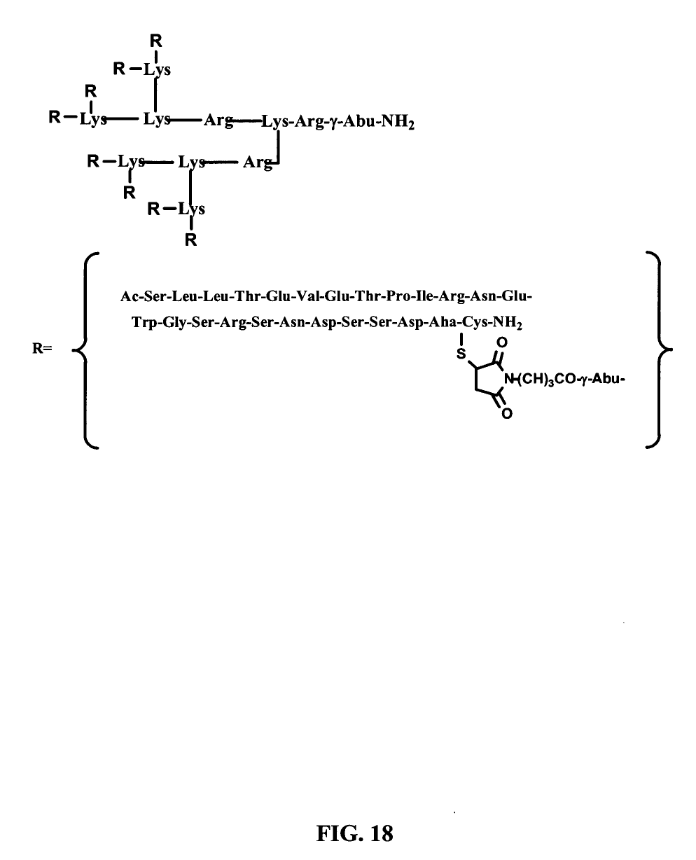

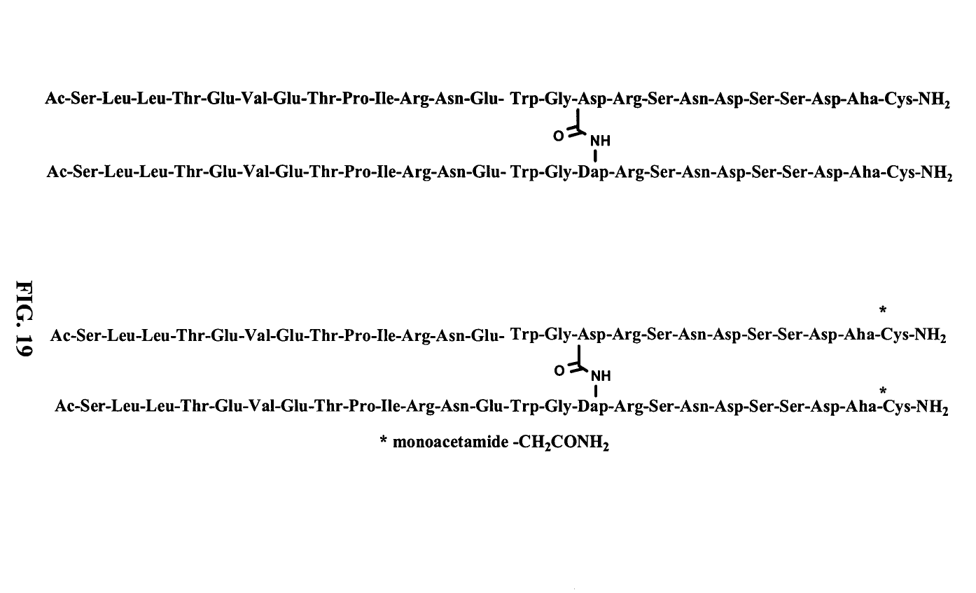

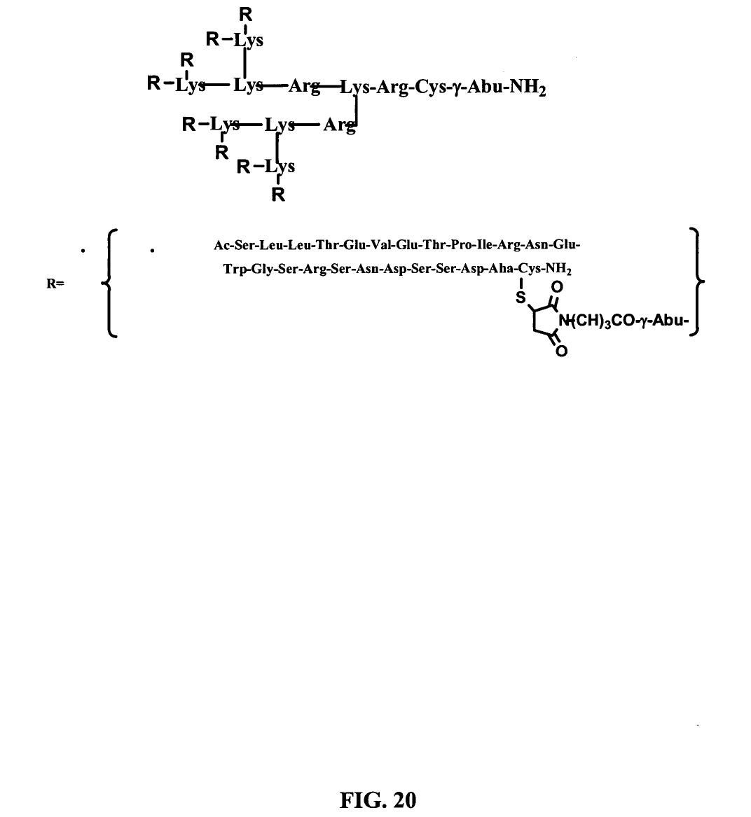

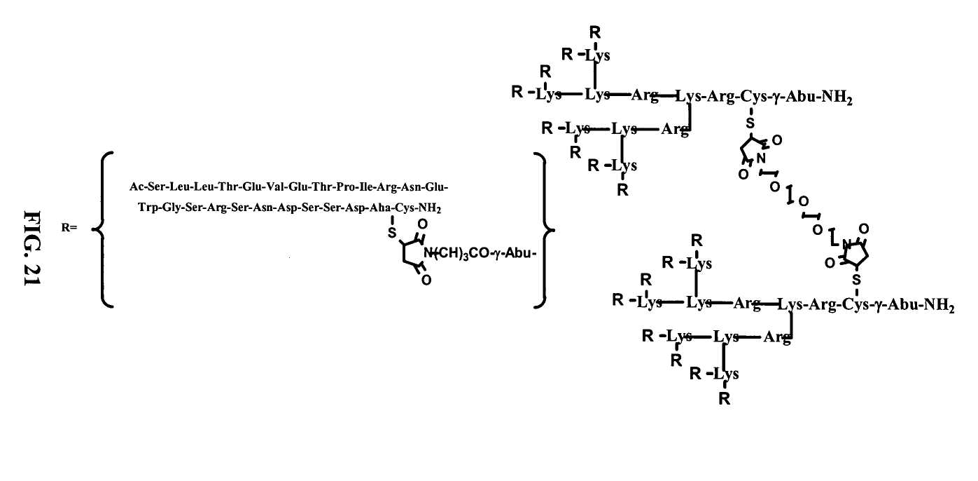

M2 or HA₀ peptide antigens can also be represented by multiple antigenic peptides (MAPs) on a lysine or other suitable scaffold. Peptides arrayed in such a manner can be used in the conjugate vaccines of this invention. Examples can be seen in FIGS. 15-18 & 20-21. Another alternative presentation of peptides in conjugates vaccines of this invention are dimeric M2 or HA₀ peptides. In this format, a linking bond, preferably covalent, is used to cross-link two peptides to form a dimer. Examples for M2 peptides can be seen in FIG. 19. Conjugate vaccines in which the peptides are arrayed in this manner can be more antigenic than vaccines made with the corresponding monomeric peptide conjugates.

Peptides can be produced using techniques well known in the art. Such techniques include chemical and biochemical synthesis. Examples of techniques for chemical synthesis of peptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, 1987-1998, and Sambrook, et al., in Molecular Cloning, A Laboratory Manual, 2nd Edition, Cold Spring Harbor Laboratory Press, 1989.

Carrier Proteins

A carrier protein, as referred to herein, means an immunogenic protein to which the peptides are conjugated. Various carrier proteins are known in the art and have been used in polysaccharide-protein conjugate vaccines. These and other immunogenic proteins can also be used in vaccines of this invention. Preferred carrier proteins are the outer membrane protein complex of Neiserria meningitidis (OMPC), tetanus toxoid protein, Hepatitis B virus proteins including the Surface antigen protein (HBsAg) and the Core Antigen protein (HB Core), keyhole limpet hemocyanin (KLH), rotavirus capsid proteins and the L1 protein of a bovine Pappiloma virus VLP or human Papilloma Virus VLP, for example, VLPs of HPV type 6, 11 or 16, etc.

For ease of manufacture, one can use a single type of carrier protein to make a conjugate. However, one can also prepare more than one conjugate using a different carrier protein in each one. Then, one can mix the conjugates when formulating the vaccine. In this manner one can provide a vaccine which, in addition to generating an immune response against influenza, also produces an immune response against the different carrier proteins used in the conjugates. Further permutations of conjugates combining various peptides and carrier proteins are also possible, if desired.

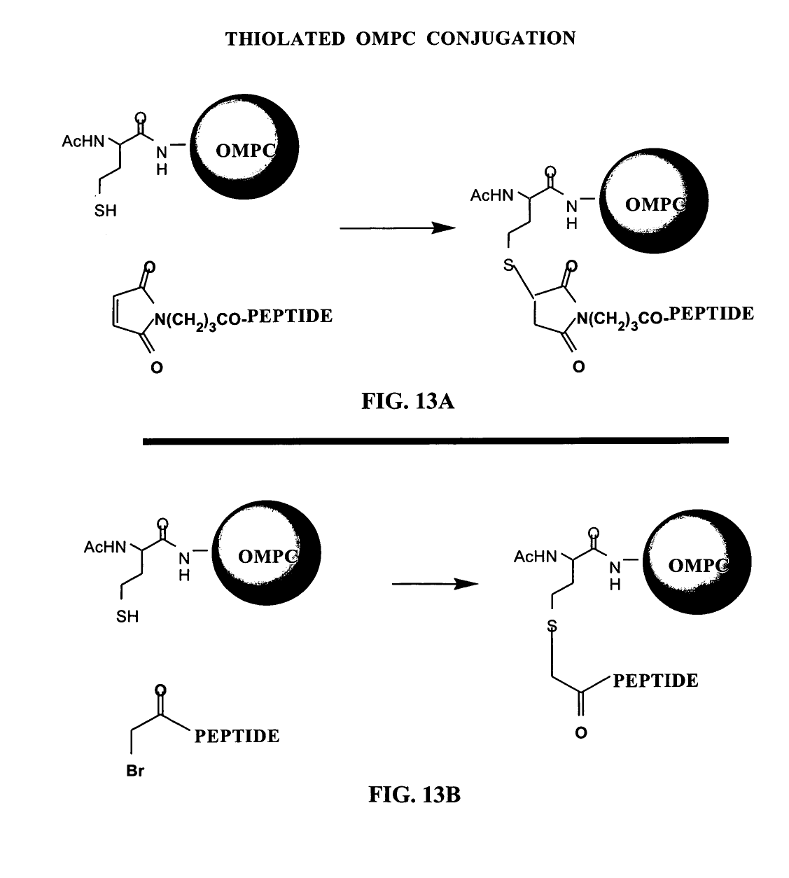

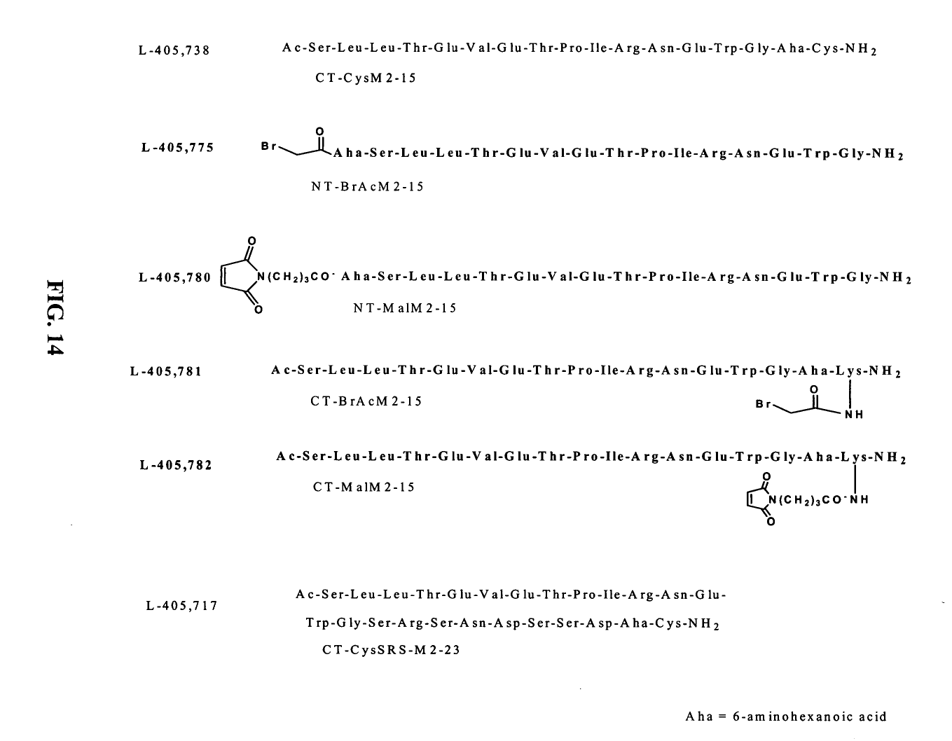

A preferred carrier protein is OMPC. OMPC contains numerous reactive sites available for conjugation. The availability of a reactive site for conjugation is determined by the grouping of atoms present and the position of the group in OMPC. Nucleophilic functionalities available for conjugation can be determined using techniques well know in the art. (See Emini, et al. U.S. Pat. No. 5,606,030.) One type of group that can be used as a reactive site for conjugation is primary amino groups present on amino acids such as the epsilon amino group of lysine and the alpha amino group of N-terminal amino acids of proteins. In addition, conversion of these amino groups to give the thiolated form of OMPC provides a reactive functionality which may be used for conjugation to thiol reactive peptides. Examples of thiol reactive peptides are bromoacetylated or maleimide derivatized peptides as illustrated in FIG. 13. OMPC can be obtained using techniques well known in the art such as those described by Fu, U.S. Pat. No. 5,494,808.

Another preferred category of carrier proteins is represented by virus capsid proteins that have the capability to self-assemble into virus-like particles (VLPs). Examples of VLPs used as peptide carriers are hepatitis B virus surface antigen (HBsAg) and core antigen (HBcAg) (Pumpens et al., “Evaluation of HBs, HBc, and frCP virus-like particles for expression of human papillomavirus 16 E7 oncoprotein epitopes”, Intervirology, Vol. 45, pp. 24-32, 2002), hepatitis E virus particles (Niikura et al., “Chimeric recombinant hepatitis E virus-like particles as an oral vaccine vehicle presenting foreign epitopes”, Virology, Vol. 293, pp. 273-280, 2002), polyoma virus (Gedvilaite et al., “Formation of Immunogenic Virus-like particles by inserting epitopes into surface-exposed regions of hamster polyomavirus major capsid protein”, Virology, Vol. 273, pp. 21-35, 2000), and bovine papilloma virus (Chackerian et al., “Conjugation of self-antigen to papillomavirus-like particles allows for efficient induction of protective autoantibodies”, J. Clin. Invest., Vol. 108 (3), pp. 415-423, 2001). More recently, antigen-presenting artificial VLPs were constructed to mimic the molecular weight and size of real virus particles (Karpenko et al., “Construction of artificial virus-like particles exposing H[V epitopes and the study of their immunogenic properties”, Vaccine, pp. 386-392, 2003).

A suspected advantage of using papillomavirus VLPs as peptide antigen carrier is that it allows the presentation of antigenic sequence in an ordered array that is thought to ensure an optimal response from the immune system. In one report, exposure of the antigenic sequence in a matrix that mimics an icosahedral virion was found to abrogate the ability of the humoral immune system to distinguish between self and foreign (Chackerian et al., “Induction of autoantibodies to mouse CCR5 with recombinant papillomavirus particles”, Proc. Natl. Acad. Sci. USA, Vol. 96, pp. 2373-2378, 1999). By linking mouse self-peptide TNF-α to papilloma virus VLPs high-titers, long-lasting autoantibodies were induced in mice. One of the challenges in using VLPs as minimal antigen carriers is to avoid the decrease in immunogenicity of the developed conjugate vaccine due to the presence of anti-carrier antibodies induced by pre-exposure to the VLP carrier.

The human papillomavirus (HPV) VLPs possess a typical icosahedral lattice structure about 60 nm in size and each is formed by the assembly of 72 L1 protein pentamers (called capsomeres) (Chen et al., 2000; Modis et al., “Atomic model of the papilloma virus capsid”, EMBO J., Vol. 21, pp. 47544762, 2002). Bovine papillomavirus VLPs have been used successfully to carry an antigenic sequence either inserted by genetic fusion into the L1 protein (Chackerian et al., 1999), or L2 (Greenstone et al., “Chimeric papillomavirus virus-like particle elicit antitumor immunity against the E7 oncoprotein in an HPV 16 tumor model”, Proc. Natl. Acad. Sci. USA, Vol. 95, pp. 1800-1805, 1998) proteins of the VLPs or fused to streptavidin which then is bound to biotinylated VLPs (Chackerian et al., 2001).

The preparation of human and bovine papilloma virus VLPs is well known in the art as indicated by the references cited above and the following exemplary patents and patent publications: U.S. Pat. No. 6,159,729, U.S. Pat. No. 5,840,306, U.S. Pat. No. 5,820,870 and WO 01/14416.

Examples below describe the preparation and the immunogenicity of exemplary conjugate vaccines obtained by chemically conjugating peptide fragments of influenza to the human papillomavirus (HPV) virus-like particle (VLP). The resulting conjugate molecules, comprised of approximately 800 to 4,000 copies of the antigenic peptide per VLP, were obtained by reacting a C-terminal cysteine residue on the peptides and maleimide-activated HPV VLPs. These conjugates have an average particle size slightly larger than the VLP carrier alone and show enhanced overall stability against chemical and thermal-induced denaturation. The M2-HPV VLP conjugates lost the binding affinity for some anti-HPV conformational antibodies but are fully recognized by anti-M2 antibodies. An influenza M2 peptide-HPV VLP conjugate vaccine was formulated with aluminum adjuvant. Two doses of 30-ng peptide were found to be highly immunogenic and conferred good protection against lethal challenge of influenza virus in mice. These results indicate that HPV VLP can be used as a carrier for influenza peptides in conjugate vaccines.

Using the human papillomavirus VLP system as an antigen carrier for developing chemically coupled influenza peptide conjugate vaccines provides certain advantages. The chemical coupling avoids the potential problems of peptide insertion into the L1 sequence that can interfere with the proper assembly of the VLPs and is much simpler than the biotinylation and binding procedure. Moreover, the results presented show that chemical coupling allows much higher peptide loads per VLP compared to previously reported procedures. Moreover, in the Examples below, the peptide conjugation process did not induce significant alteration in the morphology of HPV VLPs. Therefore, VLPs, including HPV VLPs and the similar bovine papilloma virus VLPs, can be used to construct vaccines within this invention.

Conjugation

The peptides and the carriers of the present invention can be conjugated using any conjugation method in the art. For example, the conjugation can be achieved using sulfosuccinimidyl 4-(N-maleimidomethyl)cyclohexane-1-carboxylate (sSMCC), N-[ε-maleimidocaproyloxy]sulfosuccinimde ester (sEMCS), N-maleimidobenzoyl-N-hydroxysuccinimide ester (MBS), glutaraldehyde, 1-ethyl-3-(3-dimethylaminopropyl)carbodiimide (EDCI), Bis-diazobenzidine (BDB), or N-acetyl homocysteine thiolactone (NAHT).

In the carrier maleimide-activation method, the conjugation is achieved using sulfosuccinimidyl 4-(N-maleimidomethyl)cyclohexane-1-carboxylate (sSMCC), or N-maleimidobenzoyl-N-hydroxysuccinimide ester (MBS). The method using sSMCC is widely used and highly specific (See, e.g., Meyer et al. 2002, J. of Virol. 76, 2150-2158). sSMCC cross-links the SH-group of a cysteine residue to the amino group of a lysine residue on the carrier protein.

In the conjugation reaction using sSMCC, the carrier is first activated by binding the sSMCC reagent to the amine (e.g.: lysine) residues of the carrier. After separation of the activated carrier from the excess reagent and the by-product, the cysteine-containing peptide is added and the link takes place by addition of the SH-group to the maleimide function of the activated carrier. The method using MBS conjugates the peptide and the carrier through a similar mechanism.

The conjugation using sSMCC can be highly specific for SH-groups. Thus, cysteine residue in the peptide is essential for facile conjugation. If a peptide does not have a cysteine residue, a cysteine residue should be added to the peptide, preferably at the N-terminus or C-terminus. If the desired epitope in the peptide contains a cysteine, the conjugation should be achieved with a method not using a sSMCC activated carrier. If the peptide contains more than one cysteine residue, the peptide should not be conjugated to the carrier using sSMCC unless the excess cysteine residue can be replaced or modified.

The linkage should not interfere with the desired epitope in the peptide. The cysteine is preferably separated from the desired epitope sequence with a distance of at least one amino acid as a spacer.

Another conjugation useful in the present invention is achieved using N-acetyl homocysteine thiolactone (NAHT). For example, thiolactones can be used to introduce a thiol functionality onto OMPC, to allow conjugation with maleimidated or Bromo-acetylated-peptides (Tolman et al. Int. J. Peptide Protein Res. 41, 1993, 455-466; Conley et al. Vaccine 1994, 12, 445-451).

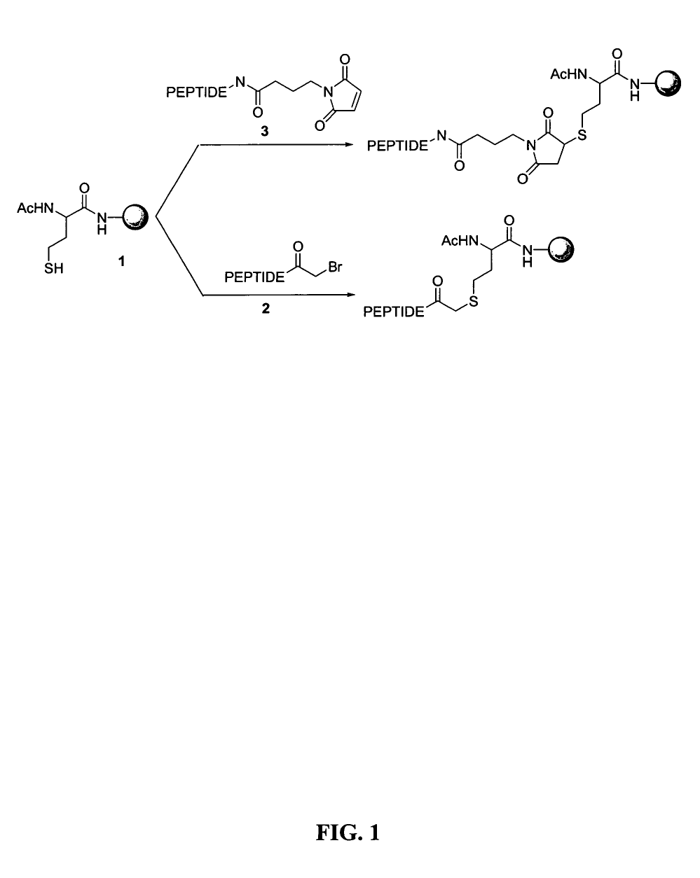

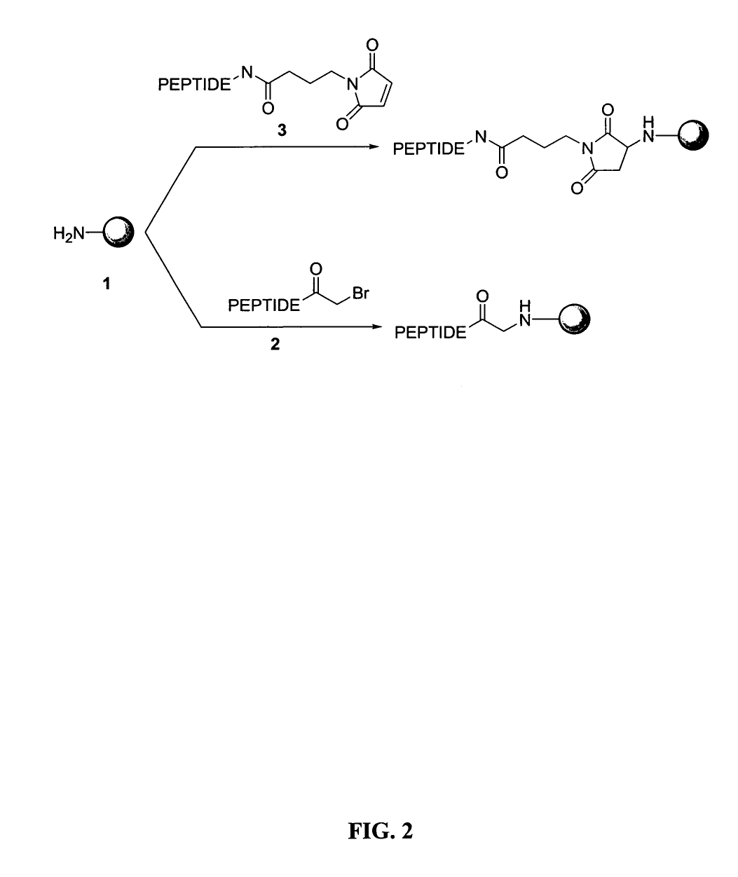

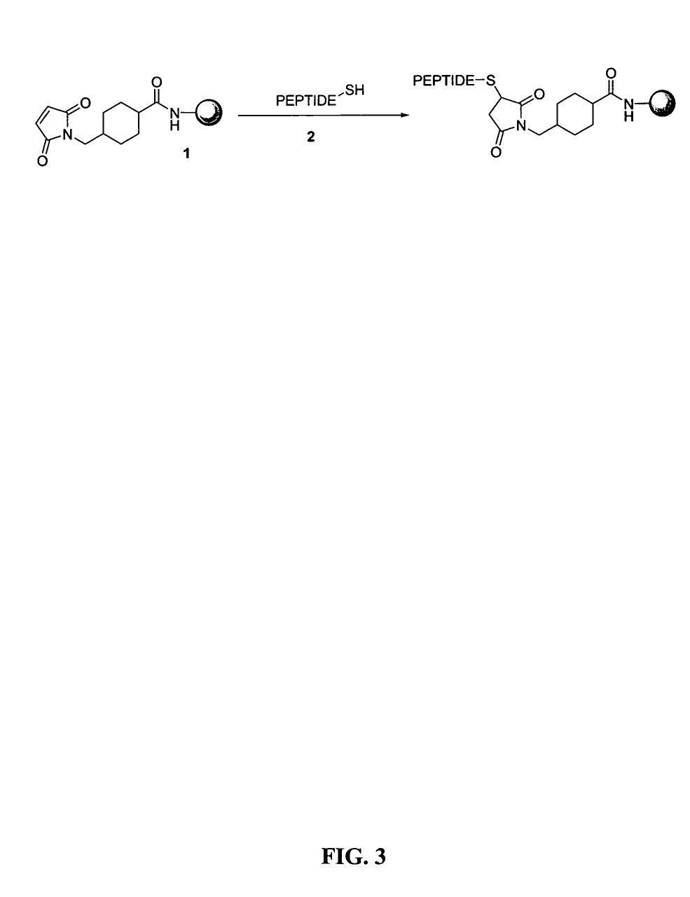

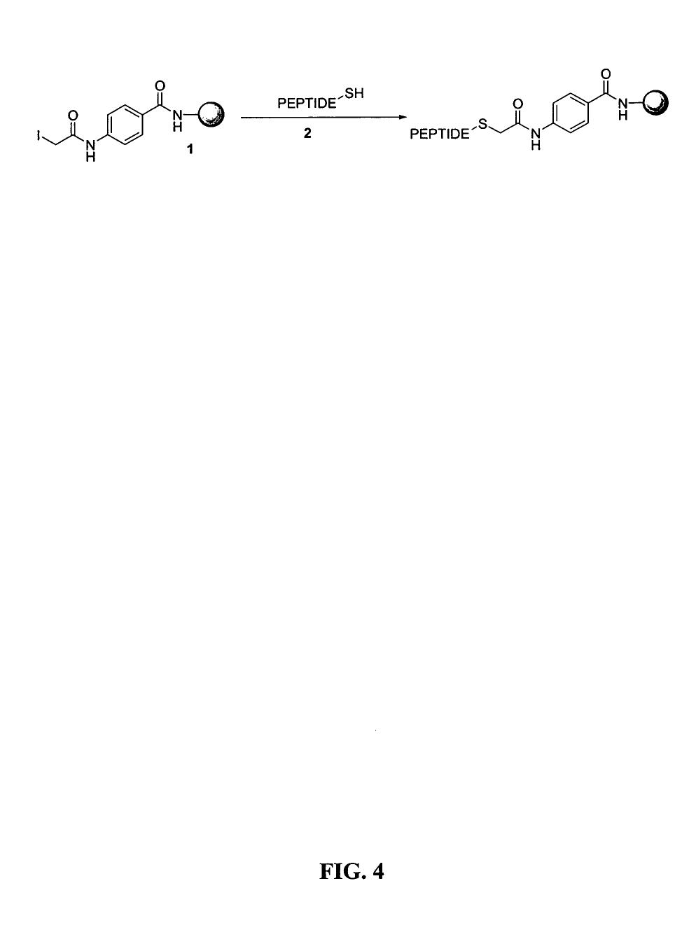

In particular embodiments of the invention, conjugation reactions to couple the peptide to the carrier protein involve introducing and/or using intrinsic nucleophilic groups on one reactant and introducing and/or using intrinsic electrophilic groups in the other reactant. A preferred activation scheme (I) (FIG. 1) would be to introduce a nucleophilic thiol group to the carrier protein (preferably OMPC) and adding electrophilic groups (preferably alkyl halides or maleimide) to the peptide. The resulting conjugate will have thiol ether bonds linking the peptide and carrier. Direct reaction of the peptide electrophilic group (maleimide or alkyl halide) and intrinsic nucleophilic groups (preferably primary amines or thiols) of the carrier protein, leading to secondary amine linkages (scheme (II) FIG. 2) or thio]ether bonds. However, the expected higher reactivity of the thiol nucleophile over the amine under similar reaction conditions would make scheme I preferable. Alternative schemes involve adding a maleimide group (III) FIG. 3 or alkyl halide (IV) FIG. 4 to the carrier and introducing a terminal cysteine to the peptide and/or using intrinsic peptide thiols again resulting in thiol ether linkages.

Linkage

A sulfur containing amino acid contains a reactive sulfur group. Examples of sulfur containing amino acids include cysteine and non-protein amino acids such as homocysteine. Additionally, the reactive sulfur may exist in a disulfide form prior to activation and reaction with carrier. Cysteines 17 and 19 present in the M2 sequence can be used in coupling reactions to a carrier activated with electrophilic groups such as maleimide or alkyl halides (Schemes III (FIG. 3) and IV (FIG. 4)). Introduction of maleimide groups using heterobifunctional cross-linkers containing reactive maleimide and activated esters is common. Attempts to achieve high levels of maleimide activation for multimeric protein can lead to cross-linking reactions in which amine groups can react with both functional groups of the cross-linker. This could result in lower levels of available maleimide groups and hence lower peptide loading. The cross-linking of subunits of a multimeric carrier could also effect the immunogenicity and/or stability of the conjugate. For peptides having multiple cysteines, multiple links with the carrier maleimide or alkylhalide groups can occur with a single peptide. This could possibly reduce the peptide loading level. If the multiple links occur through maleimides on different carrier proteins, the possibility of cross-linking of the carrier protein subunits through the peptide can result. Thiolation of OMPC primary amines with N-acetylcysteine lactone can achieve high levels of thiol groups which under appropriate buffer reaction conditions results in minimal cross-linking (via disulfide bond formation) of the carrier subunits (Marburg et al., 1986 J. Am. Chem. Soc. 108:5282-5287). Activation of the peptide with a single terminal electrophilic group (maleimide or alkyl halide) can lead to high levels peptide loading with a highly directed peptide to carrier coupling.

Linkers

A covalent linker joining a peptide to a carrier is stable under physiological conditions. Examples of such linkers are nonspecific cross-linking agents, monogeneric spacers and bigeneric spacers. Non-specific cross-linking agents and their use are well known in the art. Examples of such reagents and their use include reaction with glutaraldehyde; reaction with N-ethyl-N′-(3-dimethylaminopropyl) carbodiimide, with or without admixture of a succinylated carrier; periodate oxidation of glycosylated substituents followed by coupling to free amino groups of a protein carrier in the presence of sodium borohydride or sodium cyanoborohydride; periodate oxidation of non-acylated terminal serine and threonine residues can create terminal aldehydes which can then be reacted with amines or hydrazides creating Schiff base or hydrazones which can be reduced with cyanoborohydride to secondary amines; diazotization of aromatic amino groups followed by coupling on tyrosine side chain residues of the protein; reaction with isocyanates; or reaction of mixed anhydrides. See, generally, Briand, et al., 1985 J. Imm. Meth. 78:59.

Monogeneric spacers and their use are well known in the art. Monogeneric spacers are bifunctional and require functionalization of only one of the partners of the reaction pair before conjugation takes place. An example of a monogeneric spacer and its use involves coupling an immunogenic HCV peptide to one end of the bifunctional molecule adipic acid dihydrazide in the presence of carbodiimide. A diacylated hydrazine presumably forms with pendant glutamic or aspartic carboxyl groups of the carrier. Conjugation then is performed by a second coupling reaction with carrier protein in the presence of carbodiimide.

Bigeneric spacers and their use are well known in the art. Bigeneric spacers are formed after each partner of the reaction pair is functionalized. Conjugation occurs when each functionalized partner is reacted with its opposite partner to form a stable covalent bond or bonds. (See, for example, Marburg, et al., 1986 J. Am. Chem. Soc. 108:5282-5287; and Marburg, et al., U.S. Pat. No. 4,695,624.).

Peptide Coupling Load

An advantage of the present invention is that one can achieve various molar ratios of peptide to carrier protein in the conjugate. This “peptide coupling load” on carrier protein can be varied by altering aspects of the conjugation procedure in a trial and error manner to achieve a conjugate having the desired properties. For example, if a high coupling load is desired such that every reactive site on the carrier protein is conjugated to a peptide, one can assess the reactive sites on the carrier and include a large molar excess of peptide in the coupling reaction. If a low density coupling load is desired, one can include a molar ratio of less than 1 mol peptide per mole of reactive sites on the carrier protein.

The particular conditions one chooses will ultimately be guided by the yields achieved, physical properties of the conjugate, the potency of the resulting conjugate, the patient population and the desired dosage one wishes to administer. If the total protein in the vaccine is not an important consideration, one could formulate doses of conjugates of differing coupling loads and different immunogenicities to deliver the same effective dose. However, if total protein or volume is an important consideration, for example, if the conjugate is meant to be used in a combination vaccine, one may be mindful of the total volume or protein contributed by the conjugate to the final combination vaccine. One could then assess the immunogenicity of several conjugates having differing coupling loads and thereafter choose to use a conjugate with adequate immunogenicity and a level of total protein or volume acceptable to add to the combination vaccine.

Generally, there are two main obstacles for obtaining a high peptide load: (i) solubility of the ensuing conjugate, and (ii) solubility of the peptide. These properties are not independent, and manipulations, which improve the latter, can be detrimental to the former. Hence, it is often difficult to obtain a high peptide load.

Therefore, it can be desirable to modify the sequence of a peptide as described in U.S. Patent Application 60/530,867, filed Dec. 18, 2003. That application describes a method for increasing the immunogenicity of a peptide. The method comprises adjusting the isoelectric point (pI) of a peptide by modifying the peptide, and conjugating the peptide to a carrier. As used herein, “adjusting the pI of a peptide” means changing the pI of the peptide to such a range that both the peptide load and the solubility of the conjugate are increased. Frequently, the pI of the peptide is lowered to the range.

The pI of a peptide can be determined either with experiment such as Isoelectric focusing (IEF), or with calculation using appropriate software. As described in U.S. Patent Application 60/530,867, the pI, of the peptides can be modified in various ways which change the overall charge of the peptide. The modification can be any change or changes to the peptide that result in the change in the charges of the peptide. The modification can include the replacement, addition, or deletion of amino acid residues in the peptide. The modification can also include modification of the side chains of the residues or N-terminal amino group or C-terminal carboxylate group of the peptide. The methods of such modifications are within the knowledge of one skilled in the art.

The peptide should be modified outside of the immunogenically active sequence, i.e., the desired epitope, thus ensuring maintenance of the immunological properties. The modification should neither involve nor interfere with the desired epitope in the peptide. Since the modifications should not impact on the immunological properties of the peptide-conjugate, changes are preferably introduced at the N and/or C termini of the peptide.

One should also be mindful that the highest coupling load may not always yield the most immunogenic conjugate. Peptide length and coupling load on any given carrier protein may affect the overall immunogenicity of the conjugate. Therefore, one should assess the immunogenicity of a range of coupling loads of any particular peptide on any particular carrier protein. With that information one can then manufacture and formulate vaccines to provide appropriate dosages of conjugate to stimulate acceptable immunogenic responses in patients.

Formulations

The vaccine of the present invention can be formulated according to methods known and used in the art. Guidelines for pharmaceutical administration in general are provided in, for example, Modern Vaccinology, Ed. Kurstak, Plenum Med. Co. 1994; Remington's Pharmaceutical Sciences 18th Edition, Ed. Gennaro, Mack Publishing, 1990; and Modern Pharmaceutics 2nd Edition, Eds. Banker and Rhodes, Marcel Dekker, Inc., 1990.

Conjugates of the present invention can be prepared as acidic or basic salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

It is preferred that the adjuvant is chosen as appropriate for use with the particular carrier protein used in the conjugate as well as the ionic composition of the final formulation. Consideration should also be given to whether the conjugate alone will be formulated into a vaccine or whether the conjugate will be formulated into a combination vaccine. In the latter instance one should consider the buffers, adjuvants and other formulation components that will be present in the final combination vaccine.

Aluminum based adjuvants are commonly used in the art and include Aluminum phosphate, Aluminum hydroxide, Aluminum hydroxy-phosphate and aluminum hyrdoxy-sulfate-phosphate. Trade names of adjuvants in common use include ADJUPHOS, MERCK ALUM and ALHYDROGEL. The conjugate can be bound to or co-precipitated with the adjuvant as desired and as appropriate for the particular adjuvant used.

Non-aluminum adjuvants can also be used. Non-aluminum adjuvants include QS21, Lipid-A and derivatives or variants thereof, Freund's complete or incomplete adjuvant, neutral liposomes, liposomes containing vaccine and cytokines or chemokines.

It is preferred that the vaccine be formulated with an aluminum adjuvant. In other preferred embodiments, the vaccine is formulated with both an aluminum adjuvant and QS21.

It is preferable, in certain embodiments, to formulate the M2 peptide-protein conjugates with immunogens from influenza type B, like those described in the present application, and/or with immunogens from Haemophilus influenza, hepatitis viruses A, B, or C, human papilloma virus, measles, mumps, rubella, varicella, rotavirus, Streptococcus pneumonia and Staphylococus aureus. Additionally, the vaccine of the present invention can be combined with other antigenic components of influenza type A virus including, in particular, epitopes derived from hemaglutinin and neuraminidase. In this manner a combination vaccine can be made. Combination vaccines have the advantages of increased patient comfort and lower costs of administration due to the fewer inoculations required.

When formulating combination vaccines one should be mindful of the various buffers and adjuvants used with the other immunogens. Some buffers may be appropriate for some immunogen-adjuvant pairs and not appropriate for others. In particular, one should assess the effects of phosphate levels on the various immunogen-adjuvant pairs to assure compatibility in the final formulation.

Vaccination

The vaccine of the present invention can be administered to a patient by different routes such as intravenous, intraperitoneal, subcutaneous, or intramuscular. A preferred route is intramuscular. Suitable dosing regimens are preferably determined taking into account factors well known in the art including age, weight, sex and medical condition of the subject; the route of administration; the desired effect; and the particular conjugate employed (e.g., the peptide, the peptide loading on the carrier, etc.). The vaccine can be used in multi-dose vaccination formats. It is expected that a dose would consist of the range of 1 μg to 1.0 mg total protein. In an embodiment of the present invention the range is 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. An immunologically effective dose is one that stimulates the immune system of the patient to establish a level immunological memory sufficient to provide long term protection against disease caused by infection with influenza virus. The conjugate is preferably formulated with an adjuvant.

The timing of doses depend upon factors well known in the art. After the initial administration one or more booster doses may subsequently be administered to maintain antibody titers. An example of a dosing regime would be a dose on day 1, a second dose at 1 or 2 months, a third dose at either 4, 6 or 12 months, and additional booster doses at distant times as needed.

A patient or subject, as used herein, is an animal. Mammals and birds, particularly fowl, are suitable subjects for vaccination. Preferably, the patient is a human. A patient can be of any age at which the patient is able to respond to inoculation with the present vaccine by generating an immune response. The immune response so generated can be completely or partially protective against disease and debilitating symptoms caused by infection with influenza virus.

It should be noted that a vaccine of this invention having only M2 peptide will not prevent infection of cells of the patient. This is because the M2 epitopes in the peptides of the vaccine are present at very low copy numbers on the influenza virus when it enters the patient and begins an infection. These M2 epitopes are typically seen only on the surface of cells that have been infected by the virus. Therefore, the immune response generated by vaccination with the M2 peptide-protein conjugate based vaccine is directed against infected cells. Without wishing to be bound to a particular theory of effectiveness, it is believed that the patient's immune response reduces viral burst size, attenuates overall viral infection and thereby essentially limits the infection to the initially infected cells.

An advantage of the vaccine of the present invention is that the immune response is generated against conserved epitopes of the influenza virus. Thus, administration of this vaccine will avoid the necessity of annual vaccination to maintain protection of a patient against influenza infection.

The present M2 peptide-protein conjugate vaccine can be formulated with other vaccines to yield a combination vaccine as described above. One can then inoculate a patient with the combination vaccine to generate an immune response against the M2 epitopes as well as the other immunogens in the combination vaccines.

### EXAMPLE 1

Preparation of Peptides

Synthetic peptides representing portions of the M2 protein sequence and containing C-terminal or N-terminal reactive bromoacetyl or maleimide groups were produced by solid phase chemical synthesis methods commonly practiced in the art.

For example, the C-terminal bromoacetylated M2 15-mer, CT-BrAcM2-15 mer, Ac-Ser-Leu-Leu-Thr-Glu-Val-Glu-Thr-Pro-Ile-Arg-Asn-Glu-Trp-Gly-Aha-Lys (N^{ε}-BrAc)-NH₂.TFA salt (SEQ ID NO:13), was synthesized as a protected resin bound peptide on an APPLIED BIOSYSTEMS 430A peptide synthesizer (APPLIED BIOSYSTEMS, CITY STATE). Starting with 0.5 mmol p-methylbenzhydrylamine (MBHA) resin, the protocol used a 4 fold excess (2 mmol) of each N^{a}-Boc protected amino acid. Side-chain protection was Lys (Fmoc), Trp (Formyl), Glu (OcHex), Arg (Tos), Thr (Bzl). Coupling was achieved using DCC and HOBT activation in methyl-2-pyrrolidinone (NMP). Acetic acid was coupled for the introduction of the N terminal acetyl group. Removal of the Boc group was performed using 1:1 TFA in methylene chloride (MeCl₂) and the TFA salt neutralized with diisopropylethylamine.

Following assembly of the protected peptide resin the formyl group on the Trp residue and the Fmoc protection on the N^{ε}-Lys residue were removed by manual treatment with 25% piperidine in NMP for 10 min. After washing the resin with NMP and MeCl₂ the N^{ε} amino group on Lys was reacted with bromoacetic anhydride (1 g/20 ml Me Cl₂) for 1 hr or until a negative ninhydrin reaction was observed. Following washing with MeCl₂ the resin was dried to a constant weight (2.70 g).

The protected peptide resin (2.70 g) was treated with HF (30 ml) and anisole (3 ml) as scavenger, for 1 hr at 0° C. After evaporation of the HF and anisole the residue was washed well with ether, filtered and extracted with 25% acetic acid in H₂O (200 ml). The filtrate was lyophylized to yield 1.5 g of crude product.

Purification of the crude product was achieved by preparative HPLC, Buffer A=0.1% TFA—H₂O; B=0.1% TFA—CH₃CN. The crude product (0.75 g) was dissolved in a minimum volume of 20% acetic acid—H₂O (≈100 ml) and pumped onto a C-18 reverse phase HPLC radial compression column (WATERS, Milford, Mass., DELTA-PAK, 15 μm, 100 Å, 5×30 cm) which had been equilibrated in 90% A-10% B buffer.

Charging of the peptide was followed by 1 L of the 90% A-10% B buffer mixture. A step gradient (10% B to 40% B) (100 mL increments) was generated from 1 L each of successively increasing concentration (5%) of mobile phase. A flow rate of 80 mLumin was used to elute the product. Detection was performed by monitoring the UV absorbance at 214 nm. Homogeneous product fractions (>98% pure by analytical HPLC) were pooled and freeze-dried to provide 200 mg of the CT-BrAcM2-15 mer peptide. Identity was confirmed by amino acid analysis and mass spectral analysis.

Synthesis of other C-terminal bromoacetylated peptides can be performed analogously. For example, the C-terminal Bromoacetylated M2 23-mer peptide, CT BrAc-M2-23 mer, Ac-Ser-Leu-Leu-Thr-Glu-Val-Glu-Thr-Pro-Ile-Arg-Asn-Glu-Trp-Gly-Ser-Arg-Ser-Asn-Asp-Ser-Ser-Asp-Aha-Lys (NE-BrAc)-NH₂.TFA salt, (SEQ ID NO:39), was synthesized as a protected resin bound peptide on an APPLIED BIOSYSTEMS 430A peptide synthesizer (APPLIED BIOSYSTEMS, CITY STATE) as follows. Starting with 0.75 m mol p-methylbenzhydrylamine (MBHA) resin, a double coupling protocol used an excess (2 mmol) of each N^{α}-Boc protected amino acid. Side-chain protection was Ser (Bzl) Lys (Fmoc), Trp (Formyl), Glu (OcHex), Arg (Tos), Thr (Bzl), Asp (OcHex). Coupling was achieved using DCC and HOBT activation in methyl-2-pyrrolidinone (NMP). Acetic acid was coupled for the introduction of the N terminal acetyl group. Removal of the Boc group was performed using 1:1 TFA in methylene chloride (MeCl₂) and the TFA salt neutralized with diisopropylethylamine. Following assembly of the protected peptide resin the formyl group on Trp and the Fmoc protection on N^{ε}-Lys was removed by manual treatment with 25% piperidine in NMP for 10 min. After washing the resin with NMP and MeCl₂ the N^{ε} amino group on Lys was reacted with bromoacetic anhydride (1 g/20 ml Me Cl₂) for 10 min. or until a negative ninhydrin reaction was observed. Following washing with MeCl₂ the resin was dried to a constant weight.

One half of the protected peptide resin (1.83 g) was treated with HF (20 ml) and anisole (2 ml) as scavenger, for 1 hr at 0° C. After evaporation of the HF and anisole the residue was washed well with ether, filtered and extracted with 25% acetic acid in H₂O (200 ml). The filtrate was lyophylized to yield 1.1 g of crude product.

Purification of the crude product was achieved by preparative HPLC, Buffer A=0.1% TFA—H₂O; B=0.1% TFA—CH₃CN. The crude product (1.1 g) was dissolved in a minimum volume of 20% acetic acid—H₂O (≈100 ml) and pumped onto a C-18 reverse phase HPLC radial compression column (WATERS, DELTA-PAK, Milford, MA, 15 μm, 100 Å, 5×30 cm) which had been equilibrated in 90% A-10% B buffer. Charging of the peptide was followed by 1 L of the 90% A-10% B buffer mixture. A step gradient (10% B to 40% B) (100 mL increments) was generated from 1 L each of a successively increasing concentration (5%) of mobile phase. A flow rate of 80 mL/min was used to elute the product. Detection was performed by monitoring the UV absorbance at 214 nm. Homogeneous product fractions (>98% pure by analytical HPLC) were pooled and freeze-dried to provide 224 mg of product CT-BrAc-M2-23 mer. Identity was confirmed by amino acid analysis and mass spectral analysis.

Synthesis of malimidated peptides is illustrated as follows. Peptide Ac-Ser-Leu-Leu-Thr-Glu-Val-Glu-Thr-Pro-Ile-Arg-Asn-Glu-Trp-Gly-Aha-Lys (N^{ε}-4-maleimidobutyryl-NH₂.TFA salt (SEQ ID NO:14) was synthesized starting with 0.75 m mol p-methylbenzhydrylamine (MBHA) resin. The protected resin bound peptide was synthesized on an APPLIED BIOSYSTEMS 430A peptide synthesizer (APPLIED BIOSYSTEMS, CITY STATE). The protocol used a 4 fold excess (2 mmol) of each N^{α}-Boc protected amino acid. Side-chain protection was Lys (Fmoc), Trp (Formyl), Glu (OcHex), Arg (Tos), Thr (Bzl). Coupling was achieved using DCC and HOBT activation in methyl-2-pyrrolidinone (NMP). Acetic acid was coupled for the introduction of the N terminal acetyl group. Removal of the Boc group was performed using 1:1 TFA in methylene chloride (MeCl₂) and the TFA salt neutralized with diisopropylethylamine. Following assembly of the protected peptide resin the formyl group on Trp and the Fmoc protection on N^{ε}-Lys was removed by manual treatment with 25% piperidine in NMP for 10 min. After washing the resin with NMP and MeCl₂ a 25% portion of the resin was removed (0.188 mmol) and the NE amino group on Lys was reacted with 4-maleimidobutyric acid (2 mmol) and 2 mmol of DCC and HOBT in NMP for 3 hrs or until a negative ninhydrin reaction was observed. Following washing with NMP and MeCl₂ the resin was dried to a constant weight (0.7 g).

The protected peptide resin (0.70 g) was treated with HF (15 ml) and anisole (1.5 ml) as scavenger, for 1 hr at 0° C. After evaporation of the HF and anisole the residue was washed well with ether, filtered and extracted with 25% acetic acid in H₂O (100 ml). The filtrate was lyophilized to yield 0.40 g of crude product.

Purification of the crude product was achieved by preparative HPLC, Buffer A=0.1% TFA—H₂O; B=0.1% TFA—CH₃CN. The crude product (0.40 g) was dissolved in a minimum volume of 20% acetic acid—H₂O (≈100 ml) and pumped onto a C-18 reverse phase HPLC radial compression column (DELTA-PAK, 15 μm, 100 Å, 5×30 cm, WATERS, Milford, MA) which had been equilibrated in 90% A-10% B buffer. Charging of the peptide was followed by 1 L of the 90% A-10% B buffer mixture. A step gradient (10% B to 35% B) (100 mL increments) was generated from 1 L each of a successively increasing concentration (5%) of mobile phase. A flow rate of 80 mL/min was used to elute the product. Detection was performed by monitoring the UV absorbance at 214 nm. Homogeneous product fractions (>98% pure by analytical HPLC) were pooled and freeze-dried to provide 94 mg of product. Identity was confirmed by amino acid analysis and mass spectral analysis.

Analytical HPLC Conditions

| Column: | Vydac 15 cm #218TP5415, C18. |
|---|---|
| Eluant: | Gradient 95:5 (0.1% TFA/Acetonitrile) to 5:95 |
| | (0.1% TFA/Acetonitrile) over 45 min. |
| Flow: | 1.5 ml/min. |
| Wavelength: | 214 nM, 254 nM. |
| Retention time: | 16.9 min. |
| Molecular formula: | C₉₉H₁₅₅N₂₅O₃₁ |
| Molecular weight: | 2190.13. |

Synthesis of a second maleimidated peptide, Ac-Ser-Leu-Leu-Thr-Glu-Val-Glu-Thr-Pro-Ile-Arg-Asn-Glu-Trp-Gly-Ser-Arg-Ser-Asn-Asp-Ser-Ser-Asp-Aha-Lys (N^{ε}-4-maleimidobutyryl)-NH₂.TFA salt (SEQ ID NO:23) is illustrated as follows. Starting with 0.50 m mol p-methylbenzhydrylamine (MBHA) resin, the protected resin bound peptide was synthesized on an APPLIED BIOSYSTEMS 430A peptide synthesizer (APPLIED BIOSYSTEMS, CITY STATE). A double coupling protocol used an excess (2 mmol) of each N^{ε}-Boc protected amino acid. Side-chain protection was Ser (Bzl) Lys (Fmoc), Trp (Formyl), Glu (OcHex), Arg (Tos), Thr (Bzl), Asp (OcHex). Coupling was achieved using DCC and HOBT activation in methyl-2-pyrrolidinone (NMP). Acetic acid was coupled for the introduction of the N terminal acetyl group. Removal of the Boc group was performed using 1:1 TFA in methylene chloride (MeCl₂) and the TFA salt neutralized with diisopropylethylamine. Following assembly of the protected peptide resin the formyl group on Trp and the Fmoc protection on N^{ε}-Lys was removed by manual treatment with 25% piperidine in NMP for 10 min. After washing the resin with NMP and MeCl₂ a 50% portion of the resin (0.25 mmol) was reacted with 4-maleimidobutyric acid (2 mmol) and 2 mmol of DCC and HOBT for 3 hrs or until a negative ninhydrin reaction was observed. Following washing with NMP and MeCl₂ the resin was dried to a constant weight (2.0 g).

The protected peptide resin (2.0 g) was treated with HF (20 ml) and anisole (2 ml) as scavenger, for 1.5 hrs at 0° C. After evaporation of the HF and anisole the residue was washed well with ether, filtered and extracted with 50% acetic acid in H₂O (200 ml). The filtrate was lyophilized to yield 1.0 g of crude product.

Purification of the crude product was achieved by preparative HPLC, Buffer A=0.1% TFA—H₂O; B=0.1% TFA—CH₃CN. The crude product (1.0 g) was dissolved in a minimum volume of 10% acetic acid—H₂O (100 ml) and pumped onto a C-18 reverse phase HPLC radial compression column (DELTA-PAK, 15 μm, 100 Å, 5×30 cm, WATERS, Milford, Mass.) which had been equilibrated in 85% A-15% B buffer. Charging of the peptide was followed by a gradient elution of 15% B to 45% B over 90 min. A flow rate of 80 mL/min was used to elute the product. Detection was performed by monitoring the UV absorbance at 214 nm. Homogeneous product fractions (>98% pure by analytical HPLC) were pooled and freeze-dried to provide 320 mg of product. Identity was confirmed by amino acid analysis and mass spectral analysis.

Analytical HPLC Conditions

| Column: | Vydac 15 cm #218TP5415, C18 |
|---|---|
| Eluant: | Gradient 95:5 (0.1% TFA/Acetonitrile) to 5:95 |
| | (0.1% TFA/Acetonitrile) over 45 min. |
| Flow: | 1.5 ml/min. |
| Wavelength: | 214 nM, 254 nM |
| Retention time: | 16.4 min |
| Molecular formula: | C₁₂₉H₂₀₃N₃₇O₄₈ |
| Molecular weight: | 3038.46 |

Thiol equivalents of the synthetic peptides were assayed. For example, NT-BrAcM2-15 (N-terminal bromoacetylated M2 15-mer SEQ ID NO: 11) and CT-BrAcM2-15 (C-terminal bromoacetylated M2 15-mer SEQ ID NO: 13) were dissolved in N₂-sparged 25 mM Borate, 0.15 M NaCl, 2 mM EDTA, pH 8.5 buffer at a final concentration of 7.5 mg peptide powder/mL. The pH was adjusted to 8.5 with 0.97 N NaOH. The solution was 0.2 micron filtered. An aliquot was assayed for BrAcetyl equivalents by a thiol consumption assay as follows. N-acetyl-cysteine dissolved in N₂-sparged 25 mM borate, 0.15 M NaCl, 2 mM EDTA, pH 8.5 buffer was added (50 μM final concentration) to an appropriate dilution of peptide (˜15-30 μM final concentration) and to an equal volume of buffer and incubated for 30 min at room temperature. After the incubation, 5,5′-dithio-bis-[2-nitrobenzoic acid] (DTNB; Ellman's reagent) is added (5 mM final concentration using a 50 mM DTNB stock in N₂ saturated 0.1M Na phosphate, 0.1 M NaCl, 2 mM EDTA, pH 7). After incubation for 15 min at room temperature the thiol concentration was determined using ε412 nm, 1 cm=14.15×10³ M⁻¹ cm⁻¹ after subtracting the appropriate DTNB blank. The difference in free thiol in the presence and absence of the peptide estimates the thiol reactive equivalents.

Similarly, NT-MalM2-15 (N-terminal maleimidated M2 15-mer SEQ ID NO: 12) and CT-MalM2-15 (C-terminal maleimidated M2 15-mer SEQ ID NO: 14 were dissolved in N₂-sparged 0.1 M HEPES, 0.15 M NaCl, 2 mM EDTA, pH 7.3 buffer at a final concentration of 7.5 mg peptide powder/mL. The pH was adjusted to 7.3 with 0.97 N NaOH. The solution was 0.2 micron filtered. An aliquot was assayed for maleimide equivalents by a thiol consumption assay as follows. N-acetyl-cysteine dissolved in N₂-sparged 20 mM HEPES, 0.15 M NaCl, 2 mM EDTA, pH 7.3 buffer was added (50 μM final concentration) to an appropriate dilution of peptide (˜15-30 μM final concentration) and to an equal volume of buffer and incubated for 30 min at room temperature. After the incubation, DTNB is added (5 mM final concentration using a 50 mM DTNB stock in 0.1M Na phosphate, 0.1 M NaCl, 2 mM EDTA, pH 7). After incubation for 15 min at room temperature the thiol concentration was determined using ε412 nm, 1 cm=14.15×10³ M⁻¹ cm⁻¹ after subtracting the appropriate DTNB blank. The difference in free thiol in the presence and absence of the peptide estimates the thiol reactive equivalents.

For thiol-containing peptides (e.g.: SEQ ID NOs:1, 2, 3, 4, 10, etc.) peptides were dissolved (2.5-7.5 mg/mL) in ice-cold N₂-saturated 0.1 M HEPES, 2 mM EDTA, 0.15 M NaCl, pH 7.3 buffer and 0.2 micron filtered. The thiol content was measured by diluting an appropriate volume of the peptide into N₂ saturated 0.1M Na phosphate, 0.1 M NaCl, 2 mM EDTA, pH 7 buffer. DTNB was added to a final concentration of 5 mM using a 50 mM DTNB stock in 0.1 Na phosphate, 0.1 M NaCl, 2 mM EDTA, pH 7 buffer. After incubation for 15 min at room temperature the thiol concentration was determined using ε412 nm, 1 cm=14.15×10³ M⁻¹ cm⁻¹ after subtracting the appropriate DTNB blank.

Thiol Reactive Equivalents of Filtered Bromoacetyl or Maleimidated Peptides

| | | | Thiol Reactive |
|---|---|---|---|
| | [Thiol Reactive | | Equivalents per |
| | Equivalents]^{a} | [Peptide]^{b} | Peptide^{c} |
| PEPTIDE SAMPLES | μmol/mL | μmol/mL | mol/mol |
| NT-BrAcM2-15 | 0.71 | 3.37 | 0.21 |
| In Borate Buffer | n = 1 | n = 1 | |
| OMPC-FLU-9- | | | |
| BrAc Peptide | | | |
| NT-MalM2-15 | 3.12 | 2.98 | 1.05 |
| In HEPES Buffer | n = 1 | n = 1 | |
| OMPC-FLU-9- | | | |
| Mal Peptide | | | |
| CT-BrAcM2-15 | 0.91 | 3.06 | 0.30 |
| In Borate Buffer | n = 1 | n = 1 | |
| OMPC-FLU-10- | | | |
| Peptide BrAC | | | |
| CT-MalM2-15 | 3.31 | 3.11 | 1.06 |
| In Borate Buffer | n = 1 | n = 1 | |
| OMPC-FLU-10- | | | |
| PeptideMal | | | |

^{a}Determined by thiol consumption assay
^{b}Determined by AAA mean of asp, glu, gly, val, ile, leu, & arg values
^{c}NOTE: The [Thiol Reactive Equivalents] for NT-BrAcM2-15 is likely underestimated by ˜3-5 fold due to the slower reactivity of the bromoacetyl group in the thiol consumption assay.

Thiol Content of Filtered M2 Peptides Containing Cysteines.

Thiol/Peptide mol/mol

| PEPTIDE | Expected^{a} | Experimental^{b} |
|---|---|---|
| SEQ ID NO: 1 | 3 | 3.0 |
| SEQ ID NO: 2 | 1 | 0.9 |
| SEQ ID NO: 10 | 1 | 1.0 |

^{a}Based on the sequence of peptide.
^{b}Thiol content based on the modified Ellman's assay. Peptide concentration is based on single tryptophan of M2 peptide (assumes ε278 nm, 1 cm = 5,550 M⁻¹cm⁻¹ and ε288 nm, 1 cm = 4,550 M⁻¹ cm⁻¹. The concentrations used is the mean determined at these two wavelengths.

### EXAMPLE 2

Preparation of the Thiolated Outer Membrane Protein Complex (OMPC) of Neisseria meningitidis.

OMPC was obtained using techniques well known in the art and described by Fu U.S. Pat. No. 5,494,808. Thiolation of OMPC with N-acetylhomocysteine lactone was prepared by the general method described by Marburg et al. 1986 using aseptic technique. Thiolated OMPC underwent final ressuspension in N₂ saturated 25 mM Borate, 0.15 M NaCl, 2 mM EDTA, pH 8.5 for NT-BrAcM2-15 and CT-BrAcM2-15 and in 20 mM HEPES, 0.15 M NaCl, 2 mM EDTA, pH 7.3 for reaction with NT-MalM2-15 and CT-MalM2-15. Thiol content was measured by making the appropriate dilution of thiolated into OMPC into N₂ saturated 0.1 M Naphosphate, 0.1 M NaCl, 2 mM EDTA, pH 7 buffer. DTNB was added to a final concentration of 5 mM using a 50 mM DTNB stock in N₂ saturated 0.1 M Na phosphate, 0.1 M NaCl, 2 mM EDTA, pH 7 buffer. After incubation for 15 min at room temperature the thiol concentration was determined using E412 nm, 1 cm=14.15×10³ M⁻¹ cm⁻¹, after subtracting the appropriate DTNB blank and OMPC blank (no DTNB).

Properties of Thiolated OMPC.

| | [Thiol]^{a} | [Protein]^{b} | Thiol/Protein |
|---|---|---|---|
| THIOLATED OMPC SAMPLES | μmol/mL | mg/mL | μmol/mg |
| Thiolated OMPC | | | |
| In BORATE | | | |
| OMPC-FLU-9-1 | 1.63 | 6.35 | 0.26 |
| OMPC-FLU-10-1 | 1.54 | 6.09 | 0.25 |
| Thiolated OMPC | | | |
| In HEPES | | | |
| OMPC-FLU-9-2 | 1.72 | 6.57 | 0.26 |
| OMPC-FLU-10-2 | 1.55 | 6.29 | 0.25 |

^{a}Determined by modified Ellman's assay.
^{b}Determined by modified Lowry assay.

### EXAMPLE 3

Preparation of the Maleimidated or Alkylhalide-Activated OMPC.

All manipulations were carried out aseptically. Sterile OMPC in H₂O (5.5 mg/mL) was made 50 mM in NaHCO₃ pH 8.5±0.1 by addition of the appropriate volume of sterile 0.5 M NaHCO₃. Sulfosuccinimdyl 4-(N-maleimidomethyl) cyclohexane-1-carboxylate (sSMCC) or sulfosuccinimdyl (4-iodocetyl)aminobenzoate (sSIAB) (10 mM stock in ice-cold H₂O; chemicals from PIERCE CHEMICAL CO., ROCKFORD, Ill.) were added drop-wise to the buffered OMPC while gently mixing to give a final concentration of 2.5 mM sSLAB or sSMCC and an OMPC concentration of ˜3.8 mg/mL. Bromoacetic acid N-hydroxysulfosuccinimide ester can also be used. The reaction is aged for 1 h, in the dark at 4° C. After 1 h, the reaction mixture is adjusted to pH 7.3 with sterile 1 M Na phosphate and is exhaustively dialyzed in a 300 K molecular weight cut-off (MWCO) DISPODIALYZER® (SPECTRUM INDUSTRIES, INC., RANCO DOMINGUEZ, Calif.) against sterile 6.3 mM Na phosphate, pH 7.3, 0.15 M NaCl at 4° C. over a 12-24 h period. Alternatively, the pH-adjustment can be eliminated and the reaction mixture can be directly dialyzed. 20 mM HEPES, 0.15 M NaCl, 2 mM EDTA, pH 7.3, other appropriate buffers, or water can also be used for the dialysis. The SIAB dialysis was performed in the dark. N₂-sparging of the dialysis buffer can be added.

A preferred dialysis buffer for sSIAB activated OMPC mixture would be 50 mM in NaHCO₃ pH 8.5±0.1. The dialyzed activated OMPC is assayed for thiol reactive equivalents using a N-acetyl-cysteine consumption assay as described above for the peptides, except the assay buffer was 0.1M Na phosphate, 0.1 M NaCl, 2 mM EDTA, pH 7 and the N-acetylcysteine incubation period was 15 min. An OMPC blank (no DTNB) is run to correct for its contribution at 412 nm. Protein is measured by the modified Lowry. For the maleimide activation at pH 8.5 a level 0.09-0.12 micromoles of maleimide equivalents/mg of Lowry protein is typically achieved. This level is approximately 2-3 fold higher than the values obtained at pH 7.3. The activated OMPC is made 2 mM EDTA final concentration using a sterile 0.5 M EDTA, pH 8 stock.

### EXAMPLE 4

Conjugation of M2 peptide to Thiolated OMPC.

Thiolated OMPC was conjugated with M2 peptides NT-BrAcM2-15 (N-terminal bromoacetylated M2 15-mer SEQ ID NO: 11), CT-BrAcM2-15 (C-terminal bromoacetylated M2 15-mer SEQ ID NO: 13), NT-MaIM2-15 (N-terminal maleimidated M2 15-mer SEQ ID NO:12) and CT-MalM2-15 (C-terminal maleimidated M2 15-mer SEQ ID NO: 14) as follows using aseptic technique. Thiolated OMPC was added to different amounts of peptide and gently mixed. The reaction mixtures were aged without mixing at 4° C. overnight in the dark.

The reactions were then capped and desalted using aseptic technique. The NT-BrAcM2-15 and CT-BrAcM2-15/thiolated OMPC conjugation reactions were capped by making the reaction mixtures 5 mM in N-ethylmalimide (NEM) to react with excess thiols on the OMPC and aging for 4 h at 4° C. in the dark. The capped reaction mixture was desalted by dialysis in a 300 K MWCO DISPODIALYZER® against sterile 0.15 M NaCl at 4° C. The NT-MalM2-15/thiolated OMPC conjugation reactions were capped by making the reaction mixture 5 mM in iodoacetamide and aging overnight at 4° C. in the dark. The capped reaction mixture was desalted by dialysis in a 300 K MWCO DISPODIALYZER® against sterile 0.15 M NaCl at 4° C.

### EXAMPLE 5

Conjugation of M2 Peptide to Malimidated or Iodoacetylated OMPC.

Conjugation of maleimidated OMPC or iodoacetyled OMPC (alternatively bromoacetylated OMPC) with thiol-containing M2 peptides (SEQ ID NO: 1) was as follows using aseptic techniques. M2 peptide was added drop-wise to gently mixed maleimidated or iodoacetylated OMPC at a thiol/maleimide mol ratio of ˜3. The reverse addition, e.g., OMPC into peptide, can also be made, and is preferred. The reaction mixture is aged 12-24 h at 4° C. in the dark without mixing. Excess thiol reactive groups on OMPC were quenched (“capped”) with 0.2 micron filtered beta-mercaptomethanol (15 mM final concentration) by allowing the reagent to react with the conjugate for 3-4 h without mixing at 4° C. in the dark. The capped reaction was exhaustively dialyzed in a 300 K MWCO DISPODIALYZER® against sterile 0.15 M NaCl at 4° C.

### EXAMPLE 6

Analysis of the Conjugates

For the measurement OMPC protein or the measurement of protein plus peptide in the conjugates a modified Lowry assay was used. In this assay, protein samples were precipitated with trichloroacetic acid in the presence of the carrier sodium deoxycholate (Bensadoun and Weinstein 1976 Anal. Biochem. 70:241-250). Protein pellets were dissolved with SDS containing Lowry reagent A. BSA standard was treated in a like manner.

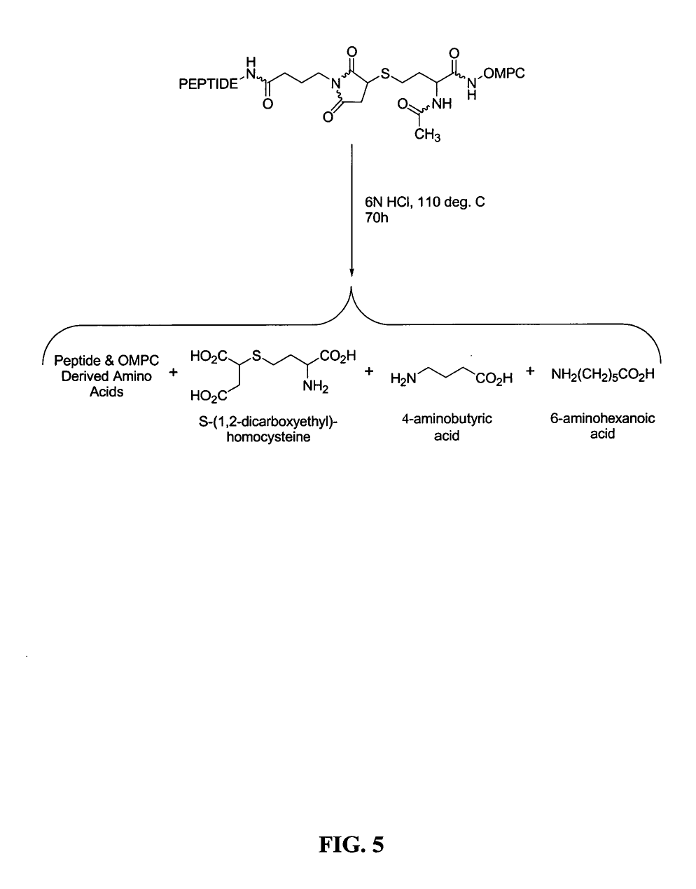

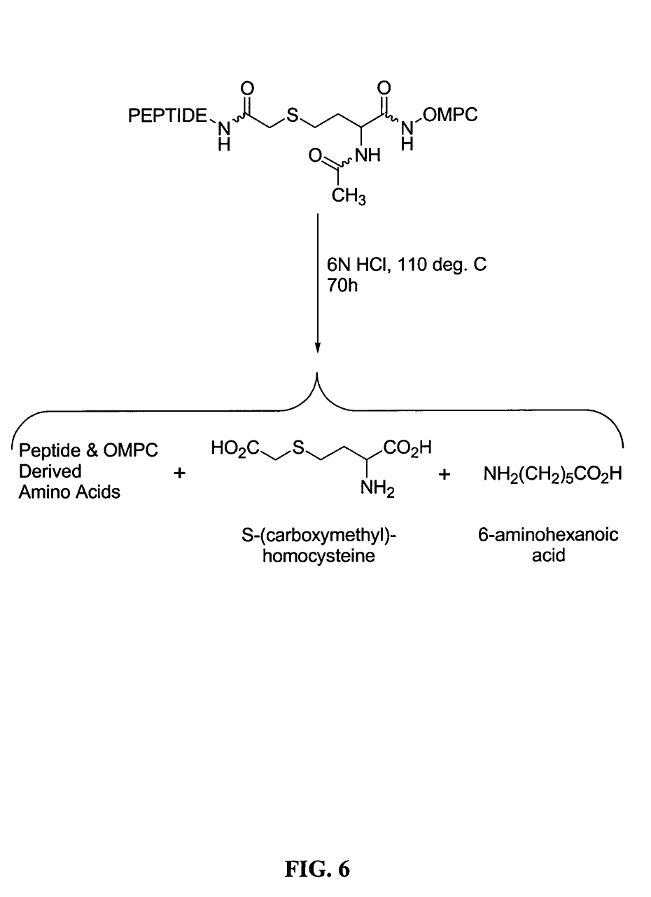

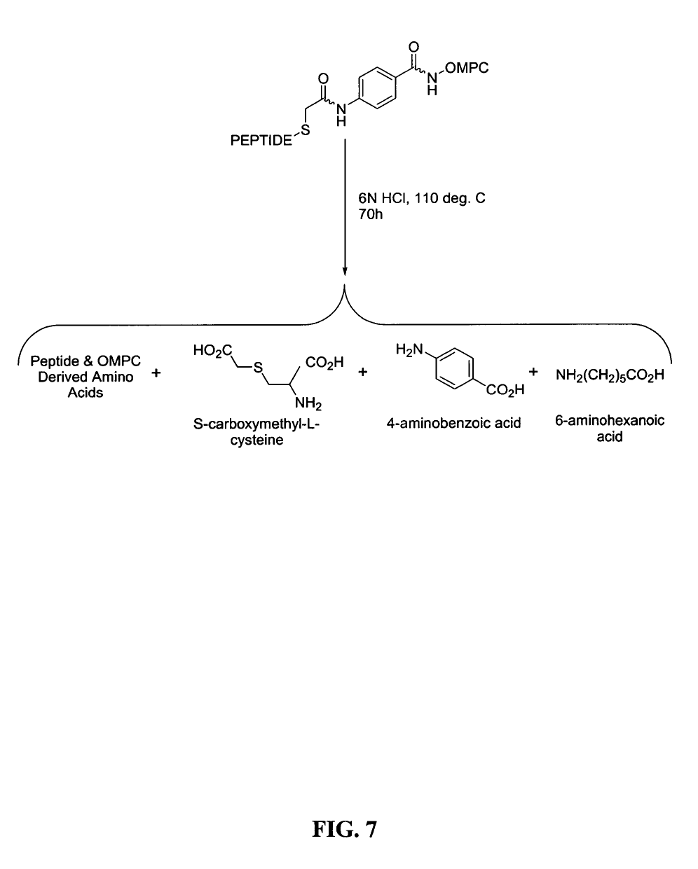

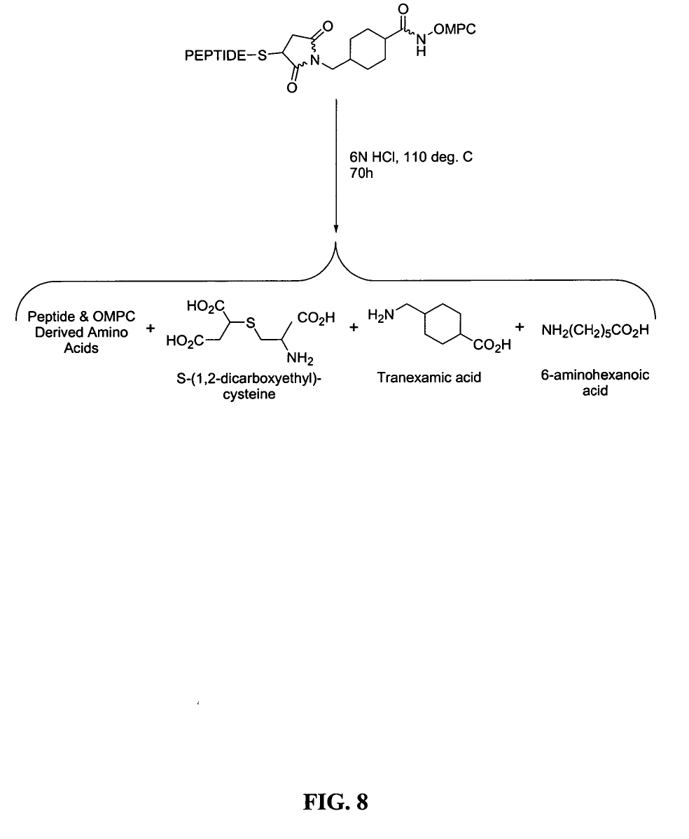

For amino acid analysis (AAA) samples were spiked with the internal standard, norleucine and hydrolyzed with 6 N HCl, 0.2% phenol (w/v) at 110° C. under vacuum for 70 h. See schemes V-VIII, FIGS. 5-8, for the expected amino acid hydrolysis products. After hydrolysis, samples were dried and resuspended in sample buffer and analyzed by cation exchange chromatography with post-column ninhydrin detection (BECKMAN Model 6300, Palo Alto, Calif.). The amino acid analysis can also be performed using other systems including ACCUTAG™ (WATERS CORP., MILFORD, Mass.) or AMINO ACID DIRECT (DIONEX CORP., SUNNYVALE, Calif.) which may provide advantages of sensitivity and/or resolution.

Peptide loading of the conjugate can be determined from the amino acid data by a least two methods. From a unique amino acid in the peptide (e.g., 6-aminohexanoic acid, AHA) the amount of peptide can be estimated. The amount of OMPC protein can be estimated from the amount of an amino present in OMPC but absent from the peptide. The Lowry protein number obtains a contribution from the peptide and at high peptide loadings can make an important contribution to the value obtained. An alternative method involves the use of a multiple regression, least squares analysis of the AAA data in a spread sheet format (Shuler et al. 1992 J. Immunol. Meth. 156:137-149). In general, the two methods generate values which agree within 20% of each other.

SDS-PAGE/staining analysis of reduced conjugate samples can provide qualitative evidence for peptide conjugation. For maleimide or iodoacetyl-activated OMPC/thiol containing M2 peptides conjugates analysis of quenched/activated OMPC can provide evidence for side reactions of SMCC or SIAB leading to cross-linking of the major class 2 protein of OMPC which exist as a trimer.

### EXAMPLE 7

Properties of the Thiolated OMPC/Maleimidated or Bromoacetyl M2 Conjugates.

Properties of Dialyzed NT-BrAcM2-15/Thiolated OMPC Conjugates.

| | Mod. Lowry^{a} | AAA^{b} | | |
|---|---|---|---|---|
| | “Protein + | Protein | | |
| REACTION SAMPLE | Peptide” | mg/mL | S-Carboxymethyl- | Peptide/OMPC^{d} |
| OMPC-FLU-9-1 | mg/mL | (Lowry/AAA) | homocysteine^{c} | mol/mol |
| A | 1.22 | 0.82 (1.49) | Yes | 5,122 |
| 2.8 μmol OMPC thiol + | | | | |
| 5.7 μmol peptide | | | | |
| B | 1.78 | 1.54 (1.16) | Yes | 3,662 |
| 2.8 μmol OMPC thiol + | | | | |
| 2.9 μmol peptide | | | | |
| C | 2.29 | 1.95 (1.17) | Yes | 3,258 |
| 2.8 μmol OMPC thiol + | | | | |
| 1.4 μmol peptide | | | | |
| D | 2.17 | 2.05 (1.06) | Yes | 2,398 |
| 2.8 μmol OMPC thiol + | | | | |
| 0.7 μmol peptide | | | | |
| E | 1.64 | 1.64 (1.00) | No | NA |
| 2.8 μmol OMPC thiol + | | | | |
| no peptide | | | | |

^{a}Modified Lowry assay.
^{b}Based on the mean of the values calculated from AAA data assuming 0.42 μmol Lysine/mg Lowry protein and 0.63 μmol alanine/mg Lowry protein.
^{c}S-Carboxymethylcysteine analysis was qualitative.
^{d}Based on the protein value determined by AAA, an assumed OMPC MW = 40 × 10⁶ and the AAA protein/6-aminohexanoic acid (Aha) value to give moles of peptide.

Properties of Dialyzed NT-MalM2-1 5/Thiolated OMPC Conjugates.

| | Mod. Lowry^{a} | AAA^{b} | | |
|---|---|---|---|---|
| | “Protein + | Protein | | |
| REACTION SAMPLE | Peptide” | mg/mL | S-Dicarboxyethyl- | Peptide/OMPC^{d} |
| OMPC-FLU-9-2 | mg/mL | (Lowry/AAA) | homocysteine^{c} | mol/mol |
| A | 2.91 | 2.40 (1.21) | Yes | 4,300 |
| 2.9 μmol OMPC thiol + | | | | |
| 2.8 μmol peptide | | | | |
| B | 2.53 | 2.29 (1.10) | Yes | 3,872 |
| 2.9 μmol OMPC thiol + | | | | |
| 1.4 μmol peptide | | | | |
| C | 2.21 | 2.07 (1.07) | Yes | 2,606 |
| 2.9 μmol OMPC thiol + | | | | |
| 0.7 μmol peptide | | | | |
| D | 0.65 | 0.59 (1.10) | No | NA |
| 2.9 μmol OMPC thiol + | | | | |
| no peptide | | | | |

^{a}Modified Lowry assay.
^{b}Based on the mean of the values calculated from AAA data assuming 0.42 μmol Lysine/mg Lowry protein and 0.63 μmol alanine/mg Lowry protein.
^{c}S-Dicarboxyethylhomocysteine analysis was qualitative.
^{d}Based on the protein value determined by AAA, an assumed OMPC MW = 40 × 10⁶ and the AAA protein/6-minohexanoic acid (Aha) value to give moles of peptide.

Properties of Dialyzed CT-BrAcM2-15/Thiolated OMPC Conjugates.

| | Mod. Lowry^{a} | AAA^{b} | | |
|---|---|---|---|---|
| | “Protein + | Protein | | |
| REACTION SAMPLE | Peptide” | mg/mL | S-Carboxymethyl- | Peptide/OMPC^{d} |
| OMPC-FLU-10-1 | mg/mL | (Lowry/AAA) | homocysteine^{c} | mol/mol |
| A | 2.27 | 2.03 (1.11) | Yes | 4,783 |
| 2.6 μmol OMPC thiol + | | | | |
| 5.2 μmol peptide | | | | |
| B | 2.19 | 2.04 (1.07) | Yes | 3,255 |
| 2.6 μmol OMPC thiol + | | | | |
| 2.6 μmol peptide | | | | |
| D | 2.00 | 2.44 (0.89) | Yes | 1,929 |
| 2.6 μmol OMPC thiol + | | | | |
| 0.65 μmol peptide | | | | |
| E | 1.69 | 1.92 (0.88) | No | NA |
| 2.6 μmol OMPC thiol + | | | | |
| no peptide | | | | |

^{a}Modified Lowry assay.
^{b}Based on the value calculated from AAA data assuming 0.63 μmol alanine/mg Lowry protein.
^{c}S-Carboxymethylhomocysteine analysis was qualitative.
^{d}Based on the protein value determined by AAA, an assumed OMPC MW = 40 × 10⁶ and the AAA protein/6-aminohexanoic acid(Aha) value to give moles of peptide.

Properties of Dialyzed CT-MalM2-15/Thiolated OMPC Conjugates.

| | Mod. Lowry^{a} | AAA^{b} | | |
|---|---|---|---|---|
| | “Protein + | Protein | | |
| REACTION SAMPLE | Peptide” | mg/mL | S-Dicarboxyethyl- | Peptide/OMPC^{d} |
| OMPC-FLU-10-2 | mg/mL | (Lowry/AAA) | homocysteine^{c} | Mol/mol |
| A | 2.72 | 2.45 (1.11) | Yes | 5,677 |
| 2.6 μmol OMPC thiol + | | | | |
| 2.5 μmol peptide | | | | |
| B | 2.51 | 2.64 (0.95) | Yes | 3,439 |
| 2.6 μmol OMPC thiol + | | | | |
| 1.3 μmol peptide | | | | |
| C | 2.43 | 2.47 (0.98) | Yes | 2,298 |
| 2.6 μmol OMPC thiol + | | | | |
| 0.65 μmol peptide | | | | |
| D | 2.14 | 2.38 (0.90) | Yes | 1,882 |
| 2.6 μmol OMPC thiol + | | | | |
| 0.33 μmol peptide | | | | |
| E | 1.90 | 1.98 (0.96) | No | NA |
| 2.6 μmol OMPC thiol + | | | | |
| no peptide | | | | |

^{a}Modified Lowry assay.
^{b}Based on the value calculated from AAA data 0.63 μmol alanine/mg Lowry protein.
^{c}S-Dicarboxyethylhomocysteine analysis was qualitative.
^{d}Based on the protein value determined by AAA, an assumed OMPC MW = 40 × 10⁶ and the AAA protein/6-aminohexanoic (Aha) value to give moles of peptide.

Generally, at an equal (mol) charge of peptide, the maleimidated peptide produced higher loading of peptide in the conjugate than the bromoacetylated peptide. The lower thiol kinetic reactivity of the bromoacetyl group compared to the maleimide group may be responsible for the difference.

### EXAMPLE 8

Properties of the Maleimidated OMPC and Selected Cysteine-Containing Peptide Conjugates.

Properties of Dialyzed Cysteine Containing Peptide/Maleimidated OMPC Conjugates.

| | Mod. Lowry^{a} | | Peptide/ |
|---|---|---|---|
| | “Protein + Peptide” | DCEC/ | OMPC^{c} |
| Peptide Conjugate | mg/mL | AHA^{b} | mol/mol |
| OMPC-FLU-2-4 | 2.09 | 3.1 | 1,110 |
| SEQ ID NO: 1 | | | |
| OMPC-FLU-2-5 | 2.84 | 0.99 | 2,873 |
| SEQ ID NO: 2 | | | |
| OMPC-FLU-3-5 | 2.40 | ND | 3,398 |
| SEQ ID NO: 10 | | | |

^{a}Modified Lowry assay.
^{b}S-Dicarboxyethylcysteine (DCEC) and 6-aminohexanoic (AHA) quantitation was by AAA. DCEC response factor/ASP response factor = 1.285.
^{c}Based on the protein value determined by AAA assuming 0.63 μmol alanine/mg Lowry protein., an assumed OMPC MW = 40 × 10⁶ and the 6-aminohexanoic (AHA) value to give moles of peptide.

Higher DCEC/AHA levels for M2 peptides containing multiple cysteine residues (e.g., SEQ ID NO:1) versus peptides with single cysteines (e.g., SEQ ID NO:2) suggests multiple maleimide/cysteine links per single M2 peptide. This could result in lower peptide loading in the conjugate and perhaps effect the immunogenicity of the conjugate. Smaller peptides (e.g., SEQ ID NO:10) appear to give higher peptide loading at equal peptide charges to the reaction for single cysteine containing M2 peptide conjugates. This effect is could be due to steric restraints at the maleimide sites on OMPC and/or charge differences near the reactive cysteine on the peptide. The reaction of maleimide with intrinsic nucleophiles (see Brewer and Riehm 1967 Anal. Biochem. 18:248-255) in OMPC creating cross-links during the activation and the desalting step was suggested by SDS-PAGE for quenched/maleimide-activated OMPC. There was less apparent cross-linking with activations at lower pH. SIAB-activated OMPC showed minimal cross-linking. Some of the maleimide groups may also have been converted to maleamic acid by a ring opening reaction of the imide. The maleamic acid is deficient in thiol reactivity. In general, higher peptide loadings for conjugates prepared using maleimidated peptides and thiolated OMPC versus similar peptide reactions using single cysteine containing peptides and maleimide activated OMPC were observed. Higher levels of activation of the OMPC using thiolation (˜0.26 micromole thiol/mg of protein) versus maleimidation (0.09-0.12 micromole maleimide/mg of protein) may account for the observation.

### EXAMPLE 9

Conjugates for Animal Studies.

For animal studies, conjugates were prepared using a peptide/OMPC thiol charge ratio (mol/mol) of ˜1 except for NT-BrAcM2-15 which used a ratio of ˜2. The aseptically prepared conjugates in 0.15 M NaCl were transferred for formulation on an aluminum adjuvant (MERCK alum).

Properties of Conjugates Used in Animal Studies.

| | AAA^{a} | |
|---|---|---|
| | Protein | Peptide/OMPC^{b} |
| Conjugate Samples | mg/mL | mol/mol |
| CT-BrAcM2-15 | 6.29 | 3,771 |
| CT-BrAc(SRS)M2-23 | 6.04 | 2,762 |
| OMPC-Flu-10-1G | 2.18 | 4,453 |
| NT-BrAcM2-15 | | |
| Quenched/Thiolated | 6.13 | NA |
| OMPC | | |
| CT-CysM2-15 | 2.70 | 4,576 |

^{a}Based on the value calculated from AAA data assuming 0.63 μmol alanine/mg Lowry protein.
^{b}Based on the protein value determined by AAA, an assumed OMPC MW = 40 × 10⁶ and 6-aminohexanoic (AHA) value to give moles of peptide.

### EXAMPLE 10

Formulation of Vaccine

The following conjugates were used in Example 11. The numbering of the “groups” refers to the groups of vaccinated animals. The conjugates used in formulations are CT-M2-15mer-ma-OMPC (Further referred to as conjugate “A”) Used in groups 1 to 3. CT-BrAcM2-15mer-OMPC (Further referred to as conjugate “B”) Used in groups 4 to 6. NT-BrAcM2-15-mer-OMPC (Further referred to as conjugate “C”) Used in groups 7 to 9. CT-BrAcM2(SRS)-23-mer-OMPC (Further referred to as conjugate “D”) Used in groups 10 to 12. Activated/quenched OMPC (Further referred to as compound “E”) Used in group 13. The dilutions are based on protein concentration determinations of the stocks by the Lowry method and the peptide load by amino acid analysis.

Step 1. Dilute conjugates A to D with 1×saline to 0.1 mg/mL peptide concentration. Dilute compound E to 0.5 mg/mL protein concentration.

Step 2. Add each solution from step 1 to pre-stirred 2×alum (MERCK ALUM, Prod. #39943, MERCK & CO, West Point, Pa.) in a ratio 1:1 for a final 50 mcg/mL peptide in lxalum (for compound E the final protein concentration was 0.25 mg/mL protein in Ixalum).

Step 3. Mix on rotating wheel for 2 hours at room temperature.

Step 4. Dilute the conjugates with Ixalum to reach the target peptide concentration.

4.1 Dilute solutions from step 3 with Ixalum as follows: 1 part solution with 4 parts Ixalum (v/v).

4.2 Mix on rotating wheel for 1 h at room temperature.

4.3 Set apart necessary volume of solutions at step 4.2 for groups 3, 6, 9, 12, (receiving 1 mcg peptide) and group 13 (receiving 5 mcg activated/quenched OMPC).

4.4 Mix leftover of solutions from 4.2 with 1×alum as follows: 1 part solution with 9 parts 1×alum (v/v).

4.5 Mix on rotating wheel for 1 h at room temperature.

4.6 Set apart necessary volume of solutions at step 4.5 for groups 2, 5, 8, 11 receiving 0.1 mcg peptide.

4.7 Mix leftover of solutions from 4.5 with 1×alum as follows: 1 part solution with 9 parts 1×alum (v/v).

4.8 Mix on rotating wheel for 1 h at room temperature.

4.9 The solutions at step 4.8 represent formulations for groups 1, 4, 7, 10 receiving 0.01 mcg peptide.

Step 5. Dispense into vials.

All the sample manipulations were performed under sterile conditions.

### EXAMPLE 11

Administration of Vaccine to a Mammal

Immunogenicity and protection of M2 peptide conjugate vaccines in mouse challenge model.

Four different M2 peptides conjugates were evaluated for their ability to elicit M2 peptide specific antibody responses and to confer protection against lethal influenza virus challenge in mice. The test conjugates are shown in the following Table.

| | | |
|---|---|---|
| Trivial name | M2 peptide sequence | Conjugation chemistry |

| | | |
|---|---|---|
| CT BrAc-15mer-OMPC | SLLTEVETPIRNEWG | Bromoacetyl peptide coupled at C- |
| SEQ ID NO: 13 | | terminus to thiolated OMPC |
| CT BrAc-23mer(SRS)-OMPC | SLLTEVETPIRNEWGSRSNDSSD | Bromoacetyl peptide coupled at C- |
| SEQ ID NO: 39 | | terminus to thiolated OMPC |
| NT BrAc-15mer-OMPC | SLLTEVETPIRNEWG | Bromoacetyl peptide coupled at N- |
| SEQ ID NO: 11 | | terminus to thiolated OMPC |
| CT 15mer-ma-OMPC | SLLTEVETPIRNEWGC | Thiolated peptide coupled at C- |
| SEQ ID NO: 10 | | terminus to Maleimide activated |
| | | OMPC |

All conjugates were all formulated on MERCK ALUM as described in Example 10. Each group of animals, consisting of ten (10) Female Balb/c mice per group, were immunized intramuscularly with 100 μl of a conjugate and boosted once with the same conjugate 3 weeks later. Each conjugate was tested in animals at three different doses, i.e., 0.01 μg, 0.1 μg and 1 μg, on the basis of the peptide content. For example, formulated conjugate A of Example 10 was administered at 0.01 μg to group 1, 0.1 μg to group 2 and 1 μg to group 3, while formulated conjugate B was administered at 0.01 μg to group 4, 0.1 μg to group 5 and 1 μg to group 6, and so on.

The control animals were immunized by the same schedule with non-conjugated OMPC formulated in the MERCK ALUM. Blood samples were collected at week 2 (post dose 1) and week 6 (post dose 2). Four weeks after the boost immunization, animals were challenged intranasally with LD90 (a dose that causes 90% mortality) of a mouse adapted A/Hong Kong/68 reassortant (HA gene from A/HK/68 and M2 gene from A/PR/8/34)(H₂N₂) (herein referred to as “A/HK/68 reassortant”). After challenge mice were monitored for weight loss and mortality daily for a total of 20 days.

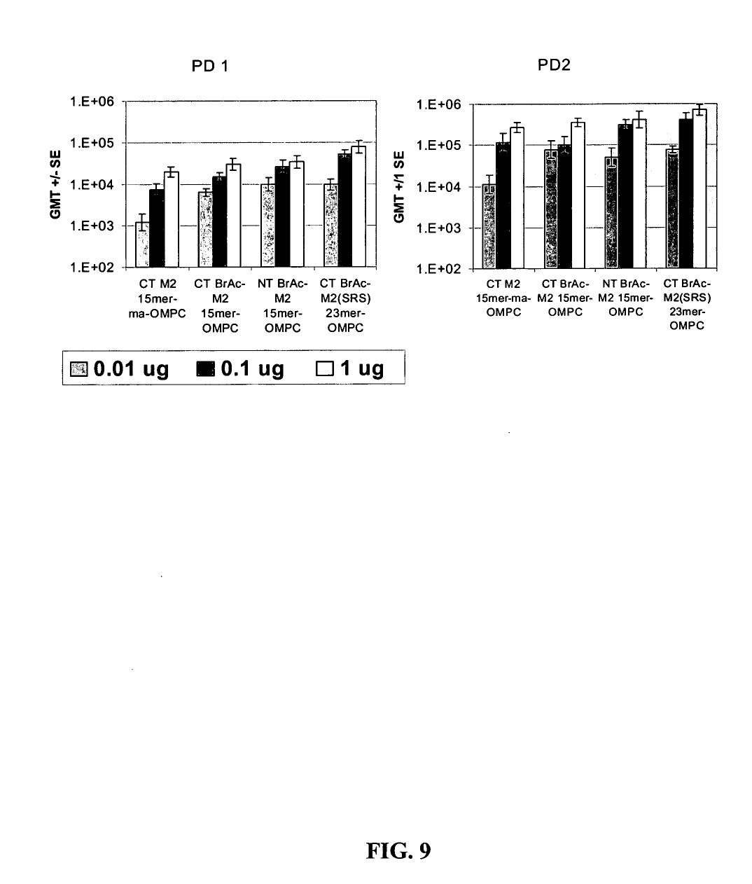

M2-specific antibody titers were determined by enzyme-linked immunosorbent assay (Elisa) using an unmodified 23 amino acid M2 peptide as the detection antigen. Both naive and OMPC control groups showed no detectable anti-M2 antibody titers. The results from the conjugate-vaccinated groups were shown in FIG. 9. Clear dose effects were observed at both PD1 and PD2 samples for all vaccine groups, indicating the vaccines were tested in a proper dose range. All conjugates were able to elicit significantly M2-specific antibody responses. After the boost immunization, the conjugates given at 1 ug dose all elicited specific antibody titers to half million or higher. Among the different vaccines, the CT BrAc 23mer(SRS)-OMPC elicited highest titers, whereas the CT 15mer-ma-OMPC had lowest titers. No apparent difference was observed between CT BrAc-15mer-OMPC and NT BrAc-15mer-OMPC, indicating that the peptide conjugated through N-terminus and that through the C-terminus have comparable immunogenicity.

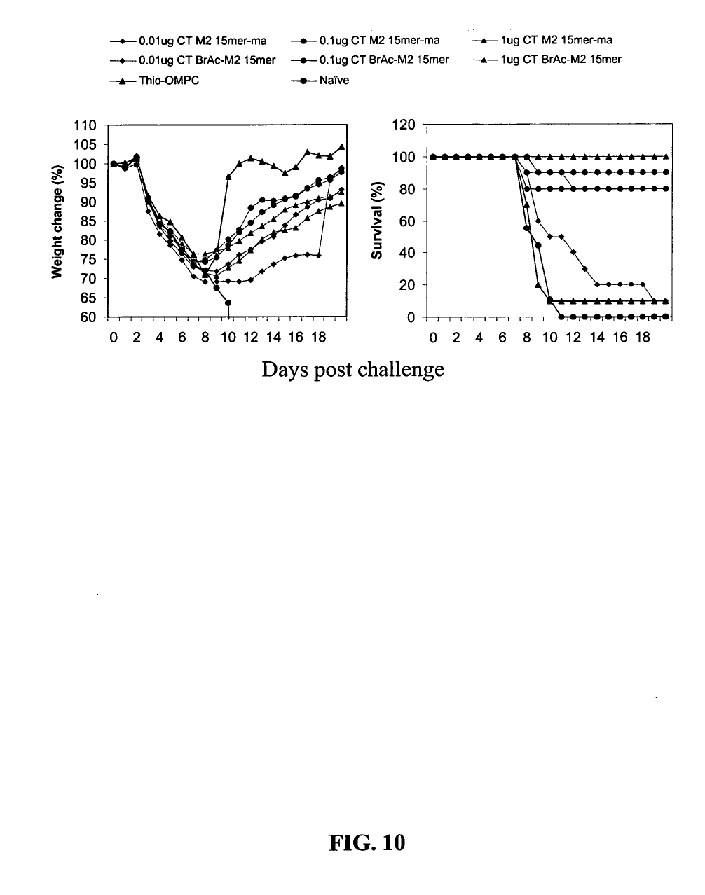

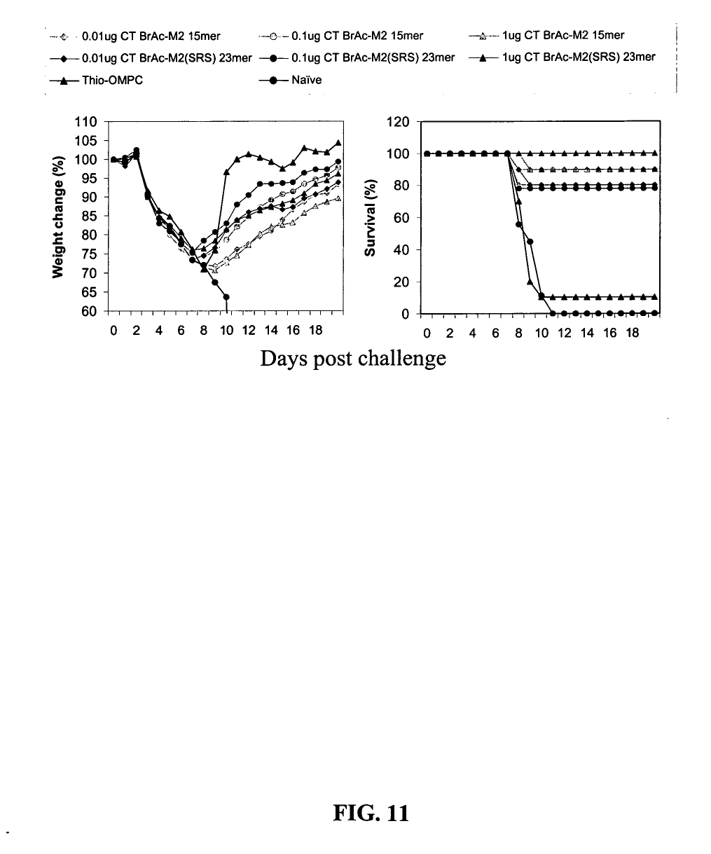

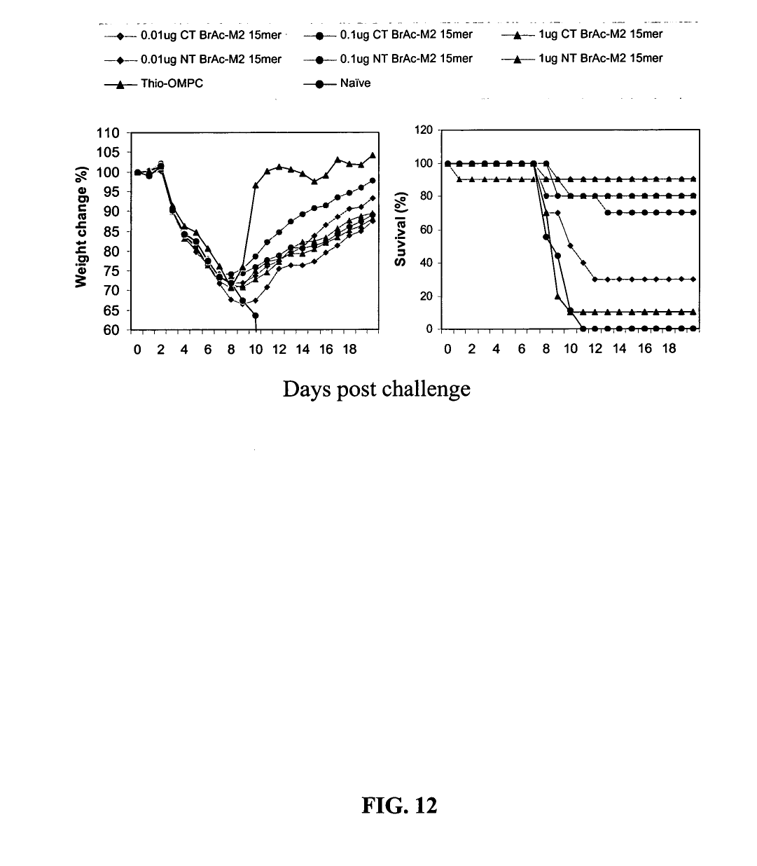

Following the lethal viral challenge, the control groups, as expected, showed 90 to 100% mortality. In contrast, all vaccine groups that received the 1 μg dose had 80 to 100% survival rate. This established that vaccines tested were able to confer protection against mortality. FIG. 10 shows the comparison between the CT BrAc-15mer-OMPC and CT 15-ma-OMPC. The most pronounced difference between the two conjugates is that at 0.01 ug dose the mice receiving CT BrAc-15mer-OMPC had 80% survival rate whereas the mice receiving CT 15-ma-OMPC had essentially the same mortality rate as the controls. This indicates that the CT BrAc-15mer-OMPC is more effective than CT 15-ma-OMPC with regard to protection against the lethal challenge. This contention is in fact consistent with the relative M2 antibody titers exhibited by these two groups. FIG. 11 shows the comparison between CT BrAc-15mer-OMPC and CT BrAc-23mer(SRS)—OMPC. In this case the difference between the two with respect to the mortality rate is not obvious. However, the groups receiving the CT BrAc 23mer(SRS)—OMPC showed overall less weight loss than did the groups receiving CT BrAc-15mer-OMPC, revealing a trend that the former could be potentially more protective. FIG. 12 shows the comparison between CT BrAc 15mer-OMPC and NT BrAc-15mer-OMPC. Overall, the groups receiving the CT BrAc-15mer conjugates showed higher survival rates than did the groups receiving the NT BrAc-15mer conjugates. In this experiment, all M2 peptide conjugates were protective against lethal viral challenge, and the M2 23mer(SRS) conjugated through the C-terminus to thiolated OMPC appears to be most effective vaccine.

### EXAMPLE 12

Peptide A/H3/HA₀-2

| SEQ ID NO: | Name | Peptide Sequence |
|---|---|---|
| 83 | A/H3/HA₀-2 | CGPEKQTRGLFGAIAGFIENG-NH₂ |

The peptide sequence of A/H3/HA₀-2 corresponds to intersubunit region spanning the cleavage site of the Hemagglutinin protein precursor HA₀ of Influenza A sequence, H3 subtype, Hong Kong A/68. In bold there are residues, such as a glycine and a cysteine residue at the N-terminus. These are required as spacer and as cysteinyl ligand to react with a maleimide activated OMPC carrier to generate the peptide-OMPC conjugate via thioether linkage. Peptide synthesis of A/H3/HA₀-2

The peptide was synthesized by solid phase using Fmoc/t-Bu chemistry on a Pioneer Peptide Synthesizer (APPLIED BIOSYSTEMS, Foster City, Calif.). The resin used was the Fmoc-Linker AM-Champion, 1% cross-linked (BIOSEARCH TECHNOLOGIES, INC., Novato, Calif.), a PEG-PS based resin derivatized with a modified Rink linker p-[(R,S)-α-[9H-Fluoren-9-yl-methoxyformamido]-2,4-dimethoxybenzyl]-phenoxyacetic acid (Rink, H. (1987) Tetrahedron Lett. 28, 3787-3789; Bernatowicz, M. S., Daniels, S. B. and Koster, H. (1989) Tetrahedron Lett. 30, 4645-4667).

All the acylation reactions were performed for 60 min with 4-fold excess of activated amino acid over the resin free amino groups. Amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropylethylamine). The side chain protecting groups were: tert-butyl for Asp, Glu, Ser, Thr and Tyr; trityl for Cys, Asn, His and Gln; tert-butoxy-carbonyl for Lys, Trp. At the end of the assembly, the dry peptide-resin was treated with 88% TFA, 5% phenol, 2% triisopropylsilane and 5% water (Sole, N. A., and Barany, G. (1992) J. Org. Chem., 57, 5399-5403) for 1.5 h at room temperature.

The resin was filtered and the solution was added to cold methyl-t-butyl ether in order to precipitate the peptide. After centrifugation the peptide pellets were washed with fresh cold methyl-t-butyl ether to remove the organic scavengers. The process was repeated twice. The final pellets were dried, resuspended in H₂O, 20% acetonitrile and lyophilized.

The crude peptide was purified by reverse-phase HPLC using a semi-preparative WATERS (MILFORD, MA) RCM DELTA-PAK™ C₋₁₈ cartridges (40×100 mm, 15 μm) using as eluents (A) 0.1% trifluoroacetic acid in water and (B) 0.1% trifluoroacetic acid in acetonitrile. We used the following gradient of B: 25%-40% over 20 min, flow rate 80 ml/min, with the peak corresponding to the product, eluting at a retention time (t_{R}) of 16′. Analytical HPLC was performed on a ULTRASHPERE, C₁₈ column, 25×4.6 mm, 5 μm with the following gradient of B: 20%-50% B in 20′, flow 1 ml/min. The purified peptide was characterized by electrospray mass spectrometry on a PERKIN-ELMER (WELLESLEY, Mass.) API-100: theoretical average mw is 2163.48 Da, found 2163.6 Da.

Conjugation of Peptide A/H3/HA₀-2 to OMPC

Various methods of purifying OMPC from the gram-negative bacteria have been devised (Frasch et al., J. Exp. Med. 140, 87 (1974); Frasch et al., J. Exp. Med. 147, 629 (1978); Zollinger et al., U.S. Pat. No. 4,707,543 (1987); Helting et al., Acta Path. Microbiol. Scand. Sect. C. 89, 69 (1981); Helting et al., U.S. Pat. No. 4,271,147). N. meningitidis B improved Outer Membrane Protein Complex (iOMPC) can be obtained using techniques well known in the art such as those described by Fu, U.S. Pat. No. 5,494,808.

To 2.9 mL of Neisseria meningitidis improved Outer Membrane Protein Complex (iOMPC) solution (6.84 mg/ml) was added 0.5 M NaHCO₃ (0.322 mL) to a final concentration of 50 mM, pH 8.5. To this was added drop-wise 0.83 mL of a 20 μM solution of the heterobifunctional crosslinker sulfosuccinimidyl 4-(N-maleimidomethyl)cyclohexane-1-carboxylate (sSMCC, PIERCE CHEMICAL CO., Rockford, Ill.) with a 2-fold excess (with respect to lysine residues of OMPC, 0.42 μmol lysine/mg OMPC protein). After aging the solution for 1 hour in the dark at 4° C., the pH was lowered to neutrality by adding a 1 M NaH₂PO₄ solution (46 μl). The solution was dialyzed at 4° C. using 300K MWCO DISPODIALYZER (SPECTRUM LABORATORIES INC., Rancho Dominguez Calif.) with 6-buffer changes (every 2 h) of 2 L, of 20 mM HEPES pH 7.3 (4-(2-Hydroxyethyl)piperazine-1-ethanesulfonic acid), 2 mM EDTA (Ethylenediaminetetracetic acid) to remove excess reagents. A total of 8.08 mL of activated OMPC (aOMPC) was recovered after dialysis.

A 0.7 mg/ml stock solution of the Cys-containing peptide ligand A/H3/HA₀-2, was prepared in degassed solution of 0.1 M HEPES, 2 mM EDTA pH 7.3. The thiol content of the peptide solution was determined by the Ellman assay (Ellman, G. L. (1959), Arch. Biochem. Biophys., 82, 70) and showed a —SH titre of 230 μM.

To define the maximum amount of peptide ligand that could be safely incorporated on aOMPC without causing precipitation, the conjugation reaction was first followed in small-scale trials where the aOMPC was incubated with increasing amounts of peptide ligand. The maximum number of maleimide groups that can be incorporated on the OMPC is limited by the total lysine residues on the OMPC, namely 0.42 μmoles lysine/mg OMPC. If one consider an average MW of 40×10⁶ Da for OMPC, this corresponds to 16,000 lysine moles/OMPCmol. Of these only a portion can be actually activated with sSMCC up to 35%, which corresponds to a maximum peptide load attainable of about 5000 moles. Therefore aOMPC was incubated with the following molar excesses of peptide ligand per OMPC mol: 500, 1000, 2000, 3000. After one hour, the samples were compared with an aOMPC sample to check for the presence of any precipitation or enhancement of turbidity.

In the case of A/H3/HA₀-2 the conjugation reaction gave a soluble product only when using a molar excess up to 2000 (of moles Cys-peptide/OMPC mol) for the 1 hour incubation reaction. Above that ratio, a complete precipitation of the OMPC solution occurred.

On the basis of these observations a large-scale reaction was performed: 4 mL (9.8 mg) of aOMPC were diluted with 2.08 mL of 20 mM HEPES, 2 mM EDTA pH 7.3. To this was added 2.08 ml of the peptide stock solution, drop-wise while gently vortexing, which corresponds to 2000 molar excess of peptide moles/OMPC mol. A sample of maleimide-activated OMPC solution was retained as blank for the determination of the peptide loading of the final conjugate. The conjugation reaction mixture was allowed to age for 17 h at 4° C. in the dark. Any residual maleimide groups on the OMPC were then quenched with β-mercaptoethanol to a final concentration of 15 mM (8.6 μL total volume added) for 1 h at 4° C. in the dark. The solution was dialyzed 4 times, 4 hour/change, with 1 L of 20 mM HEPES pH 7.3 at 4° C. with 300K MWCO DISPODIALYZER to remove unconjugated peptide and β-mercaptoethanol.

The concentration was determined by Lowry assay (Lowry, O. H., Rosebrough, N. J., Farr, A. L. and Randall, R. J. (1951), J. Biol. Chem., 193, 265), revealing 1.0 mg/mL for the OMPC-A/H3/HA₀-2. The conjugate and a aOMPC samples were hydrolyzed in evacuated, sealed glass tubes with azeotropic HCl for 70 hours at 110° C. The amino acid composition was determined by amino acid analysis. The conjugation load of peptide to OMPC protein was determined by comparing the conjugate amino acid composition with both that of the OMPC carrier and that of peptide ligand and by multiple regression, least squares analysis of the data (Shuler et al. Journal of Immunological Methods, 156, (1992) 137-149). For the conjugate OMPC and A/H3/HA₀-2, a molar ratio of peptide versus OMPC mole of 1160 was obtained.

### EXAMPLE 13

Peptide A/H3/HA₀-18

The pI of the peptide sequence of A/H3/HA₀-2 is 8.4 as calculated with ProMaC (Protein Mass Calculator) software v. 1.5.3. The sequence was engineered as to lower the value of pI of the peptide to 4.1, thus obtaining peptide HA₀-18, which share with A/H3/HA₀-2 the same sequence from the influenza HA₀ precursor. In bold are residues required for conjugation, spacing and pI engineering.

| | | |
|---|---|---|
| SEQ ID | | |
| NO: | Name | Peptide Sequence¹ |

| | | |
|---|---|---|
| 102 | A/H3/HA₀-18 | Ac-CGPEKQTRGLFGAIAGFIENGE-OH |

¹Ac—, acetyl, CH₃—CO—

Peptide Synthesis of A/H3/HA₀-18

The peptide was synthesized as described for A/H3/HA₀-2. To produce the peptide C-terminal acid, the peptides were synthesized on a Champion PEG-PS resin (BIOSEARCH TECHNOLOGIES, INC., Novato, Calif.) that had been previously derivatized with the 4-hydroxymethylphenoxyacetic acid linker using DIPCDI/HOBt as activators. The first amino acid, Glutamate, was activated as symmetrical anhydride with DIPC (diisopropylcarbodiimide) and esterified to the resin in the presence of a catalytic amount DMAP (dimethylaminopirydine). The acetylation reaction was performed at the end of the peptide assembly by reaction with a 10-fold excess of acetic anhydride in DMF.

The crude peptide HA₀-18 was purified by reverse-phase HPLC using a semi-preparative (WATERS, Milford, Mass.) RCM Delta-Pak™ C₁₈ cartridges (40×100 mm, 15 μm) using as eluents (A) 0.1% trifluoroacetic acid in water and (B) 0.1% trifluoroacetic acid in acetonitrile. We used the following gradient of B: 30%-45% over 20 min, flow rate 80 ml/min. Analytical HPLC was performed on a ULTRASHPERE, C₁₈ column (BECKMAN, FULLERTON, Calif.), 25×4.6 mm, 5 μm with the following gradient of B: 30%-45% B—in 20′-80% in 3′, flow 1 ml/min. The purified peptides were characterized by electrospray mass spectrometry on a PERKIN-ELMER (Wellesley, Mass.) API-100: theoretical average MW 2336.83 Da, found 2336 Da.

Conjugation of A/H3/HA₀-18 to OMPC

The iOMPC was activated as described in EXAMPLE 12 for A/H3/HA₀-2. A stock solution of the Cys-containing peptide ligand A/H3/HA₀-18, was prepared in degassed solution of 0.1 M HEPES, 2 mM EDTA pH 7.3. The thiol content of the peptide solutions was determined by the Ellman assay and showed a —SH titre of 200 μM. To define the maximum amount of peptide ligand that could be safely incorporated on aOMPC without causing precipitation, again the conjugation reaction was first followed in small-scale trials where the aOMPC was incubated with increasing amounts of peptide ligand. Namely aOMPC was incubated with the following molar excesses of peptide ligand per OMPC mol: 1000, 2000, 3000. After one hour, the samples were compared with a control aOMPC sample to check for presence of any precipitation or enhancement of turbidity. With the engineered sequence at lower pI, no precipitation or increase of turbidity was visible up to the highest molar excess of ligand used, 3000 moles/OMPC mol.

According to these observations, to 2 mL (4.6 mg) of aOMPC solution was added 1.68 mL of peptide stock solution (200 μM by Eliman assay, corresponding to a 3000 molar excess). The conjugation reaction mixture was allowed to age for 17 h at 4° C. in the dark. Any residual maleimide groups on the OMPC were then quenched with β-mercaptoethanol to a final concentration of 15 mM for 1 h at 4° C. in the dark. The solution was extensively dialyzed against 20 mM HEPES pH 7.3 at 4° C. with 300K MWCO DISPODIALYZER to remove unconjugated peptide and β-mercaptoethanol. The final conjugate was analyzed by Lowry assay and amino acid analysis as described for A/H³/HA₀-2. For the conjugate OMPC and A/H3/HA₀-18, a molar ratio of peptide versus OMPC mole of 2542 was obtained.

### EXAMPLE 14

Peptide A/H3/HA₀-17

| | | |
|---|---|---|
| SEQ ID | | |
| NO: | Name | Peptide Sequence |

| | | |
|---|---|---|
| 104 | A/H3/HA₀-17 | Suc-EPEKQTRGLFGAIAGFIENGC-OH |

¹Suc-, succinyl, HOOC—(CH₂)₂—CO—

The peptide sequence of A/H3/HA₀-17 corresponds to the cleavage site of the Hemagglutinin protein precursor HA₀ of Influenza A sequence, HK A/68, H3 subtype. The sequence is similar to that one of A/H3/HA₀-2 in EXAMPLE 1, but in this case the cysteine residue needed for conjugation with the maleimide activated carrier is at the C-terminus. The sequence was further modified to adjust the value of pI of the peptide to 4. The modifications include a Cys terminal carboxylate instead of amide, addition of a Glutamate and a succinyl at the N-terminus.

Peptide Synthesis of A/H3/HA₀-17

To produce the peptide C-terminal acid, the synthesis was performed on a Champion PEG-PS resin (Biosearch Technologies, Inc.) that had been previously derivatized with the 4-hydroxymethylphenoxyacetic acid linker using DIPCDI/HOBt as activators. The first amino acid, Glutamate, was activated as symmetrical anhydride with DIPC (diisopropylcarbodiimide) and esterified to the resin in the presence of a catalytic amount DMAP (dimethylaminopirydine). The assembly was performed as described for A/H3/HA₀-2. The succinylation reaction was performed at the end of the peptide assembly by reaction with a 10-fold excess of succinic anhydride in DMF.

The crude peptide A/H³/HA₀-17 was purified by reverse-phase HPLC using a semi-preparative WATERS (Milford, Mass.) RCM Delta-Pak™ C₁₈ cartridges (40×100 mm, 15 μm) using as eluents (A) 0.1% trifluoroacetic acid in water and (B) 0.1% trifluoroacetic acid in acetonitrile. We used the following gradient of B: 30%45% over 20 min, flow rate 80 ml/min. Analytical HPLC was performed on a ULTRASPHERE, C₁₈ column (BECKMAN, FULLERTON, Calif.), 25×4.6 mm, 5 μm with the following gradient of B: 30%-45%—in 20′-80% in 3′, flow 1 ml/min. The purified peptide was characterized by electrospray mass spectrometry on a PERKIN-ELMER (WELLESLEY, Mass.) API-100: theoretical average MW 2337.62 Da, found 2336,8 Da.

Conjugation of A/H3/HA₀-17 to aOMPC

The iOMPC was activated as described in EXAMPLE 12. A stock solution of HA₀-17, was prepared in degassed solution of 0.1 M HEPES, 2 mM EDTA pH 7.3. The thiol content of the peptide solutions was determined by the Ellman assay and showed a —SH titre of 200 μM. To define the maximum amount of peptide ligand that could be safely incorporated on aOMPC without causing precipitation, the conjugation reaction was first followed in small-scale trials where the aOMPC was incubated with increasing amounts of A/H3/HA0-17. Namely aOMPC was incubated with the following molar excesses of peptide ligand per OMPC mol: 1000, 2000, 3500. After one hour, the samples were compared with an aOMPC sample to check for the presence of any precipitation or enhancement of turbidity. With the engineered sequence at lower pI, no precipitation or increase of turbidity was visible up to the highest molar excess of ligand used, 3500 moles/OMPC mol.

On the basis of these observations, a large-scale reaction was performed on 3 mg (0.94 mL) of aOMPC. To this solution, 1.334 mL of the peptide stock solution were added drop-wise while gently vortexing, which corresponds to 3500 molar excess of peptide moles/OMPC mole. The conjugation reaction mixture was allowed to age for 17 h at 4° C. in the dark. Any unreacted maleimide groups on the OMPC were then reacted with β-mercaptoethanol to a final concentration of 15 mM for 1 h at 4° C. in the dark. The solution was extensively dialyzed against 20 mM HEPES pH 7.3 at 4° C. with 300K MWCO DISPODIALYZER (SPECTRUM LABORATORIES, INC., RANCHO DOMINGUEZ, Calif.) to remove unconjugated peptide and β-mercaptoethanol. The final conjugate was analyzed by Lowry assay and amino acid analysis as described for A/H3/HA₀-2. The analysis yielded a level of incorporation of 1860 moles of A/H3/HA₀-17 peptide/mol OMPC.

### EXAMPLE 15

PeptideA/H3/HA₂-25

| SEQ ID NO: | Name | Peptide Sequence |
|---|---|---|
| 77 | A/H3/HA₂-25 | GLFGAIAGFIENGWEGMVDGCE-OH |

The peptide sequence of A/H3/HA₂-25 corresponds to the fusion peptide region of the Hemagglutinin protein HA₂ of Influenza A sequence, H3 subtype, Hong Kong A/68. The sequence contains (in bold) a Cysteine for conjugation with maleimide activated OMPC, a Glycine residue as a spacer, and incorporation of a glutamate as C-terminal residue to adjust the pI to the value of 3.4.

Peptide Synthesis of A/H3/HA2-25

To produce the peptide C-terminal acid, the peptide was synthesized on a Champion PEG-PS resin (Biosearch Technologies, Inc.) that had been previously derivatized with the 4-hydroxymethylphenoxyacetic acid linker using DIPCDI/HOBt as activators. The first amino acid, Glutamate, was activated as symmetrical anhydride with DIPC (diisopropylcarbodiimide) and esterified to the resin in the presence of a catalytic amount DMAP (dimethylaminopirydine). The assembly was performed as described for A/H3/HA₀-2.

The crude peptide A/H3/HA₂-25 was purified by reverse-phase HPLC using a semi-preparative WATERS (Milford, Mass.) RCM Delta-Pak™ C₄ cartridges (40×100 mm, 15 μm) using as eluents (A) 0.1% trifluoroacetic acid in water and (B) 0.1% trifluoroacetic acid in acetonitrile. We used the following gradient of B: 40%-40%(5′)-60%(20′), flow rate 80 ml/min. Analytical HPLC was performed on a Phenomenex, Jupiter C₄ column, 15×4.6 mm, 5 μm with the following gradient of B: 35%-55%—in 20′-80% in 3′, flow 1 ml/min. The purified peptide was characterized by electrospray mass spectrometry on a PERKIN-ELMER (Wellesley, Mass.) API-100: theoretical average MW 2271,55 Da, found 2271,2 Da.

Conjugation of A/H3/HA₂-25 to aOMPC

The iOMPC was activated as described in EXAMPLE 12.

A solution of A/H3/HA₂-25, was prepared in degassed solution of 0.1 M HEPES, 2 mM EDTA pH 7.3. The thiol content of the peptide solutions was determined by the Ellman assay and showed a —SH titre of 250 μM.

To define the maximum amount of peptide ligand that could be safely incorporated on aOMPC without causing precipitation, the conjugation reaction was first followed in small-scale trials where the aOMPC was incubated with increasing amounts of A/H3/HA₂-25 Namely, aOMPC was incubated with the following molar excesses of peptide ligand per OMPC mol: 500, 1000, 2000, 4000, 6000. After one hour, the samples were compared with an aOMPC sample to check for the presence of any precipitation or enhancement of turbidity. With the engineered sequence at lower pI, no precipitation or increase of turbidity was visible up to the highest molar excess of ligand used, 6000 moles/OMPC mol.

According to these observations the large-scale reaction was performed on 6.3 mg (2.57 ml) of aOMPC. To this was added. 3.85 mL of the peptide stock solution drop-wise while gently vortexing which corresponds to 6000 molar excess of peptide moles/OMPC mole. The conjugation reaction mixture was allowed to age for 17 h at 4° C. in the dark. Any unreacted maleimide groups on the OMPC were then reacted with β-mercaptoethanol to a final concentration of 15 mM for 1 h at 4° C. in the dark. The solution was extensively dialyzed against 20 mM HEPES pH 7.3 at 4° C. with 300K MWCO DISPODIALYZER to remove unconjugated peptide and β-mercaptoethanol. The final conjugate was analyzed by Lowry assay and amino acid analysis as described for A/H3/HA₀-2. The analysis yielded a level of incorporation for A/H3/HA₂-25 of 2436 moles peptide/mol OMPC.

### EXAMPLE 16

Peptide B/HA₀-22

The peptide sequence of B/HA₀-22 corresponds to the cleavage site of the Hemagglutinin protein precursor HA₀ of Influenza B sequence, which is identical in influenza B viruses of the Victoria and Yamagata lineages, e.g. B/Ann Arbor/54, B/Hong Kong/330/2001, and B/Yamanashi/166/1998.

| SEQ ID NO: | Name | Peptide Sequence |
|---|---|---|
| 60 | B/HA₀-22 | BrAC-EGPAKLLKER↓GFFGAIAGFLEE-OH |

The sequence is modified with the introduction at the N-terminus of a bromoacetyl group to allow conjugation to thiolated OMPC (Tolman et al. Int. J. Peptide Protein Res. 41, 1993, 455-466; Conley et al. Vaccine 1994, 12, 445-451), of a Glycine spacer, and with modifications to adjust the pI value of the peptide. The modifications include a C-terminal carboxylate instead of carboxyamide, and addition of a Glutamate at the N- and C terminus Peptide synthesis of B/HA₀-22

The peptide was synthesized by solid phase using Fmoc/t-Bu chemistry on a Pioneer Peptide Synthesizer (Applied Biosystems, Foster City, Calif.). To produce the peptide C-terminal acid, the peptide was synthesized on a Champion PEG-PS resin (Biosearch Technologies, Inc., Novato, Calif.) that had been previously derivatized with the 4-hydroxymethylphenoxyacetic acid linker using DIPCDI/HOBt as activators. The first amino acid Glu was activated as symmetrical anhydride with DIPC (diisopropylcarbodiimide) and esterified to the resin in the presence of a catalytic amount DMAP (dimethylaminopirydine). The Bromoacetylation reaction was performed at the end of the peptide assembly by reaction with a 3-fold excess of bromoacetic acid using DIPCDI/HOBt as activators.

All the acylation reactions were performed for 60 min with 4-fold excess of activated amino acid over the resin free amino groups. Amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropylethylamine). The side chain protecting groups were: tert-butyl for Glu; tert-butoxy-carbonyl for Lys; 2,2,4,6,7-pentamethyldihydrobenzofuran-5-sulfonyl for Arg. At the end of the assembly, the dry peptide-resin was treated with 88% TFA, 5% phenol, 2% triisopropylsilane and 5% water (Sole, N. A., and Barany, G. (1992) J. Org. Chem., 57, 5399-5403) for 1.5 h at room temperature. The resin was filtered and the solution was added to cold methyl-t-butyl ether in order to precipitate the peptide. After centrifugation the peptide pellets were washed with fresh cold methyl-t-butyl ether to remove the organic scavengers. The process was repeated twice. The final pellets were dried, resuspended in H₂O, 20% acetonitrile and lyophilized.

The crude peptide was purified by reverse-phase HPLC using a semi-preparative WATERS (Milford, MA) RCM Delta-Pak™ C₋₁₈ cartridges (40×200 mm, 15 μm) using as eluents (A) 0.1% trifluoroacetic acid in water and (B) 0.1% trifluoroacetic acid in acetonitrile. We used the following gradient of B: 30%45% over 20 min, flow rate 80 ml/min. Analytical HPLC was performed on a ULTRASPHERE (BECKMAN, FULLERTON, Calif.), C₁₈ column, 25×4.6 mm, 5 μm with the following gradient of B: 30%-50% B in 20′,—80% in 3′, flow 1 ml/min. The purified peptide was characterized by electrospray mass-spectrometry on a Perkin-Elmer API-100: theoretical average mw is 2500.7 Da, found 2500.4 Da.

Conjugation of B/HA₀-22 to OMPC

The iOMPC starting material (150 mg) was first transferred into nitrogen-sparged, sterile filtered CM761 (0.11M Sodium Borate, pH 11.3) by ultracentrifugation (Ti-70 rotor, 50,000 RPM, 45 min, 4° C.), and dounce homogenization/resuspension at a concentration of 10 mg/mL. The protein was then thiolated using a solution of N-acetyl homocysteine thiolactone (NAHT) (0.89 g NAHT/g OMPC in nitrogen-sparged water) in conjunction with an EDTA-DTT solution (0.57 g EDTA/g OMPC, 0.11 g DTT/g OMPC, in CM761). The thiolation reaction was allowed to proceed for 4 hours at room temperature (˜20° C.). The thiolated iOMPC was then transferred into 25 mM sodium borate, pH 8.0 buffer via two ultracentrifugation (50,000 RPM, 45 min, 4° C.) and dounce homogenization/resuspension steps. At the end of thiolation, Lowry assay and Ellman's assay were performed before proceeding to the next step. The thiol content of the thiolated OMPC was 0.25 μmol thiol/mg.

65 mg of B/HA₀-22 was first dissolved in 25 mM sodium borate, pH 8.0 buffer at a concentration of 5 mg/mL. The pH of the peptide solution was then readjusted back to 8.0 with 1 N NaOH and then filtered with a 0.22 micron sterile filter. 53 mg of thiolated OMPC (at a mass charge ratio of 1.2 g peptide/g OMPC) was then added dropwise to the peptide stock solution with slight mixing. The conjugation reaction was allowed to proceed for 15.5 hours at room temperature without any agitation.

At the end of the conjugation reaction, the conjugate solution was transferred into six 300 kD MWCO DISPODIALYZERs, each with working volume of 5 mL. Three DISPODIALYZERs were put in a 4 L beaker with 3.5 to 4 L of sterile filtered water each. Gentle agitation was applied to each 4 L glass beaker containing both the conjugate as well as 3.5-4 L of sterile filtered water by using a 3-inch magnetic stirrer bar and adjustable speed stir plates. A total of 5 dialysis changes were carried out in sterile filtered water for a minimum of 6 hours per change to remove reaction by-products and excess free peptide.

The final conjugate was analyzed by Lowry assay and amino acid analysis as described for A/H3/HA₀-2. The analysis yielded a level of incorporation for B/HA₀-22 of 6500 moles peptide/mol OMPC.

### EXAMPLE 17

Mouse Challenge Experiment with Influenza Type A Virus in Mice Vaccinated with HA₀Peptide-OMPC Conjugates.

Female Balb/c mice were immunized intramuscularly with conjugates of HA peptides conjugated to OMPC. In the experiments using HA₀-21(H1) and HA₀-22(H3), the chemistry used for conjugation was thiolated OMPC and bromoacetylated peptide. In the. In the experiments using HA₀-25(H3L) and HA₀-25(H1), the chemistry used was maleimidyl-OMPC and cysteinyl-peptide. Conjugates were purified and prepared for formulation using standard procedures.

All the vaccines were formulated with Merck Alum or 20 ug of QS21 adjuvant and administered in a volume of 100 ul per mouse per injection. The mice were vaccinated at weeks 0, 2 and 4. The mice were challenged intranasally with a lethal dose of influenza virus PR8 or HK at week 7. Data are presented below.

Mouse Challenge Experiment with HA Peptide/OMPC Conjugate Vaccines

| | | Vaccine | | control | Challenge |
|---|---|---|---|---|---|
| Vaccine | Adjuvant | dose^{a} | survival | survival | Virus |
| A/H1/HA₀-21 | alum | 1 ug | 5/10 | 0/10 | PR8 (H1) |
| A/H3/HA₀-22 | alum | 1 ug | 1/10 | 1/10 | HK (H3) |
| A/H1/HA₀-25 | alum | 1 ug | 6/10 | 0/10 | PR8 (H1) |
| A/H3(L)/HA₀-25 | QS21 | 4 ug | 7/10 | 1/10 | HK (H3) |
| A/H3(L)/HA₀-25 | alum | 1 ug | 2/10 | 1/10 | HK (H3) |
| A/H3(L)/HA₀-25 | alum | 3 ug | 4/10 | 1/10 | HK (H3) |
| A/H3(L)/HA₀-25 | QS21 | 3 ug | 7/10 | 1/10 | PR8 (H1) |

^{a}Amount of peptide in each formulation of peptide-OMPC conjugate

Serum samples were collected and assayed in standard ELISA format as described above.

Elisa Titers

| | | ELISA | SEQ ID |
|---|---|---|---|
| Vaccine | Sequence | titer | NO: |
| A/H1/HA₀-21 | BrAc-GPSIQSRGLFGAIAGFIEE-OH | 9 × 10⁵ | 63 |
| A/H3/HA₀-22 | BrAc-EGPEKQTRGIFGAIAGFIEE-OH | 2 × 10⁷ | 64 |
| A/H1/HA₀-25 | Ac-CEGLRNIPSIQSRGLFGAIAGFIEGGE-OH | 4 × 10⁵ | 61 |
| A/H3(L)/HA₀-25 | Ac-CEGMRNVPEKQTRGLFGAIAGFIENGE-OH | 3 × 10⁷ | 62 |

### EXAMPLE 18

Mouse Challenge Experiment with Influenza Type B Virus in Mice Vaccinated with HA0 Peptides from a Type B Influenza Virus Vonjugated to OMPC.

The influenza B HA₀ conjugate was prepared as described above (see examples above). The conjugation used for the Type B/HA₀-22 EGPAKLLKERGFFGAIAGFLEE (SEQ ID NO:60) peptide-OMPC conjugate was bromoacetyl peptide conjugated to thiolated OMPC.

Female Balb/c mice were immunized intramuscularly with 1, 10, 100 or 1000 ng of B/HA₀-22: (ng based on the peptide content of the conjugate in the formulations) formulated in Merck Alum at weeks 0 and 28. Sera serum samples were collected at weeks 2 and 4 and determined for the HA₀-specific antibody titers by ELISA.

Three weeks after the second immunization, mice were challenged intranasally with LD90 (90% mouse lethal dose) of a mouse adapted influenza B virus, B/Ann Arbor/54. Mice were monitored for survival and weight change thereafter for 20 days.

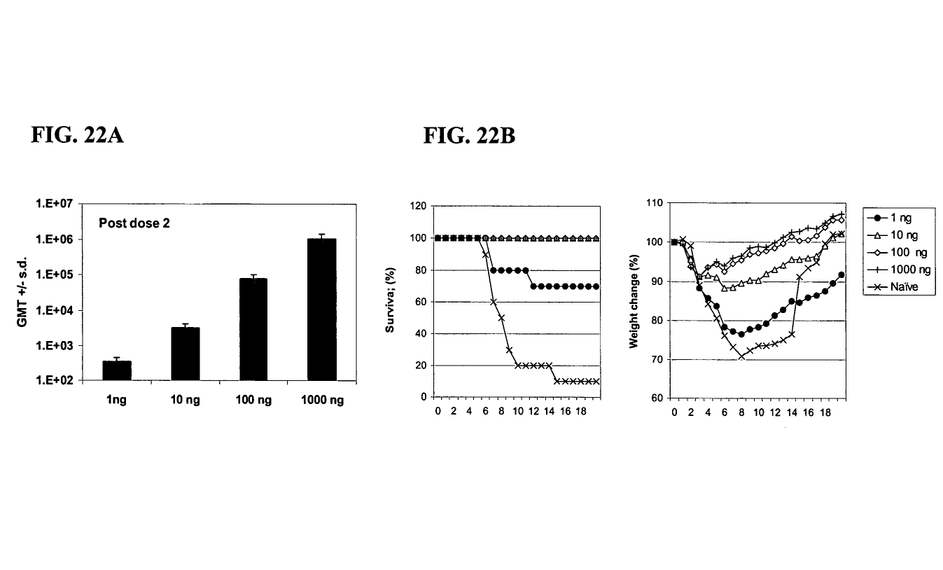

The B/HA₀-OMPC conjugate vaccine elicited potent HA₀-specfic antibody responses (FIG. 22A). The antibody responses were dose-dependent. One ng of the vaccine was able to elicit appreciable HA₀-specific antibody titers, and 1000 ng of the vaccine elicited the titers of approximately 1 million.

The B/HA₀-OMPC conjugate vaccine was also highly effective against lethal virus challenge. As shown in the survival curves (FIG. 22B), mice receiving 10 ng, 100 ng or 1000 ng of the B/HA₀-OMPC vaccine showed 100% survival rate, and mice receiving 1 ng of vaccine had 70% survival rate. The native controls, as expected, showed 90% mortality. The B/HA₀-OMPC vaccine also showed significant protection against weight loss. For example, mice receiving 100 ng or 1000 ng of the vaccine had only 10% maximum weigh loss as compared to the 30% weight loss in control mice.

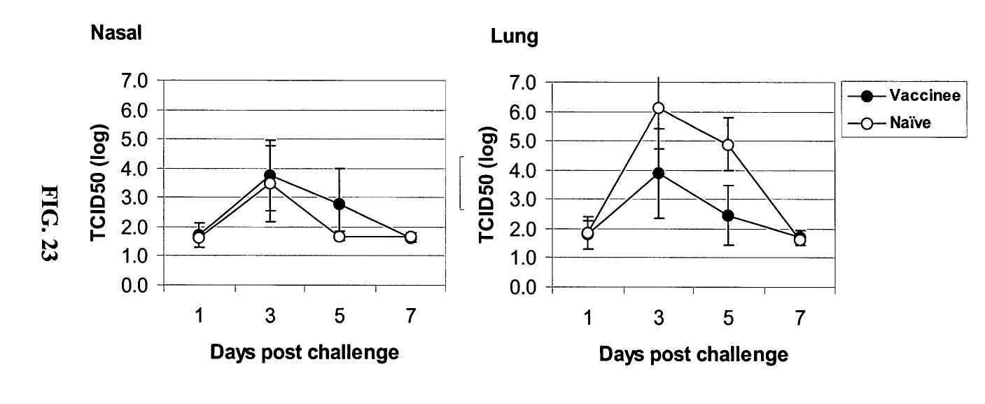

The effects of the influenza B vaccine on in vivo viral replication was tested in a sublethal challenge model. Mice were immunized twice in a four week interval and challenged with sublethal dose of B/Ann Arhor/54. The nasal and lung washes were collected on days 1, 3, 5 and 7. Vaccinees and the controls showed no apparent difference in terms of nasal viral shedding. However, there was significant reduction of lung viral shedding in the immunized mice; comparing with the controls. (FIG. 23.)

### EXAMPLE 19

Mouse Challenge Experiment with Influenza Type A Virus in Mice Vaccinated with A/H3/HA₂ Peptide-KLH Conjugates

The A/H3/HA₂-6-KLH conjugate (KIDLWSYNAELLVALENQHT (SEQ ID NO. 59)) was made by addition of a cysteine residue to the N-terminus of the peptide to provide a thiol group for reaction with maleimide-activated KLH.

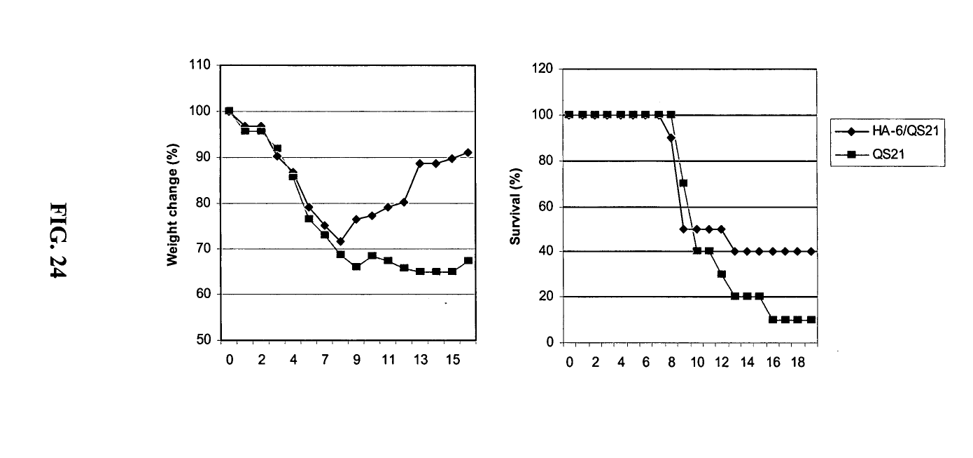

Balb/c mice of 10 per group were immunized with 20 ug of A/H3/HA2-6-KLH conjugate in 20 QS21 subcutaneously at week 0, 3 and 5. Two weeks after the final immunization, mice were challenged intranasally with LD90 of Influenza HK reassortant. HA6-KLH showed partial protection against the lethal challenge. For example, following the challenge, the control group showed 90% mortality whereas the vaccine group showed 60% mortality. In addition, the mice receiving the vaccine showed overall less severe weight loss than did the controls. (FIG. 24)

### EXAMPLE 20

Conjugation of M2 peptide to HPV VLPs

HPV type 16 VLPs were expressed and purified from Saccharomyces cerevisiae as described in (Tobery et al., 2003). The antigen used in this study is a synthetic 25-residue M2-peptide prepared by standard t-Boc solid phase synthesis. The sequence of the peptide is similar to the extra-cellular segment of the M2 protein in Influenza virus strain A/Aichi/470/68 (H₃N₁), Ac-SLLTEVETPIRNEWGSRSNDSSD-Aha-C-NH₂ (SEQ ID NO: 2, and comprises an unnatural amino acid, 6-aminohexanoic acid (Aha).

Antigen-Carrier Conjugation

HPV VLPs in 50 mM NaHCO₃ pH 8.4 at 14 μM in L1 protein concentration were mixed with a commercial heterobifunctional cross-linker 4-(N-maleimidomethyl)-cyclohexane-1-carboxylate (sSMCC) (PIERCE ENDOGEN, ROCKFORT, Ill.) to a final sSMCC/L1 protein (mol/mol) ratio of ˜100. The reaction proceeded for 1 hour at 2-8° C. and was then desalted by dialysis against a pH 6.2 buffer containing 10 mM Histidine, 0.5 M NaCl, 0.015% polysorbate 80 to generate sSMCC-activated HPV VLPs. The maleimide equivalents were determined by the DTNB assay as described in Example 1. The M2-peptide dissolved in N₂-sparged buffer was mixed with sSMCC-activated HPV VLPs to a thiol/maleimide (mol/mol) ratio of ˜3. Alternatively, activated/quenched HPV VLP (A/Q HPV VLP) was prepared by mixing sSMCC-activated HPV VLPs with N-acetylcysteine at a thiol/maleimide (mol/mol) ratio ˜10. The reactions proceeded for ˜15 hours at 2-8° C. Both samples were then treated with β-mercaptoethanol to quench any excess maleimide. Finally, the samples were dialyzed (DISPODIALYSER MWCO 300,000 SPECTRUM INDUSTRIES INC., RANCHO DOMINGUEZ, Calif.) against 0.5 M NaCl and 0.015% polysorbate80. Similar results were obtained when the free thiols in HPV VLPs were quenched with iodoacetamide prior to conjugation.

Determination of Protein Concentration and Peptide Load per VLP

The concentration of protein in solution was determined by a colorimetric bicinchoninic acid (BCA) assay. The peptide load per VLP was determined by amino acid analysis. Samples were hydrolyzed for 70 hours in 6 N HCl at 110° C. and then quantitated after cation-exchange chromatography treatment (AAA SERVICES INC., BORING, OR). The amount of peptide was determined by either referencing to the Aha content or conducting an analysis based on the procedure described by Shuler et al., 1992. Both methods gave similar results.

Antigenic Peptide Loading on the Virus-Like Particle

The peptide load on the HPV VLP was determined using amino acid analysis by either quantitating the unnatural amino acid (Aha, 6-aminohexanoic acid) in the peptide or by multiple regression least-square analysis of data (Shuler et al., “A simplified method for determination of peptide-protein molar ratios using amino acid analysis”, J. Immunol. Meth., Vol. 156 pp. 137-149, 1992). Both methods indicated a peptide loading of about 11 peptides per L1 protein. There are 360 copies of L1 protein in a HPV VLP (a VLP contains 72 L1 protein pentamers or capsomers) thus resulting in a total load of about 4,000 peptide copies per VLP. This number is significantly larger than the previously reported total number of peptides carried on a bovine papillomavirus particle (Chackerian et al., 2001). In the bovine papillomavirus case, an antigenic peptide was fused to streptavidin (SA) and the fusion construct interacted with biotinylated VLPs. The L1 protein of the VLPs was found to accommodate ˜1.5 SA tetramers resulting in a ratio of ˜6 peptides per L1 monomer. This load is about half of that found with our conjugation of M2 peptide to HPV VLP. It is possible that the bulkiness of the SA tetramer precludes a higher antigen loading in the reported case.

The conjugation efficiency can be monitored by determining how many of the initial sites activated by sSMCC resulted in a peptide coupling. Amino acid analysis can provide a quantitative estimation of TXA (tranexamic acid) which is the product of sSMCC cross-linker in the hydrolysis process. The measured average amount of TXA indicated ˜19 activated sites per L1 protein, suggesting that only 58% (or 11/19) of the activated sites were involved in peptide coupling. It is possible that some of the activated sites may interact with proximal side chains of Cys, Lys or His, resulting in cross-linking of the protein. We observed that both M2-HPV VLP and activated/quenched (A/Q) HPV VLPs could not penetrate a 10% SDS-Bis-Tris gel under reducing conditions even with 10-min exposure to denaturing solution treatment at 70° C. Non-activated HPV VLPs present protein bands of the expected mobility after treatment under the same conditions prior to loading to the gel. Therefore it appears that significant intra-VLP cross-linking occurs after maleimide activation. As it will be shown below, VLP size measurements indicate that the impact of inter-VLP cross-linking on the particle size distribution of VLPs is negligible.

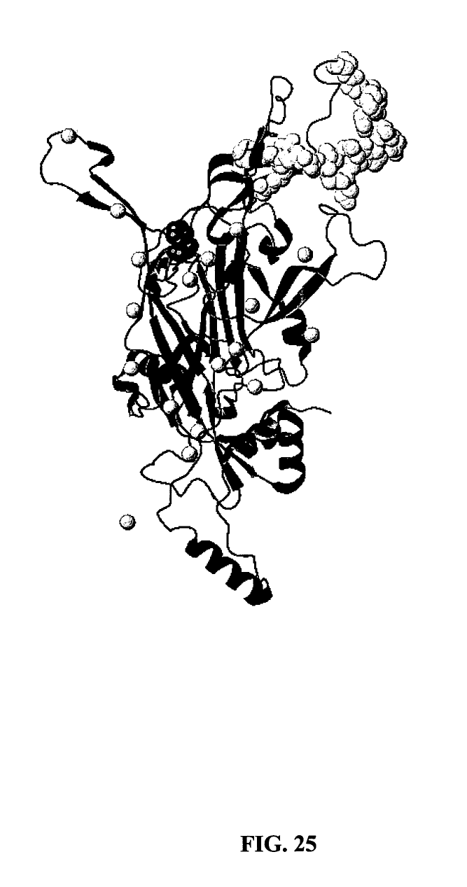

When considering the spatial distribution of the antigenic peptide on the surface of HPV VLPs, the primary amine of the Lys side chain is the most likely site of sSMCC activation. There are 34 Lys in the L1 protein of HPV type 16 and nine of these lysines are located in the C-terminus. The molecular picture shown in FIG. 25 reveals that the putative activation sites on HPV type 16 VLPs are evenly spread on the VLP surface. The NZ atoms of Lys residues presented in FIG. 25 are oriented towards the exterior of the VLP. Except for Lys 230, all Lys residues have more than 25% of the surface exposed to the solvent. The C-terminus region is very flexible and accessible to proteases, so it is very probable that the side chains of Lys situated in this region are available for activation. Unfortunately, the C-terminus region was not resolved in the X-ray structure (Chen et al., 2000).

### EXAMPLE 21

Pharmaceutical Characterization of M2-HPV VLP Conjugates

Electron microscopy measurements were performed by ELECTRON MICROSCOPY BIOSERVICES (MONROVIA, Md.) using a JEOL 1200 EX Transmission Electron Microscope at high magnification. Air-dried samples were stained with 2% phosphotungstic acid. Dynamic light scattering measurements were performed on a Malvern 4700 instrument with detection at 90° and room temperature. The output power was at 0.25 W, aperture of 100 and total protein concentration of 0.1 mg/mL. The size reported represents the Z-average hydrodynamic diameter as resulted from monomodal analysis of data obtained in five consecutive measurements on the same sample. The heat-induced increase in the turbidity of HPV VLP or M2-HPV VLP conjugate solutions was monitored on a spectrophotometer HP 8453 equipped with a thermal controller type 89090A. The variation in optical density at 350 nm was recorded as the temperature increased from 24° C. to 74° C. at rate of ˜1.5° C./min. Sedimentation velocity experiments were performed on an analytical ultracentrifuge Beckman XL-I using a rotor An6Ti and a double-sector cell. The rotor speed was 10,000 rpm and the boundary movement was observed by absorption at 280 nm. Data was analyzed using the program DCDT+(http://www.jphilo.mailway.com). SEC-HPLC was performed on a HP 1100 System equipped with a Shodex OHpak SB-805 column and an elution buffer containing 25 mM phosphate, 0.75 M NaCl pH 7.0.

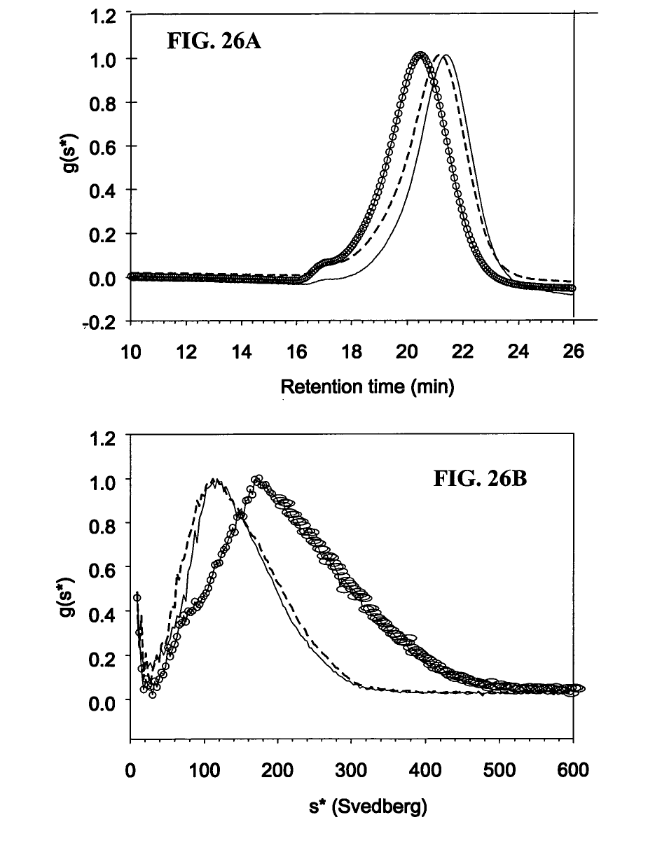

Dynamic light scattering (DLS) measurements indicate a slight increase in the average particle size of the M2-HPV VLP conjugate, from ˜60 nm for the untreated HPV VLP carrier to ˜80 nm for the conjugate (M2-HPV VLP). The A/Q HPV VLPs reveal an average hydrodynamic size of ˜65 nm, a value that is very close to the size of the untreated carrier. SEC-HPLC results (FIG. 26A) present the main peak of M2-HPV VLP conjugate eluting at shorter retention time compared to A/Q or untreated HPV VLPs; that corresponds to a particle size of the conjugate larger than that of A/Q or untreated HPV VLPs. The small shoulders in the chromatograms reveal the presence of a small fraction of aggregated material before and after the conjugation. Finally, sedimentation velocity data (FIG. 26B) presents a distribution of sedimentation coefficients for the M2-HPV VLP centered at s* values larger than that of the untreated or A/Q HPV VLPs. The slight increase of the sedimentation coefficient of conjugate compared to carrier alone is consistent with a small size increase upon conjugation as revealed by DLS and chromatographic measurements. The overall results also suggest that no significant inter-VLP cross-linking (and, implicit, aggregation) occurs during the conjugation process.

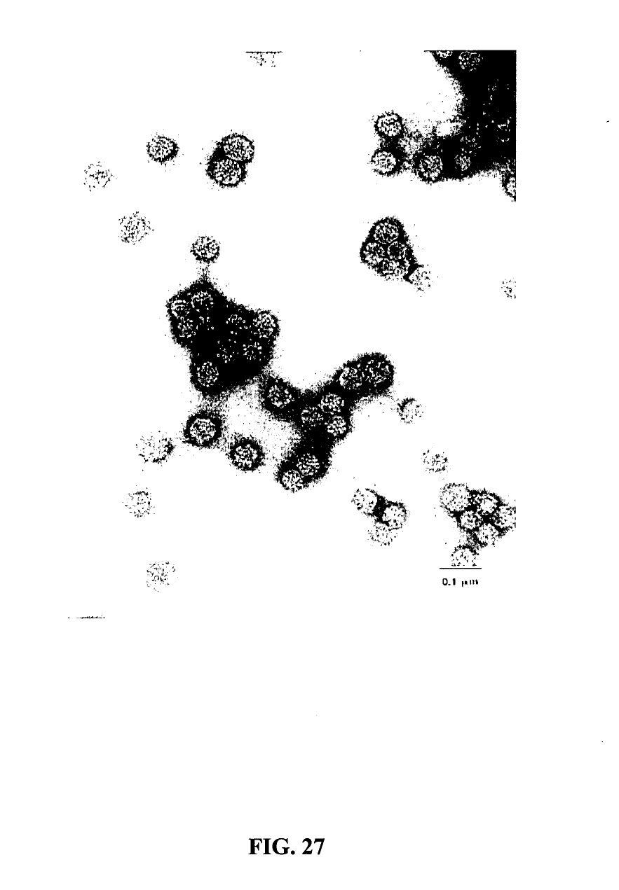

The M2-HPV VLP conjugates observed by EM (FIG. 27) present a size distribution between 40 to 95 nm, with a mean at approximately 65 nm. This value is very close to that of the untreated HPV VLPs. However, in contrast with the unconjugated carrier, the conjugates were found to have a “fuzzy appearance” in M2-HPV VLP, which may be due to the presence of conjugated peptide. The multi-VLP aggregates shown in EM images are observed for HPV VLP as well; therefore they may be the result of sample manipulation for EM measurement and are not representative for the sample in solution. In conclusion, EM results support that the morphology of HPV VLPs was preserved and that no major disruption of HPV VLP scaffold occurred during the chemical conjugation process.

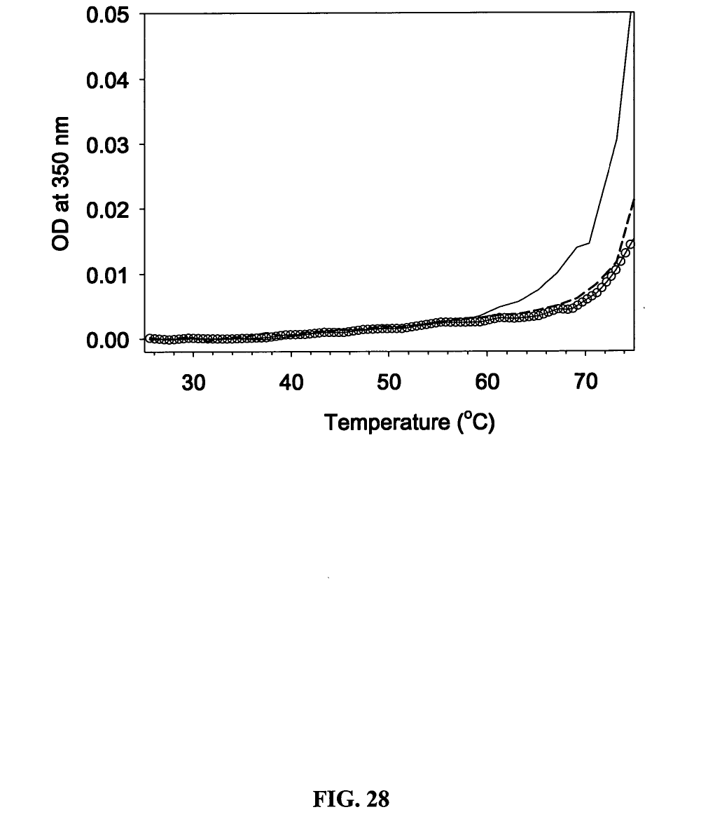

The profiles of heat-induced aggregation determined by a solution turbidity assay for treated and untreated HPV VLPs or the conjugates are shown in FIG. 28. For untreated HPV VLPs, the heat-induced aggregation (as revealed by the increase in optical density due to light scattering) becomes detectable at 60° C. and increases in an abrupt manner if the temperature is further increased. For the A/Q VLPs or M2-HPV VLP conjugates the turbidity of solution does not present detectable aggregation below 70° C. It is very likely that the enhanced stability against heat-induced aggregation is due to the intra-VLP cross-linking induced by sSMCC treatment. The additional intra-VLP bonds formed via sSMCC may prevent L1 protein from partial unfolding and subsequent exposure of hydrophobic surfaces. It is worth noting that the conjugation or sSMCC treatment resulted in the change of the surface properties of the HPV VLPs, which may in part contribute to the stability enhancement of the carrier.

### EXAMPLE 22

In Vitro Antigenicity Analysis of M2-HPV Conjugates

Detection of Conjugate Interactions with Anti-HPV and Anti-M2 Antibodies

The binding of HPV type 16 VLPs and M2-HPV VLP conjugates to antibodies specific to M2 or HPV type 16 was evaluated using the surface plasmon resonance technique on a Biacore 2000 instrument. The anti-HPV antibodies (conformational antibodies H16.V5, H16.E70 and linear epitope binding antibody H16.J4) and anti-M2 antibodies were bound to rat anti-mouse Fcγ antibody chemically immobilized on the surface of a sensor chip type CM5.

The spatial distribution of antigen was further investigated by determining the binding of M2-HPV VLP conjugate and A/Q HPV VLP to linear and conformational anti-HPV mouse antibody (mAB). The binding affinity for the conformational or neutralization antibodies H16.V5 and H16.E70 was found to be dramatically decreased, while the binding to linear antibody H16.J4 was only slightly affected upon conjugation. The epitopes involved in the binding of the conformational antibodies H16.V5 and H16.E70 comprise Phe 50 (White et. al., “Characterization of a Major Neutralizing Epitope on Human Papillomavirus Type 16 L1”, J. Virol., Vol. 73 (6), pp. 48824889, 1999). As shown in FIG. 25, there are 6 Lys residues, which flank Phe 50. It is likely that conjugation of a peptide to any of the Lys residues around Phe 50 will perturb the antibody binding. H16.J4 binds to a loop on the top of L1 protein in VLP. There is only one Lys along this loop, which may not become conjugated with peptide because the binding to H16.J4 is not altered in M2-HPV VLP.

One concern is whether the peptide is presented in the correct 3-D configuration on the surface of the carrier. The M2 protein is an integral membrane protein of the Influenza A virus and the antigenic sequence selected represents the extracellular part of M2. The M2 protein is a homotetramer formed by two disulfide-linked dimers (Tian et al., “Initial structural and dynamic characterization of the M2 protein transmembrane and amphipathic helices in lipid bilayers”, Prot. Sci., Vol. 12, pp. 2597-2605, 2003) and, to our knowledge, no detailed 3D-structure was reported in the literature about the extracellular segment of M2. CD and fluorescence measurements suggest that the unconjugated peptide in solution is predominantly in random structural configuration. Although these findings disfavor presentation of the peptide in a defined structural configuration on the surface of VLP, preliminary results obtained by surface plasmon resonance indicate that the M2-HPV VLP conjugate binds to anti-M2 antibodies L18.H12 and P6.C8. No binding to anti-M2 antibodies was detected under similar conditions with HPV VLPs or (A/Q) HPV VLP.

### EXAMPLE 23

In Vivo Immunological Evaluation

Four to ten week female Balb/c mice were obtained from CHARLES RIVER LABORATORIES (Wilmington, Mass.). M2-HPV VLP adsorbed on Merck Aluminum Adjuvant (MAA) at different peptide doses was delivered by 0.1 mL I.M. in two injections four weeks apart. The mice were challenged 3 weeks after the second injection. The peptide doses of 3, 30 and 300 ng correspond to about 5, 50 and 500 ng of HPV VLP. The dose of MAA delivered at each injection was 45 mcg. Anti-M2 geometric mean titers were determined at 2 weeks after each injection. For M2 antibody ELISA, 96-well plates were coated with 50 μl per well of M2 peptide at a concentration of 4 μg per ml in 50 mM bicarbonate buffer, pH 9.6, at 4° C. over night. Plates were washed with phosphate buffered saline (PBS) and blocked with 3% skim milk in PBS containing 0.05% Tween-20 (milk-PBST). Testing samples were diluted in a 4-fold series in PBST. One hundred μl of a diluted sample was added to each well, and the plates were incubated at 24° C. for 2 hour and then washed with PBST. Fifty μl of predetermined dilutions of HRP-conjugated secondary antibodies in milk-PBST was added per well and the plates were incubated at 24° C. for 1 hr. Plates were washed and 100 μl of 1 mg/ml o-phenylenediamine dihydrochloride in 100 mM sodium citrate, pH 4.5 was added per well. After 30 min incubation at 24° C., the reaction was stopped by adding 100 μl of 1N H₂SO₄ per well, and the plates were read at 490 nm using an ELISA plate reader. The antibody titer was defined as the reciprocal of the highest dilution that gave an OD490 nm value above the mean plus two standard deviations of the conjugate control wells. For viral challenge, mouse adapted viruses A/Puerto Rico/8/34 (PR8; H1N1) and X-31(H₃N₂), a reassortant between PR8 and A/Aichi/68 (H₃N₂), were propagated in allantoic fluid of 10 day-old embryonated eggs. The mice were anesthetized with ketamine/xylazine. Twenty microliter of virus with 1 LD90 was instilled into nostrils. After challenge, the mouse survival rate were recorded daily. The mortality rate was calculated as: (number of mice at the day specified/number of mice at day 0)×100%.

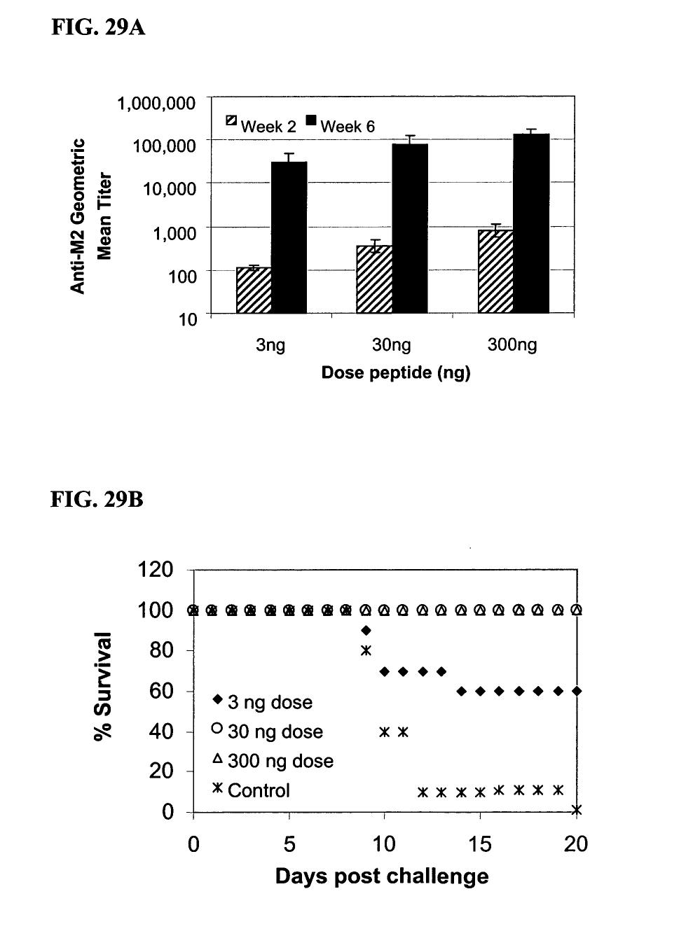

Results of ELISA measurements on blood samples taken two weeks after each immunization indicate that the conjugate elicited high anti-M2 antibody response (FIG. 29A). Although the titers increase in a systematic manner as the M2 peptide dose is increased from 3 to 300 ng, the difference in titers between the lowest and highest dose is within one log unit. These results indicate that the antigenic peptide at nanogram doses can induce a significant immune response when presented on a suitable carrier. It is worth noting that similar titers are observed in mice when the M2 peptide is conjugated on a larger-size carrier, the Neisseria meningitidis outer-membrane protein complex (OMPC) as described above.

The survival rates of mice against lethal challenge are shown in FIG. 29B. The group receiving the lowest dose of peptide (3 ng) shows only 60% survival, whereas the protection in groups with higher doses of 30 or 300 ng peptide is 100%. No survival after challenge was observed for the control group, confirming that the virus challenge and the vaccine protection were both effective. As seen above for the M2-OMPC conjugate vaccines, some weight loss was observed after challenge even in the groups with 100% survival. In conclusion, the vaccination of Balb/c mice with M2-HPV VLP conjugate vaccine efficiently protects the animals against live virus challenge.

The carrier-induced epitope-specific suppression has been described in literature (Rauly et al., 1999). Therefore, future experiments should determine how the immunogenicity of the conjugate is affected by the presence of anti-carrier antibodies in vivo. The experiment presented in Example 26 with M2-OMPC conjugate vaccines indicates that pre-exposure to carrier did not abolish, but only slightly diminished the response to the influenza peptide conjugate vaccine. However, it was suggested that subsequent boosts could overcome any detrimental effect of pre-existing antibodies against the carrier.

Despite the overwhelming number of cases in which preimmunization with a carrier was shown to impair the antibody response, one cannot simply propose a priori that the presence of anti-carrier antibodies has an adverse effect on the immunogenicity of a conjugate vaccine. It was reported that prior immunity to carrier (tetanus toxoid) was beneficial either to anti-hCG (human chorionic gonadotropin, (Shah et al., “Prior immunity to a carrier enhances antibody responses to hCG in recipients of an hCG-carrier conjugate vaccine”, Vaccine, Vol. 17, pp. 3116-3123, 1999) or to malarial peptide (Lise et al., “Enhanced epitopic response to a synthetic human malarial peptide by preimmunization with tetanus toxid carrier”, Infect. Immun., Vol. 55, pp. 2658-2661, 1987) response. In a different case describing recombinant flagella as a carrier of influenza peptide epitopes it was found that there was no effect of preexposure to carrier (Ben-Yedidia and Arnon, “Effect of pre-existing immunity on the efficacy of synthetic influenza vaccine’, Immunol. Lett., Vol. 64, pp. 9-15, 1998). It has not yet been determined, in the case of HPV VLPs, whether there is any difference in animal models pre-exposed to the carrier in the untreated form (as an anti-HPV vaccine) or the treated form (as a carrier presenting a different antigen). It was found that more than 75% of reactive human sera were completely blocked by H16.V5 antibody (Wang et al., “A monoclonal antibody against intact human papillomavirus type 16 capsids blocks the serological reactivity of most human sera”, J. Gen. Virol., Vol. 78, pp. 2209-2215, 1997). The fact that conjugated M2-HPV VLP does exhibit the conformational epitope bound by the H16.V5 antibody suggests that carrier suppression to vaccines prepared through chemical conjugation between antigen and HPV VLPs as carrier would not be a major concern for those who were pre-exposed to HPV.

Experiments with M2-OMPC shown herein have demonstrated that the protection against influenza virus lethal challenge can be passively transferred by the administration of immunized animal sera, indicating that neutralizing antibodies were sufficient to confer protection. Because the same antigen was conjugated to the HPV carrier, it is expected that a similar humoral response was triggered by the immunization with M2-HPV VLP conjugate. In regard to the cellular response, previous experiments showed that HPV type 16 VLPs induced a strong Th2 response as measured by CD4+ T cells production of IL4 (Tobery et al., “Effect of vaccine delivery system on the induction of HPV16 L1-specific humoral and cell-mediated immune responses in immunized rhesus macaques”, Vaccine, Vol. 21, pp. 1539-1547, 2003). It was also proposed that non-conformational antigenic sequences presented by HPV VLPs might enhance the cell-mediated immune response (Greenstone et al., 1998).

### EXAMPLE 24

Conjugation of a Hemagglutinin-Derived Peptide to VLP

Peptide Cys-A/H3/HA0-22 was conjugated to an HPV VLP.

| SEQ ID | | | |
|---|---|---|---|
| NO: | Name | Peptide Sequence | MW |
| 113 | Cys-A/H3/HA0-22 | Ac-CEGPEKQTRGIFGAIAGFI | 2293 |
| | | EE-OH | |

The peptide sequence of Cys-A/H3/HA0-22 corresponds to the region spanning the cleavage site of the Hemagglutinin protein precursor HA₀ of Influenza A consensus sequence, H3 subtype. Indicated in bold are residues required to accomplish different functions, respectively at the N-terminus: a Glycine as a spacer, a Glutamic acid as a pI-modifying group (as described herein), and a Cysteine as a ligand to react with a maleimide activated HPV VLP carrier to generate the peptide-VLP conjugate via a thioether linkage; at the C-terminus: a glutamate as a pI-modifying group.

Peptide Synthesis of Cys-A/H3/HA₀-22

The peptide was synthesized by solid phase using Fmoc/t-Bu chemistry on a PIONEER Peptide Synthesizer (APPLIED BIOSYSTEMS, FOSTER CITY, Calif.). To produce the peptide C-terminal acid, the peptides were synthesized on a CHAMPION PEG-PS resin (BIOSEARCH TECHNOLOGIES, INC, NOVATO, Calif.) that had been previously derivatized with the 4-hydroxymethylphenoxyacetic acid linker using DIPCDI/HOBt as activators. The first amino acid, Glutamate, was activated as symmetrical anhydride with DIPC (diisopropylcarbodiimide) and esterified to the resin in the presence of a catalytic amount DMAP (dimethylaminopirydine). The acetylation reaction was performed at the end of the peptide assembly by reaction with a 10-fold excess of acetic anhydride in DMF.

All the acylation reactions were performed for 60 min with 4-fold excess of activated amino acid over the resin free amino groups. Amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropylethylamine). The general side chain protecting group scheme was: tert-butyl for Asp, Glu, Ser, Thr and Tyr; trityl for Cys, Asn, His and Gln; 2,2,4,6,7-pentamethyldihydrobenzofuran-5-sulfonyl for Arg; tert-butoxy-carbonyl for Lys, Trp. At the end of the assembly, the dry peptide-resin was treated with 88% TFA, 5% phenol, 2% triisopropylsilane and 5% water (Sole, N. A., and Barany, G. (1992) J. Org. Chem., 57, 5399-5403) for 1.5 h at room temperature.

The resin was filtered and the solution was added to cold methyl-t-butyl ether in order to precipitate the peptide. After centrifugation the peptide pellets were washed with fresh cold methyl-t-butyl ether to remove the organic scavengers. The process was repeated twice. The final pellets were dried, resuspended in H₂O, 20% acetonitrile and lyophilized.

The crude peptide was purified by reverse-phase HPLC using a semi-preparative RCM DELTA-PAK™ (WATERS, MILFORD, Mass.) C₋₁₈ cartridges (40×200 mm, 15 μm) using as eluents (A) 0.1% trifluoroacetic acid in water and (B) 0.1% trifluoroacetic acid in acetonitrile. We used the following gradient of B: 30%-45% over 20 min, flow rate 80 ml/min. Analytical HPLC was performed on a ULTRASPHERE (BECKMAN, FULLERTON, Calif.), C₁₈ column, 25×4.6 mm, 5 μm with the following gradient of B: 30%-45% B in 20 minutes, flow 1 ml/min. The purified peptide was characterized by electrospray mass spectrometry on a PERKIN-ELMER (WELLESLEY, Mass.) API-100: theoretical average mw is 2293.4 Da, measured was 2293.8 Da. Conjugation of peptide Cys-A/H3/HA₀-22 to HPV VLP

HPV VLP 16 sterile stock solution was produced at a concentration of 0.869 mg/ml in 0.5M NaCl, 20 mM His buffer, 0.026% PS80 at pH 6.2. An aliquot of HPV VLP stock solution, 2.5 mL, was dialyzed at 4° C. using 300K MWCO DISPODIALYZER (SPECTRUM LABORATORIES, INC., RANCHO DOMINGUEZ, Calif.) with 6-buffer changes (every 2 h) of 2 L, of 0.5 M NaCl, 0.026 PS80, in order to remove the His buffer which might interfere with the activation reaction. To the HPV VLP solution (0.474 mg/mL, 4.58 mL) was added was added 0.5 M NaHCO₃ (0.506 mL) to a final concentration of 50 mM, pH 8.2. To this was added drop-wise 0.156 mL of a 20 μM solution of the heterobifunctional crosslinker sulfosuccinimidyl 4-(N-maleimidomethyl)cyclohexane-1-carboxylate (sSMCC, PIERCE CHEMICAL CO, ROCKFORD, Ill.), which corresponds to a 4-fold excess over the available VLP lysine residues. After aging the solution for 2 hour in the dark at 4° C., the activated HPV VLP was dialyzed at 4° C. using 300K MWCO DISPODIALYZER (SPECTRUM LABORATORIES, INC., RANCHO DOMINGUEZ, Calif.) with 6-buffer changes (every 2 h at least) of 2 L, of 10 mM His buffer, 0.5 M NaCl, 0.015% PS80, pH 6.2 to remove excess reagents. A total of 6.1 mL, 0.356 mg/ml of activated HPV VLP (aVLP) was recovered after dialysis.

A 0.5 mg/ml stock solution of the Cys-containing peptide ligand Cys-A/H3/HA0-22, was prepared in degassed solution of 0.1 M His, 0.5 M NaCl, 0.015% PS80 pH 7.2 and 0.2μ filtered. The thiol content of the peptide solution was determined by the Ellman assay (Ellman, G. L. (1959), Arch. Biochem. Biophys., 82, 70) and showed a —SH titre of 218 μM.

To define the maximum amount of peptide ligand that could be safely incorporated on aVLP without causing precipitation, the conjugation reaction was first followed in small-scale trials where the aVLP was incubated with increasing amounts of peptide ligand. The maximum number of maleimide groups that can be incorporated on a VLP is limited by the number of lysine residues displayed on its exterior surface which are therefore available for chemical modification. Based on the X-ray structure of L1 protein there are 0.36 μmoles lysine/mg VLP available for conjugation. If one considers an average MW of 20×10⁶ Da for VLP, this corresponds to 7,200 lysine moles/VLP mol. Therefore aVLP was incubated with the following molar excesses of peptide ligand per VLP mol: 1000, 2000, 4000, 6000. After one hour, the samples were compared with an aVLP sample to check for the presence of any precipitation or turbidity. The conjugation reaction gave a soluble product only when using a molar excess up to 1000 (of moles Cys-peptide/VLP mol) for the 1 hour incubation reaction. Above that ratio, a complete precipitation of the VLP solution occurred.

On the basis of these observations a large-scale reaction was performed: 3.5 mL (1.25 mg) in 10 mM His, 0.5M NaCl, was added 56 μL of NaOH 0.25 M to raise the pH to 7.2. To this was added 0.28 mL of the peptide stock solution, drop-wise while gently vortexing, which corresponds to 1000 molar excess of peptide moles/VLP mol. A sample of maleimide-activated VLP solution was retained as blank for the determination of the peptide loading of the final conjugate. The conjugation reaction mixture was allowed to age for 17 h at 4° C. in the dark. Any residual maleimide groups on the VLP were then quenched with β-mercaptoethanol to a final concentration of 15 mM (4 μL total volume added) for 1 h at 4° C. in the dark. The solution was dialyzed 4 times, 5 hour/change, with 1 L of 0.5M NaCl, 0.015% PS80 at 4° C. with 300K MWCO DISPODIALYZER (SPECTRUM LABORATORIES, INC., RANCHO DOMINGUEZ, Calif.) to remove unconjugated peptide and β-mercaptoethanol. The concentration was determined by BCA-assay (PIERCE CHEMICAL CO., ROCKFORD, Ill.), revealing 0.131 mg/mL (4.5 mL) for the VLP-A/H3/HA₀-22.

The conjugate and a aOMPC samples were hydrolyzed in evacuated, sealed glass tubes with azeotropic HCl for 70 hours at 110° C. The amino acid composition was determined by amino acid analysis. The conjugation load of peptide to OMPC protein was determined by comparing the conjugate amino acid composition with both that of the VLP carrier and that of peptide ligand and by multiple regression, least squares analysis of the data (Shuler et al., J. Immunol. Meth., 156, (1992) 137-149). For the conjugate between VLP and A/H3/HA₀-22, a molar ratio of 770 was obtained (peptide/VLP mol/mol).

### EXAMPLE 25

Inhibition of Viral Shedding by M2 Conjugate Vaccine

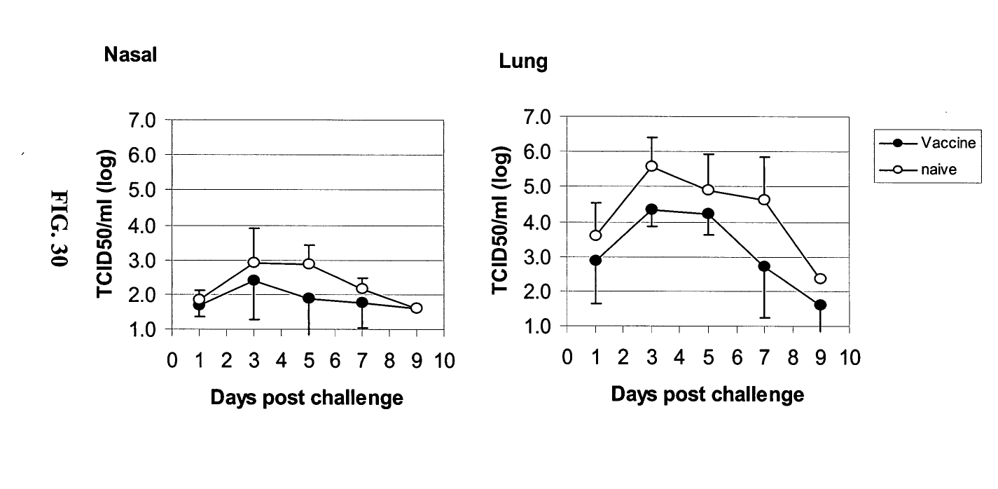

An M2-KLH conjugate vaccine, prepared with M2 peptide SEQ ID NO: 1 as described in Example 5, was evaluated for its effects on viral replication in the mouse respiratory tract (FIG. 30). Balb/c mice per group were immunized intramuscularly with 20 μg of conjugate vaccine M2-KLH plus 20 μg of QS21 (M2-KLH/QS21) or 20 μg QS21 only (QS21) on days 0, 14 and 28. Three weeks after the third immunization, mice were challenged intranasally with 75 TCID50 of A/HK/68 reassortant. Following the challenge, eight mice from each group were sacrificed at day 1, 3, 5, 7 or 9, to collect nasal and lung washes. The viral titers at the respective time points were determined. Immunized mice had overall lower viral titers in both nasal and lung samples than the control mice. The reduction of viral shedding was more pronounced in the lungs. The difference in viral shedding in the lung between control and the vaccinees was statistically significant (p<0.05).

### EXAMPLE 26

Immunogenicity of M2 Conjugate Vaccine in Rhesus Monkeys

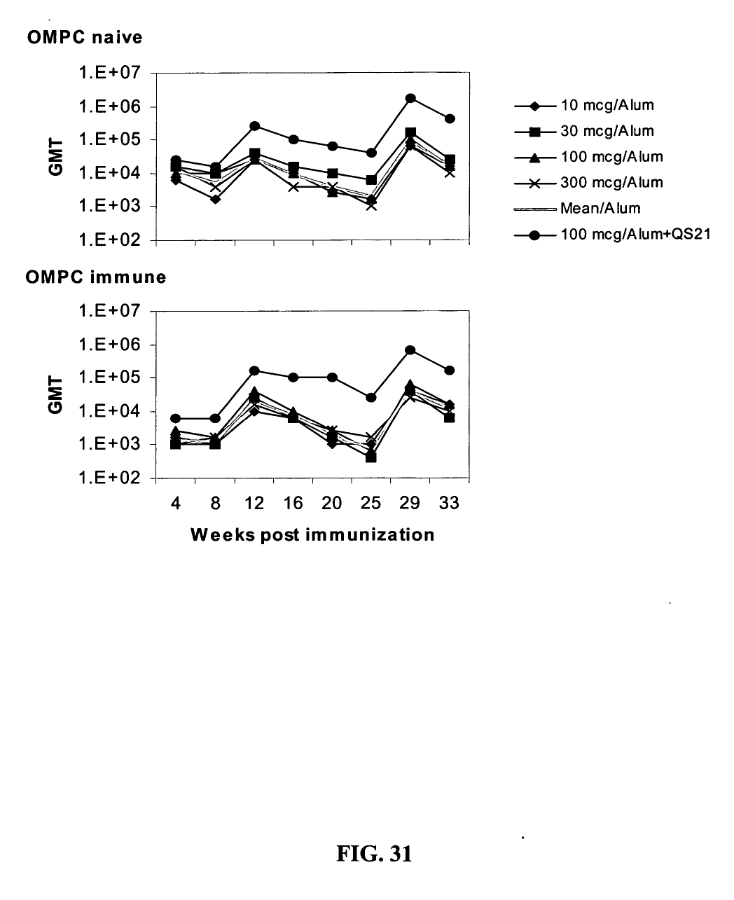

An M2-OMPC conjugate made with M2 peptide SEQ ID NO: 2, prepared as in Example 5, was tested in both naïve and OMPC-immune rhesus monkeys (FIG. 31). OMPC has been used as the carrier for several bacterial polysaccharide conjugate vaccines, including a licensed Haemophilus Influenza vaccine (PEDVAXHIB, MERCK & CO., INC., WEST POINT, Pa.). Therefore, this experiment tested whether pre-existing immunity to OMPC would overtly affect the flu vaccine potency.

Thirty monkeys were divided into two groups of fifteen monkeys each. One group was pre-immunized with two human doses of PEDVAXHIB in order to induce an anti-OMPC antibody response. The monkeys that had received the PEDVAXHB immunization developed OMPC GMTs of 14,703 six weeks prior to M2-OMPC immunization.

The OMPC-immunized monkeys and the naive monkeys were then each divided into five groups of three monkeys each, and immunized intramuscularly with 10 μg, 30 μg, 100 μg and 300 μg of the M2-OMPC conjugate vaccine (dose based on total conjugate protein) formulated in Alum, or 100 μg of the vaccine formulated in Alum plus QS21. The immunizations were performed using a 0-, 8- and 25-week schedule. Blood samples were collected at four to five week intervals for thirty-three weeks.

The M2-OMPC vaccine elicited significant M2-specific titers after a single immunization. These responses were further boosted after a second and third immunization. In both the OMPC-immunized and the OMPC-naive monkeys there was no apparent dose effect, with the lowest dose, 10 μg, eliciting M2-specific titers comparable to those elicited by the highest dose, 300 μg. The vaccine formulated in Alum plus QS21 showed 5 to 10-fold higher antibody titers than the same dose of the conjugate formulated in Alum alone. In addition, antibody titers in monkeys that received the vaccine in Alum plus QS21 appeared to have a slower decline rate than that observed in the monkeys that received vaccine in Alum alone.

When comparing OMPC-immunized and OMPC-naïve monkeys, the former showed approximately 10-fold lower titers than did the naïve monkeys after the first injection. This indicated that the pre-existing antibody to the carrier does have a negative effect on the immunogenicity of the M2-OMPC conjugate vaccine. However, the detrimental effect of preexisting immunity to the carrier was overcome by subsequent boosts. After the second and the third immunization the groups in the two arms of the study reached comparable anti-M2 titers. The results therefore show that the M2-OMPC vaccine is immunogenic in nonhuman primates, either with or without pre-existing antibodies to the carrier. In a separate monkey study, we also tested a regimen involving co-administration of PEDVAXHIB and M2-OMPC conjugate vaccine, and found no negative effect on the overall antibody responses to the M2 peptide. Therefore, this vaccine can be used in the populations with prior exposure to other OMPC-based conjugate vaccines.

What is claimed:

1. An M2 peptide-protein conjugate comprising a plurality of peptides having an amino acid sequence derived from the extracellular domain of the M2 protein of Influenza virus type A, said plurality of peptides being covalently linked to the surface of a carrier protein and each said linkage being between one terminus of a peptide and a reactive site at the surface of said protein, wherein the carrier protein is selected from the group consisting of the outer membrane protein complex of Neiserria meningitidis, tetanus toxoid, Hepatitis B Surface Antigen, keyhole limpet hemocyanin, a Rotavirus capsid protein, and the L1 protein of a bovine or human Papilloma Virus VLP, or a pharmaceutically acceptable salt thereof.

2. The conjugate of claim 1 wherein the amino acid sequence of the peptides is selected from the group consisting of SEQ ID NOs: 1, 2, 10 and 39.

3. The conjugate of claim 2 wherein said peptide has the sequence of SEQ ID NO: 39

4. The conjugate of claim 1 wherein said carrier protein is the outer membrane protein complex of Neiserria meningitidis.

5. The conjugate of claim 4 wherein said peptide has the amino acid sequence of SEQ ID NO: 39 and said immunogenic protein is the outer membrane protein complex of Neiserria meningitidis.

6. The conjugate of claim 1 wherein the peptide is covalently linked to the protein via a thioether linker.

7. A vaccine for the prevention or amelioration of infection of a mammal by influenza virus type A comprising at least one peptide-protein conjugate of claim 1, an adjuvant and a physiologically acceptable carrier.

8. The vaccine of claim 7 wherein the adjuvant comprises an aluminum containing adjuvant.

9. The vaccine of claim 7 wherein the adjuvant comprises aluminum and QS21.

10. The vaccine of claim 7 wherein said peptide-protein conjugate comprises a plurality of peptides having the amino acid sequence of SEQ ID NO: 39 and said protein is the outer membrane protein complex of Neiserria meningitidis.

11. A method of inducing an immune response in a patient comprising the step of inoculating a patient with an effective amount of a conjugate of claim 1.

12. The method of claim 11 wherein the patient is a human.

13. An HA₀ peptide-protein conjugate comprising a plurality of peptides having an amino acid sequence derived from the HA₀ protein of Influenza type A virus, said plurality of peptides being covalently linked to the surface of a carrier protein and each said linkage being between one terminus of a peptide and a reactive site at the surface of said protein, or a pharmaceutically acceptable salt thereof.

14. The conjugate of claim 13 wherein the amino acid sequence of the peptides is selected from the group consisting of SEQ ID NOs: 59, 60, 61, and 62.

15. The conjugate of claim 14 wherein said peptide has the sequence of SEQ ID NO: 62

16. The conjugate of claim 13 wherein said carrier protein is selected from the group consisting of the outer membrane protein complex of Neiserria meningitidis, tetanus toxoid, Hepatitis B Surface Antigen, Hepatitis B Core Antigen, keyhole limpet hemocyanin, a Rotavirus capsid protein, and the L1 protein of a bovine or human Papilloma Virus VLP.

17. The conjugate of claim 16 wherein said peptide has the amino acid sequence of SEQ ID NO: 62 and said immunogenic protein is the outer membrane protein complex of Neiserria meningitidis.

18. The conjugate of claim 13 wherein the peptide is covalently linked to the protein via a thioether linker.

19. A vaccine for the prevention or amelioration of infection of a subject by influenza type A virus comprising at least one peptide-protein conjugate of claim 13, an adjuvant and a physiologically acceptable carrier.

20. The vaccine of claim 19 wherein the adjuvant comprises an aluminum containing adjuvant.

21. The vaccine of claim 19 wherein the adjuvant comprises aluminum and QS21.

22. The vaccine of claim 19 wherein said peptide-protein conjugate comprises a plurality of peptides having the amino acid sequence of SEQ ID NO: 62 and said protein is the outer membrane protein complex of Neiserria meningitidis.

23. A method of inducing an immune response in a patient comprising the step of inoculating a patient with an effective amount of a conjugate of claim 13.

24. The method of claim 23 wherein the patient is a human.

25. An HA₀ peptide-protein conjugate comprising a plurality of peptides having an amino acid sequence derived from the HA₀ protein of Influenza type B virus, said plurality of peptides being covalently linked to the surface of a carrier protein and each said linkage being between one terminus of a peptide and a reactive site at the surface of said protein, or a pharmaceutically acceptable salt thereof.

26. The conjugate of claim 25 wherein the amino acid sequence of the peptides is selected from the group consisting of SEQ ID NOs: 60, 126-168.

27. The conjugate of claim 26 wherein said peptide has the sequence of SEQ ID NO: 60.

28. The conjugate of claim 25 wherein said carrier protein is selected from the group consisting of the outer membrane protein complex of Neiserria meningitidis, tetanus toxoid, Hepatitis B Surface Antigen, Hepatitis B Core Antigen, keyhole limpet hemocyanin, a Rotavirus capsid protein, and the L1 protein of a bovine or human Papilloma Virus VLP.

29. The conjugate of claim 28 wherein said peptide has the amino acid sequence of SEQ ID NO: 60 and said immunogenic protein is the outer membrane protein complex of Neiserria meningitidis.

30. The conjugate of claim 25 wherein the peptide is covalently linked to the protein via a thioether linker.

31. A vaccine for the prevention or amelioration of infection of a subject by influenza type B virus comprising at least one peptide-protein conjugate of claim 25, an adjuvant and a physiologically acceptable carrier.

32. The vaccine of claim 31 wherein the adjuvant comprises an aluminum containing adjuvant.

33. The vaccine of claim 31 wherein the adjuvant comprises aluminum and QS21.

34. The vaccine of claim 31 wherein said peptide-protein conjugate comprises a plurality of peptides having the amino acid sequence of SEQ ID NO: 60 and said protein is the outer membrane protein complex of Neiserria meningitidis.

35. A method of inducing an immune response in a patient comprising the step of inoculating a patient with an effective amount of a conjugate of claim 25.

36. The method of claim 35 wherein the patient is a human.

37. A vaccine for the prevention or amelioration of infection of a patient by influenza virus comprising at least one peptide-protein conjugate of claim 1, at least one peptide-protein conjugate of claim 13, an adjuvant and a physiologically acceptable carrier.

38. A vaccine for the prevention or amelioration of infection of a patient by influenza virus comprising at least one peptide-protein conjugate of claim 1, at least one peptide-protein conjugate of claim 25, an adjuvant and a physiologically acceptable carrier.

39. A vaccine for the prevention or amelioration of infection of a patient by influenza virus comprising at least one peptide-protein conjugate of claim 1, at least one peptide-protein conjugate of claim 13, at least one peptide-protein conjugate of claim 25, an adjuvant and a physiologically acceptable carrier.
